SEQUENCE LISTING (5 sequences)

SEQ ID NO: 1 - DNA, 20 bp, Artificial sequence
  synthetic sequence
ggcagcctcactcgggttcc

SEQ ID NO: 2 - DNA, 20 bp, Artificial sequence
  synthetic sequence
aaggggtgattttgcccatt

SEQ ID NO: 3 - DNA, 20 bp, Artificial sequence
  synthetic sequence
aggaagatcaatggtatggt

SEQ ID NO: 4 - DNA, 20 bp, Artificial sequence
  synthetic sequence
ccaatcgagcgttctcgtgg

SEQ ID NO: 5 - DNA, 18 bp, Artificial sequence
  synthetic sequence
cagaccgaaccctaaaga

(19) United States
(12) Patent Application Publication
(10) Pub. No.: US 2024/0226126 A1
(43) Pub. Date: Jul. 11, 2024

(54) COMPOSITIONS FOR TREATMENT OF PSORIASIS

(71) Applicants: The Board of Trustees of the Leland Stanford Junior University, Stanford, CA (US); THE UNITED STATES GOVERNMENT AS REPRESENTED BY THE DEPARTMENT OF VETERANS AFFAIRS, Washington, DC (US)

(72) Inventors: M. Peter Marinkovich, Redwood City, CA (US); Atul Janardhan Butte, Menlo Park, CA (US); Mazen Nasrallah, Cambridge, MA (US); Marten CG Winge, Sunnyvale, CA (US)

(21) Appl. No.: 18/568,162

(22) PCT Filed: Jun. 15, 2022
(86) PCT No.: PCT/US2022/033670

§ 371 Date: Dec. 7, 2023

Related U.S. Application Data
(60) Provisional application No. 63/211,418, filed on Jun. 16, 2021.

(51) Int. Cl. A61K 31/7048; A61K 9/00; A61K 31/166; A61K 31/192; A61K 31/404; A61K 31/426; A61K 31/436; A61K 31/4439; A61K 31/48; A61K 31/519; A61K 31/5415; A61P 17/06

(52) U.S. Cl. CPC A61K 31/7048; A61K 9/0014; A61K 31/166; A61K 31/192; A61K 31/404; A61K 31/426; A61K 31/436; A61K 31/4439; A61K 31/48; A61K 31/519; A61K 31/5415; A61P 17/06

(57) ABSTRACT

Methods of treatment for psoriasis, and compositions for use in such methods are provided, utilizing repositioned drugs identified as anti-psoriasis agents.

### CROSS REFERENCE TO RELATED APPLICATION

The present application claims the benefit of and priority to U.S. Provisional Patent Application No. 63/211,418, filed Jun. 16, 2021, the entire disclosure of which is hereby incorporated by reference in its entirety.

### INCORPORATION BY REFERENCE OF SEQUENCE LISTING

A Sequence Listing is provided herewith as a Sequence Listing text, “STAN-1841WO_SEQUENCE_LISTING_ST25” created on Dec. 7, 2023 and having a size of 1,504 bytes. The contents of the Sequence Listing text are incorporated herein by reference in their entirety.

### BACKGROUND OF THE INVENTION

Psoriasis is a chronic immunoproliferative skin disease affecting approximately 2% of the world population. Despite a considerable understanding of psoriasis immunopathology, no cure currently exists. Current treatments are governed by the severity of presentation, response to conventional treatments, side-effect profiles and cost considerations.

Topical corticosteroids remain a therapeutic mainstay, however numerous side-effects, including skin atrophy, limit their use especially on face and intertriginous areas. Other topical therapies such as retinoids, tar derivatives and calcineurin inhibitors are used, but suffer from drawbacks of their own. Newer immune targeted approaches such as anti-IL-12, the IL-23p19, or anti-IL-17 therapies have proven efficacy. However cumulative costs and risks associated with systemic immunosuppressives, over the many years typically required for treatment of this chronic disorder, limit their use in many psoriasis patients. Thus more affordable therapies with fewer side effects are desirable.

Drug repositioning, the use of existing drugs for new therapeutic indications, is an exciting new field that offers great promise in providing cheaper and safer medications. The use of gene-expression profiles to establish ‘connections’ between drugs, diseases and genes is facilitated by the Connectivity Map, a publicly available compendium of 1309 drug gene-expression profiles, and the more recent Library of Integrated Network-Based Cellular Signatures (LINCS). The assimilation of Connectivity Map drug signatures and disease profiles from GEO and ArrayExpress has uncovered exciting and non-intuitive drug-disease connections, such as the efficacy of topiramate in IBD, and pyrvinium pamoate for liver cancer.

To address certain Connectivity Map limitations such as problems with batch effect and cell-line specific signatures, a method has been proposed of meta-analyzing drug gene-expression data using a rank aggregation algorithm which rank-aggregates drug signatures from individual CMap experiments, to derive a ‘consensus’ drug signature called the Prototype Ranked List (PRL). This enables construction of a drug-drug network that provides intuitive network visualization of drug connections, organizing drugs into communities enriched in common mechanism of action (MoA) and thus allows MoA-inferences.

Dermatologists and patients would benefit from new therapies for psoriasis, particularly those that can be delivered topically.

### SUMMARY OF THE INVENTION

Methods of treatment for psoriasis, and compositions for use in such methods are provided. In the methods of the invention, an effective dose of a repositioned drug, for example as set forth in Table 1, is administered to an individual suffering from psoriasis, where the dose is effective for reducing the Psoriasis Area and Severity Index of the individual. Agents of interest include, for example, Quinostatin; Bromocriptine; Trifluoperazine; Sirolimus; 0297417-0002B; 0198306-0000, PD-198306; PHA-00665752; PNU-0230031 [267429-39-0]; G-012559 [369370-06-9]; Etoposide; and Ethacrynic Acid. In certain specific embodiments the agent may additionally include benzamil. Such agents can also include derivatives and pro-drugs of compounds set forth in Table 1. Such agents may be referred to herein as an “anti-psoriasis agent”.

Repositioned drugs for the treatment of psoriasis are disclosed herein. These were identified by a process described herein. Multiple sets of gene expression data consisting of expression data from lesional and non-lesional skin were obtained and analyzed using the statistical software to hierarchically merge ranked lists to generate a single ‘prototype’ ranked list (PRL). The outcome was a consensus gene expression signature for lesional psoriatic skin compared to non-lesional skin. The psoriatic PRL was analyzed for mode of action using network analysis (MANTRA). A total enrichment-score (TES) distance was calculated using the MANTRA tool for each drug-PRL vs disease-PRL pair, which represents statistical measure of similarity between two gene signatures, ranging between 0 and 2, where the smaller TES values indicate increased similarity between the drug and disease signatures. A TES cutoff of 0.8 was chosen as a threshold of significance. At this level of significance, the repositioned drug candidates were identified as shown in Table 1. Validation of the approach is provided by the analysis of benzamil's effectiveness in treating psoriasis.

In some embodiments the anti-psoriasis agent is systemically administered, e.g. by i.p., i.m., i.v. injection, etc. In other embodiments the anti-psoriasis agent is topically administered, e.g. formulated as a patch, lotion, gel, microneedle array, intralesional injection, etc. In some embodiments, formulations for topical administration are provided. In some embodiments, the anti-psoriasis drug may be the only active agent in the formulation. The anti-psoriasis agent can be formulated in combination with other agents effective in the treatment of psoriasis, e.g. corticosteroids, vitamin D analogs, retinoids, and the like. The anti-psoriasis agent can be administered in combination with systemic agents as well, e.g. immunosuppressants, anti-cytokine therapies, T-cell-targeted therapies, and the like.

In some embodiments, methods for treating psoriasis, e.g., chronic psoriasis, using a repositioned drug as described herein. The flare of psoriasis may be indicated by loss of a Psoriasis Area and Severity Index (PASI) 90 response, by loss of a Psoriasis Area and Severity Index (PASI) 75 response, by loss of a Psoriasis Area and Severity Index (PASI) 50 response, or by loss of a clear or minimal Physician's Global Assessment (PGA) rating. The loss of a PASI response may be loss of PASI response of a single body region, loss of PASI response of two body regions, loss of PASI response of three body regions, or loss of PASI response of four body regions. The body region may be trunk, lower extremities, upper extremities, or head and neck.

In one embodiment, the psoriasis is chronic psoriasis. In one embodiment, the psoriasis is plaque psoriasis, e.g., chronic plaque psoriasis. In another embodiment, the psoriasis is chronic psoriasis, e.g., chronic plaque psoriasis. In yet another embodiment, the psoriasis is moderate to severe psoriasis, e.g., moderate to severe plaque psoriasis, moderate to severe chronic psoriasis or moderate to severe chronic plaque psoriasis. In one embodiment, the subject has had a clinical diagnosis of psoriasis for at least 6 months. In another embodiment, the subject has had stable plaque psoriasis for at least 2 months.

### BRIEF DESCRIPTION OF THE DRAWINGS

FIG. 1. Drug repositioning and target identification in psoriasis. (A) Drug repositioning flowchart. (B) GEA of psoriatic lesional skin (LS) vs non-lesional skin (NLS) using gene ontology gene sets (FDR≤0.001), with overlap of genes from the benzamil gene-set (p-value≤0.10, hypergeometric test). Nodes represent gene sets. Size of nodes correlate with the size of the gene-sets. Edges represent connections between gene-sets with an overlap coefficient of 0.60 or less, with the size of each edge corresponding to the strength of overlap. Red nodes are positively enriched in psoriatic lesional skin compared to non-lesional skin. Blue nodes are negatively enriched in psoriatic lesional skin compared to the non-lesional skin. (C) Heatmap of normalized GEO mRNA expression from control (C), non-lesional (NL) or lesional (L) psoriatic skin for benzamil targets SCNN1α, NCX1, TRPV3, ASIC1 and NHE. (D) Heatmap of mRNA expression by RT-qPCR of benzamil targets assayed in keratinocytes, fibroblasts, melanocytes, CD3+ and CD3− PBMCs. (C-D) n=1-3 per condition. Normalized to internal 18S, then fold change relative keratinocyte SCNN1α.

FIG. 2. Benzamil attenuates psoriasiform hyperplasia in vivo. (A) Rac1_{V12} transgenic mice were injected intraperitoneally every other day for 20 injections with benzamil (2 mg/kg) or saline control. (B) Treated mice exhibited marked signs of improvement, including reduced scaling, erythema and edema on muzzle and ears and healing of tail lesions. (C) Benzamil-treated skin exhibited reduced epidermal hyperproliferation in a dose-dependent. (D-E) To control benzamil release, Rac1_{V12} mice were also implanted osmotic pumps containing benzamil or vehicle control for 2×14 days, (F) significantly reducing epidermal thickness despite a 30% shorter treatment duration than IP injected mice. (I-J) Tail lysates of mice treated with benzamil or vehicle were immunoblotted and analyzed for IKBα (K-L) and phospho-STAT3 expression. (G) Immunofluorescence staining of ki67 (red) in benzamil-treated mice compared to vehicle control in basal and subrabasal (relative the basement membrane, integrin A6, green) epidermal layers. (H) Quantification of ki67 expression demonstrated a significant reduction in both basal and suprabasal layers in treated skin. (M) Immunofluorescence staining of CD3+ cells (red) in treated compared to vehicle skin (N) demonstrated a significant reduction in skin infiltrating CD3+ cells. (A) 2.5× Scalebar 200 μm, inset 10× scalebar 50 μm. Linear regression with upper and lower confidence limit (E) 10× tiled whole-section scalebar 500 μm. (K) 10× scalebar 100 μm. (N) 20×, scalebar 100 μm. (A) n=4. (B-C) n=3 per condition. (D) n=3-10 per condition. (E-F) n=3-9 per condition. (G-J) n=3 per condition. (K-N) n=3-9 per condition. (D) *=p<0.05, **=p<0.005 unpaired two-tailed test with Welch correction. (G) *=p,0.05 unpaired one-tailed t-test with Welch correction (F, L, N) **=p<0.005 unpaired two-tailed t-test with Welch correction. (C,E) Arrowhead denotes representative areas of epidermal acanthosis.

FIG. 3. Topical delivery attenuates psoriasis in both murine and human reconstructed psoriasis skin. (A) (B) Topically delivered benzamil) reduced epidermal hyper proliferation to a similar extent as potent topical corticosteroids (clobetasol) compared to vehicle. (C) Quantification of epidermal thickness between topical vehicle, benzamil and clobetasol. (D) Immunofluorescence staining of Ki67 (red) relative basement membrane (ITA6, green). (E) Ki67 quantification demonstrated a significant reduction in both basal and suprabasal layers between benzamil and vehicle. (F) Schematic of workflow for generation of xenografts from primary human patient or control cells. Xenografts were treated with TB or vehicle control for 15 days after bandage removal and intradermal injection of autologous PBMCs. (G) Vehicle treated patient grafts showed pronounced psoriasiform hyperplasia, which was abolished following topical benzamil. (H) Immunofluorescence staining analysis showed diminished epidermal ki67, (I) CD3 infiltration and (J) phospho-stat3 expression. Quantification of (K) epidermal thickness, (L) Ki67+ epidermal cells, (M) PhosphoSTAT3+ epidermal cells and (N) epidermal and dermal CD3+ T-cells. (A) n=3-5 biological replicates per condition. Unpaired two-tailed t-test. CCL7 one-tailed unpaired t-test. (B-E) n=4-8 biological replicates per condition. Mann-Whitney two-tailed test. (H-N) n=2-3 biological replicates per condition. (B) 2.5× magnification, inset 10× magnification (D, G) 10× magnification scalebar 100 μm, (H-J) 20× magnification scalebar 50 μm. (D,H-J) Ki67, PSTAT3, CD3-red, ITGA6-green, DNA blue. (B, G) Arrowhead denotes representative areas of epidermal acanthosis.

FIG. 4. Experimental validation of benzamil efficacy in reconstructed organotypic 3D psoriasis skin equivalents through Rac1-inhibition in keratinocytes. (A) The half maximal inhibitory concentrate (IC50) of benzamil on proliferation in cells from 5 psoriasis donors (primary psoriasis keratinocytes) was determined to be approximately 1 μM. (B) Using a concentration of 10 μM, there was a significant decrease in paired PSOKC monolayer cultures compared to PCKC cultures. (C) Comparing primary keratinocytes transduced with V12 or LacZ control, the IC50 of V12KC were 0.99 μM, indicating a selective Rac1-inhibitory effect. (D) Pulling down Rac1-GTP using PAK1-PDB in unstimulated or EGF stimulated PSOKC or PCKC, (E) Benzamil significantly reduced prolonged Rac1-activity, distinct from control cells. (F) Benzamil and Zoniporide, but not Amiloride or Triamterene significantly reduced intracellular pH in PSOKCs. (G) Similarly, Zoniporide but not amiloride or triamterene reduced proliferation in PSOKCs to levels observed by Benzamil. (H) Confocal microscopy of Immunofluorescence-stained organotypic acellular dermal support (ADS) 3D skin equivalents comprising of control (upper) or psoriasis (lower) keratinocytes and fibroblasts seeded onto devitalized dermis and grown at the airfluid interphase for 7 days in KGM media without (left panels) or supplemented with IL17, TNFα and IL22 (middle and right panels) and with cytokine supplementation and benzamil (right panels). Ki67-red, ITGA6-green DNA-blue. I) Benzamil significantly reduced hyperproliferatory cells (Ki67) to unstimulated levels in psoriasis ADS. (J) Stimulation with IL17, TNFα and IL22 lead to significant epidermal hyperplasia in psoriasis ADS, and was normalized by benzamil. (C/P-control/psoriasis ADS, CS/PS-control/psoriasis stimulated ADS, CSB/PSB-control/psoriasis stimulated ADS benzamil. (H) (A) Scalebar 50 μm (inset 50 μm). (A) n=5 biological replicates and 3-8 technical replicates per condition. (B-E,H) n=3-4 biological replicates per condition. (H-I) n=3 biological replicates per condition. ((B-C) *=p<0.05 unpaired 2-tailed t-test with Welsh's correction. (E, G, I) Unpaired two-tailed t-test with Welsh's correction. (J) n=3 biological replicates in 4 technical replicates per condition. **=p<0.005 Paired t-test. (K-L) n=4 biological replicates per condition. *=p<0.05 837 Ratio paired two-tailed t-test.

FIG. 5. Aberrant Rac1-NCK1 interactions are reduced by benzamil. (A) The E. coli biotin ligase BirA was fused to Rac1 and biotinylated proteins within a ˜10 nm radius of the bait was identified using through a proximity-dependent biotinylation technique, and known Rac1-interactors including COL17A1, ITGB1, EGFR and CD44 identified. (B) Using stringdb the Rac1-interactome was mapped and enriched using KEGG including MAPK signaling, actin cytoskeleton, adherence junction, focal adhesion and bacterial epithelial invasion. (C) Enriched proteins from BIOID analysis through BirA tagging the N terminal end of Rac1, comparing benzamil-mediated alterations in protein-protein interactions in psoriatic primary keratinocytes. Enriched interactors with Rac1 in psoriasis patient keratinocytes were annotated using STRING (inset), and NCK1 (red) predicted to directly interact with Rac1. (D) Rac1-NCK1 was not significantly enriched in primary control keratinocytes (D). (E) Validation of BiolD by co-Immunoprecipitation of NCK1 and Rac1 in primary psoriatic or control keratinocyte (F). CRISPR/Cas9 knockdown of NCK1 in primary keratinocytes. (G) Rac1GTP pulldown of Crispr/Cas9 (cr) scramble or crNCK1 knockout psoriasis and control keratinocytes showing reduced prolonged Rac1GTP in psoriasis patient cells. (H-I) crNCK1 mimicked benzamil mediated reduction in p-p44/42. (A-D) n=2 biological replicates per condition (E) n=3 paired biological replicates per condition (psoriasis and control). (F) n=2 biological and 2 technical replicates per condition. (G) n=2 biological and 2 technical replicates per condition (H-I) n=2 biological and 2 technical replicates per condition (I) *=p<0.05 two-tailed paired t-test. (F) Enriched proteins with saint-score above 0.9 marked in gray.

FIG. 6. Mechanism for benzamil-mediated inhibition of pathologic Rac1 activation in psoriatic cells. (A) Proximity ligation assay (PLA) of Rac1-NCK1 in psoriatic and control, keratinocytes, treated with vehicle or benzamil (10 uM). Puncta demarcating proximity in red. (B) Quantification of puncta in conditions outlined in (A). (C) Proximity ligation assay (PLA) of psoriatic and control skin demonstrated marked proximity of NCK1 and Rac1 (positive proximity in red) in psoriatic lesional skin compared to control skin. (D) Quantification of PLA signal showed a significant increase in psoriatic skin compared to control skin. (E) PLA of Rac1-NCK1 in psoriasis and control xenografts treated with topical benzamil or vehicle. (F) Quantification of Rac1-NCK1 PLA signal intensity in vehicle or drug treated xenografts. (G) Schematic of mechanism of action for benzamil. (1) Benzamil inhibits NHC1 and SCNN1. (2). Inhibition of NHE1 and SCNN1 inhibits NCK1 binding to Rac1. (3). Rac1 inhibition leads to reduced ‘psoriatic related gene transcription.’ (4) Keratinocyte proliferation is reduced. (5) Production of chemokines are slowed. (6) Less chemokines results in less chemotaxic immune cells. (7) Immunocytes produce less proinflammatory cytokines. (8) Reduced proinflammatory signals slow down keratinocyte proliferative response. (A-B) n=3-7 replicates per condition of 4-12 cells each per 63× field of view per coverslip. (C-D) n=3-5 biological replicates per condition (E-F) n=1-2 biological replicates per condition with n=2 technical replicates each. (A) 63× magnification, scalebar 20 μm. (C) 20× magnification scalebar 50 μm, inset 63× magnification, scalebar 20 μm. (E). 20× magnification scalebar 50 μm, inset 63× magnification scalebar 20 μm. PLA positive punctae red, DNA blue. (B) **=p<0.005 Mann-Whitney unpaired two-tailed test. (D) *=p<0.05 Mann-Whitney unpaired two-tailed test. (F) *=p<0.05 ratio two-tailed paired t-test.

FIG. 7. Bromocriptine activity in treating psoriasis.

FIG. 8. Transcriptional signature of benzamil expression and targets in psoriatic and control skin and primary cells. (A) Psoriasis microarray data was downloaded from NCBI Gene Expression Omnibus (GEO) for Benzamil targets. (B) mRNA expression by rt-QPCR of benzamil targets in keratinocytes, fibroblasts, melanocytes, and CD3+ and CD3− immunocytes. (C) Targeted CRISPR-Cas9 screen of benzamil targets SCNN1α (light grey), NCX1 (dark grey), NHE (black, y-axis) and NHE and SCNN1α merged (blue) compared to benzamil (red, x axis) relative benzamil signature genes. (D) mRNA expression of 10 of the top ranked transcripts from the drug repurposing algorithm was compared using RT-qPCR between SCNN1α NCX1 and NHE knockout keratinocytes compared to Benzamil treated keratinocytes. (E) Most significant Biocarta ontology terms for the paired psoriasis-benzamil signature included T-cell activation, MAPK signaling, keratinocyte differentiation and Rac1 signaling. GTEX expression demonstrating expression in SCNN1A (F), NHE1 (G) and combined SCNN1A and NHE1 (H). (A) n=58-64 per condition. (B) n=1-3 per condition. (C) n=3 per condition (D) n=1-3 per condition. (E) n=58 per 1091 condition. (C) Linear regression analysis, *=p<0.05.

FIG. 9. Targeted CRISPR-cas9 and siRNA screen of benzamil targets in primary cells. (A) mRNA expression in CRISPR-cas9 knockout of SCNN1α, NCX1 and NHE respectively compared to scramble control, by RT-qPCR. (B) siRNA knockdown of SCNN1α, and NCX1 compared to siRNA scramble control, by RT-qPCR. (C) Efficacy of protein knockout by immunofluorescence using confocal microscopy for NHE (top), SCNN1α (middle) and NCX1 (bottom). (D) Linear regression analysis of siSCNN1α or NCX1 compared benzamil signature genes also yielded a non-significant deviation from baseline, similar to respective CRISPR-cas9 conditions. (C) 20× magnification scalebar 50 μm. NHE/SCNN1α/NCX1—red, DNA blue.

FIG. 10. Effect of benzamil inhibits Rac1 in vivo, and alters expression of psoriasis-associated signaling pathways. (A) Tail sections of Rac1_{V12} mice treated with vehicle or benzamil (IP, 2 mg/kg/day) or vehicle control were assayed by immunofluorescence using confocal microscopy for antibodies against (A) Ki67, (B) TGFa, (C) phospho-STAT3, (D) phospho-RelA and (E) CD3. (F) Benzamil significantly reduced mRNA expression of (F) tnfa and (G) krt16, whereas (H) il17 was not significantly reduced by RT-qPCR (A, E) 20× magnification scalebar 100 μm, (B-D) 63× magnification, scalebar 50 μm. Ki67, TGF^{α}, PSTAT3, PrelA, CD3-red, DNA blue.

FIG. 11. Efficacy of benzamil delivered through osmotic pumps. (A) Representative H&E section from four mice treated with benzamil (1-4) or (B) vehicle control (5-8) using osmotic pumps. (C) Luminex from skin lysates of systemically treated mice showed a reduction in chemotactic cytokines CXCL1, CCL2, CXCL2, CCL7 as well as TNFα. (A-B) n=7-9 biological replicates per condition.

FIG. 12. Wholemount images of benzamil or vehicle treated Rac1_{V12} mice. Whole mounts of serial 10× images of (A) Rac1_{V12} mice treated with benzamil using osmotic pumps (B) Rac1_{V12} vehicle control or (C) WT vehicle control. (A-B) n=7-9 biological replicates per condition (C) n=3.

FIG. 13. Effect of benzamil on cytokine expression by primary psoriasis keratinocytes. (A) Heatmap of significantly altered cytokines in primary cultures of PSOKC by luminex assays in media, media with vehicle or media with 10 μM of benzamil. (B) Fold change of benzamil effect in PSOKCs. (C) KEGG pathway analysis of significantly altered cytokines by benzamil in primary psoriasis keratinocyte cultures (A-B) Two-tailed unpaired t-test p-value <0.05. (C) Odds ratio left Y axis −log(P) right Y axis. (A-C) n=3 biological replicates per condition.

FIG. 14. Effect of benzamil on primary psoriatic keratinocyte proliferation and Rac1 activity. (A) IC50 in five psoriasis donor primary keratinocyte cultures showed an IC50 range from 0.0001-4.6 μM. (B-E) 10 μM (B) Cytokine stimulation with TNFa, IL17 and IL22 for 24 hours significantly reduced proliferation in psoriatic keratinocytes, which was not recapitulated in non-cytokine stimulated cells (stimulated with EGF), to the extent of 48 hours of non-stimulated cells. (C) Benzamil significantly reduced V12 Rac1 GTP activity in V12KC (D). Benzamil markedly reduced nuclear translocation of phosphorylated STAT, assayed in primary psoriatic 2D cultures, as well as in organotypic 3D skin equivalents of primary psoriasis or control keratincoytes (E). (D-E) pSTAT3 (red) Rac1GTP (green) DNA (blue). (D) 63×, (E) 20×.

FIG. 15. Th17 differentiation assay and assessment of benzamil effect. (A) Schematic for Th17 differentiation and incubation with benzamil. (B-H) Gating strategy and FMO control gating (right panel E-H). (D) To explore an effect of benzamil on IL17 and IL22 producing cells, naïve human CD4 T cells were isolated from PBMCs and differentiated into Th17 immunocytes, in the presence or absence of benzamil. No significant difference in (D-E) ROR^{γ}t, (F-G) IL17 or (H-I) Rac1_{GTP} was observed in benzamil treated immunocytes. V-vehicle, B-benzamil.

FIG. 16. Benzamil inhibits aberrant Rac1-NCK1 interactions in organotypic skin equivalents using primary psoriasis patient cells. (A) Workflow for generating organotypic 3D skin equivalents using primary patient keratinocytes and fibroblasts seeded onto acellular dermal support (ADS). (B) Proximity ligation assays of Rac1-NCK1 in control ADS cultured in KGM media or (C) psoriasis cells in KGM media, or (D) supplemented with IL17, IL22, TNFα and (E) cytokines and benzamil 10 uM for 7 days. (F) Patient ADS exhibited significantly increased Rac1-NCK1 punctae compared to unstimulated, which was significantly reduced in ADS treated with benzamil (B-E) 63× magnification, bar=50 μM. n=3-4 biological replicates per condition.

FIG. 17. Benzamil modulates Rac1 activity in keratinocytes and correlates with altered intracellular pH. (A) Benzamil, but not amiloride significantly reduced intracellular pH in psoriatic keratinocytes.

FIG. 18. Effect of benzamil on MAPK signaling in primary psoriasis keratinocytes. (A) Pooled heatmap of top altered phospho/total residues from four MAPK phosphor-array of lysates from primary psoriasis patient or control keratinocytes incubated with vehicle or benzamil. Representative immunoblots of validation of (B) p-p44/42 and (C) p-SAPK/JNK ratios from patient and control keratinocytes incubated in the presence of benzamil or vehicle. (D-E) Quantification of densitometry showing significant reduction of both p-p44/42 and SAPK/JNK in psoriatic keratinocytes by benzamil. (F) CRISPR/Cas9 knockout of NCK1 (crNCK1) yielded a (non-significant) trend of downregulation of phosphor-IRS in psoriasis patient, but not control keratinocytes, quantified in (G). (H) crNCK1 significantly reduced cRAF in psoriasis patient but not control keratinocytes, quantified in (I). (J-K) crNCK1 mimicked benzamil mediated reduction in phospho-SAPK/JNK although not the phosphor-total SAPK/JNK ratio. (L) To additionally assess Rac1GTP dependency for p-SAPK/JNK in psoriasis keratinocytes, psoriasis keratinocytes were also transduced with a dominant negative mutant of Rac1 (Rac1N17), which rendered a reduced and benzamil-resistant expression or p-SAPK/JNK. (A) n=1 per condition (B-E) n=4-5 paired biological replicates per condition (psoriasis and control). (F) n=2 biological replicates per condition. (H) n=2 technical replicates per condition (1) n=2 per condition (J) n=2 technical replicates per condition (K-N) n=2 paired biological replicates per condition with 6 technical replicates per spot. (L) n=2 technical replicates per condition. (D, E, L, N) *=p<0.05 two-tailed paired t-test.

FIG. 19. A. Dose dependent decrease of CXCL1 by bromocriptine (from 1 nM), Trifluoperazine (from 1 nM), Quinostatin (from 1 nM), Etoposide (from 1 nM), Etacrynic acid (at 1, 5 and 10 uM), PHA-665752 (at 5 and 10 uM) and PD198306 (at 100 nM). B. A decrease of IL-8 noted by Bromocriptine (at 1, 5 and 10 uM), Trifluoperazine (at 5 and 10 uM), Quinostatin (at 1, 5 and 10 uM), etoposide (at 10, 100 nM and 1, 5 and 10 uM), Etachrynic acid (at 10 uM). C. Decrease of IL6 by Bromocriptine (from 1 nM), Trifluoperazine, Quinostatin, Etoposide, Ethacrynic acid, PHA-665752 and PD198306 (all from 1 nM)

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Repositioned drugs for use in the treatment of psoriasis are listed below in Table 1.

TABLE 1

| | Total | | |
|---|---|---|---|
| | Enrichment | | |
| Compound | Score | Class | Mechanistic Logic |
| 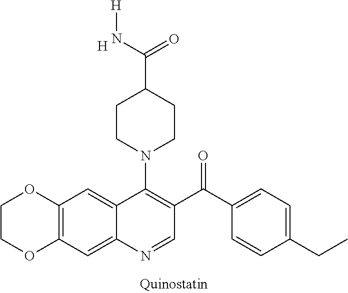 | 0.68 | Class Ia PI3K Inhibitor 1 | PIK3-AKT-mTOR pathway implicated in psoriasis |
| 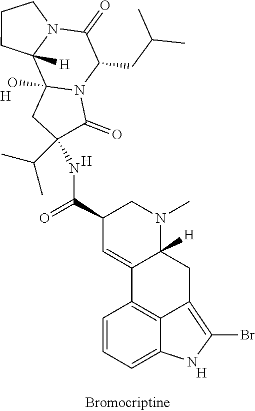 | 0.75 | Dopamine Agonist | Inhibits epidermal hyperplasia; T-cell immunomodulator |
| 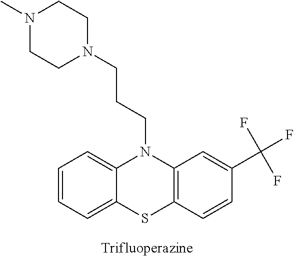 [SMILES: CN1CCN(CCCN2c3ccccc3Sc3ccc(C(F)(F)F)cc32)CC1] | 0.75 | Dopaminergic anatagonist, Calmodulin Antagonist | Inhibits keratinocyte proliferation; decreases neutrophil degranulation; inhibits T-cell activation; inhibits TLR activation |
| 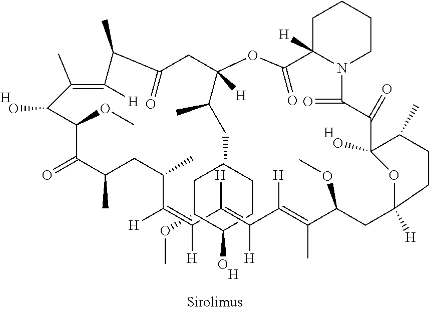 | 0.77 | mTOR antagonist | PI3K-AKT-mTOR pathway implicated in psoriasis 2 |
| 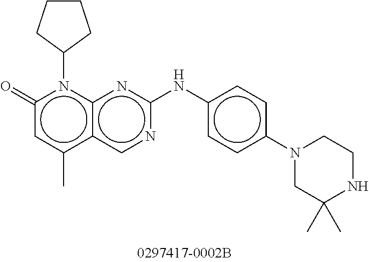 | 0.77 | macrophage colony stimulating factor 1 receptor | |
| 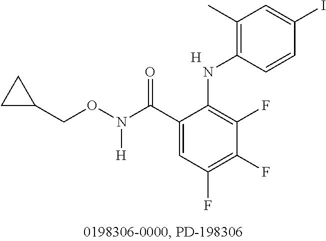 | 0.78 | MP2K1 | |
| 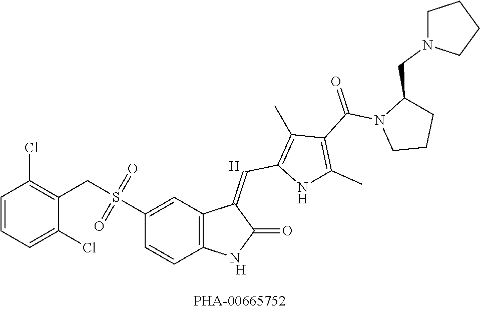 | 0.78 | Tyrosine-protein kinase; HIPK3; BMP2K | |
| 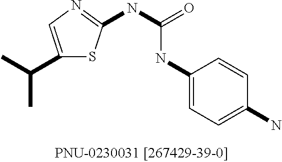 | 0.79 | Tyrosine protein kinases, c-SRC, Yes, Fyn | |
| 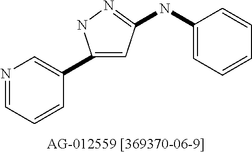 | 0.79 | Tyrosine protein kinases, c-SRC, Yes | |
| 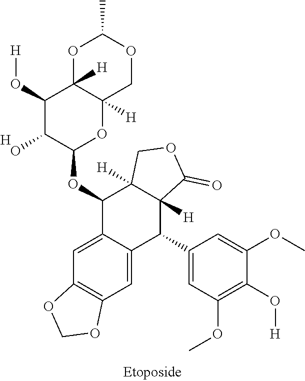 | 0.79 | Topoisomerase Inhibitor | Anti-proliferative effects of topoisomerase inhibitors |
| 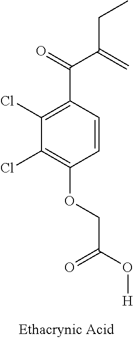 [SMILES: C=C(CC)C(=O)c1ccc(OCC(=O)O)c(Cl)c1Cl] | 0.79 | Diuretic | |

Salts, solvates, hydrates, and prodrug forms of a compound set forth in Table 1 are of interest as therapeutic agents. Polymorphic, pseudo-polymorphic, amorphous and co-crystal forms of a compound are also of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

The compounds of the invention may be administered as described herein in Table 1, or in a form from which the active agent can be derived, such as a prodrug. A “prodrug” is a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of the compound. Additional types of prodrugs are also encompassed. For instance, free carboxyl groups can be derivatized as amides or alkyl esters. Prodrug esters as employed herein includes esters and carbonates formed by reacting one or more hydroxyls of compounds of the method of the invention with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula I) is a prodrug within the scope of the invention. Various forms of prodrugs are well known in the art. A comprehensive description of prodrugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991).

The term “Alkyl” refers to a C₁-C₂₀ alkyl that may be linear, branched, or cyclic. “Lower alkyl”, as in “lower alkyl”, or “substituted lower alkyl”, means a C₁-C₁₀ alkyl. The term “alkyl”, “lower alkyl” or “cycloalkyl” includes methyl, ethyl, isopropyl, propyl, butyl, isobutyl, sec-butyl, tert-butyl, pentyl, neopentyl, hexyl, cyclopropyl, cyclopropylmethyl, cyclobutyl, cyclobutylmethyl, cyclopentyl, cyclopentylmethyl, cyclohexyl, cyclohexylmethyl, C₆ to C₁₂ spirocycles, cyclopropylethyl, cyclobutylethyl, decalinyl, Bicyclo-[1.1.1]-pentyl, norboranyl, bicylo-[2.2.2]-octyl, cubyl, adamantanyl and related cage hydrocarbon moieties. In certain embodiments, the alkyl is a C₁-C₂₀ alkyl. In certain embodiments the alkyl group is poly deuterated.

A “substituted alkyl” is an alkyl which is typically mono-, di-, or tri-substituted with heterocycloalkyl, aryl, substituted aryl, heteroaryl, nitro, cyano (also referred to herein as nitrile), azido, halo, —OR, —SR, —SF₅, —CHO, —COR, —C(O)OR, —C(O)—NR₂, —OC(O)R, —OC(O)NR₂, —OC(O)OR, —P(O)(OR)₂, —OP(O)(OR)₂, —NR₂, —N+R₃ (wherein a counterion may be present), —CONR₂, —NRCOR, —NHC(O)OR, —NHC(O)NR₂, —NHC(NH)NR₂, SO₃, —SO₂OR, —OSO₂R, —SO₂NR₂, or —NRSO₂R, where each R is, independently, hydrogen, lower alkyl, R′-substituted lower alkyl, aryl, R′-substituted aryl, heteroaryl, heteroaryl(alkyl), R′-substituted aryl(alkyl), or aryl(alkyl) and each R′ is, independently, hydroxy, halo, alkyloxy, cyano, thio, SF₅, nitro, alkyl, halo-alkyl, or amino. Substituted alkyls which are substituted with one to three of the substituents selected from the group consisting of alkynyl, cyano, halo, alkyloxy, thio, nitro, amino, or hydroxy are particularly of interest.

The term “Aryl” refers to an aromatic ring having (4n+2) pi electrons that may contain 6 to 20 ring carbon atoms, and be composed of a single ring (e.g., phenyl), or two or more condensed rings, such as 2 to 3 condensed rings (e.g., naphthyl), or two or more aromatic rings, such as 2 to 3 aromatic rings, which are linked by a single bond (e.g., biphenylyl). In certain cases, the aryl is C₆-C₁₆ or C₆ to C₁₄. In certain embodiments the alkyl group has one or more hydrogen atoms replaced with deuterium.

Heteroaryl means an aromatic ring system containing (4n+2)pi electrons and comprised of 1 to 10 ring carbon atoms and 1 to 5 heteroatoms selected from O, N, S, Se, having a single ring (e.g., thiophene, pyridine, pyrazine, imidazole, oxazole, tetrazole, etc.), or two or more condensed rings, for example 2 to 3 condensed rings (e.g., indole, benzimidazole, quinolone, quinoxaline, phenothiazine, etc.), or two or more aromatic rings, such as 2 to 3 aromatic rings, which are linked by a single bond (e.g., bipyridyl). In some cases, the heteroaryl is C₁-C₁₆, and a selection of 1 to 5 heteroatoms consisting of S, Se, N, and O.

The term “heterocycloalkyl”, “heterocycle”, “heterocyclic group” or “heterocyclyl” refers to a saturated or unsaturated nonaromatic ring system containing 1 to 10 ring carbon atoms and 1 to 5 heteroatoms selected from O, N, S, Se, having a single ring (e.g., tetrahydrofuran, aziridine, azetidine, pyrrolidine, piperidine, tetrathiopyran, hexamethylene oxide, oxazepane, etc.), or two or more condensed rings, such as 2 to 3 condensed rings (e.g., indoline, tetrahydrobenzodiazapines, etc., including fused, bridged and spiro ring systems, having 3-15 ring atoms, included 1 to 4 heteroatoms. In certain cases, the heterocycloalky is C₁-C₁₆, and a selection of 1 to 5 heteroatoms consisting of S, Se, N, and O. In fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, benzimidazole, pyrazole, benzopyrazole, tetrazole, 1,2,3-triazole, benzotriazole, 1,2,4-triazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, benzoisothiazole, phenazine, isoxazole, benzoisooxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, benzothiazole, thiazolidine, furan, benzofuran, thiophene, benzothiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzotetrahydrofuranyl, and the like.

Substituted heterocycloalkyl, aryl, heteroaryl are optionally substituted with, hydrogen, 1 to 3 alkyl, alkenyl, alkynyl, cycloalkyl, cycloalkyl(alkyl), aryl, substituted aryl, aryl(alkyl), —SO₂NR⁵R⁵, —PO₃H₂, —NR⁵SO₂R⁶ or —NR⁵C(═O)R⁶, wherein R⁵ and R⁶ are independently, hydrogen, alkyl, alkenyl, alkynyl, cycloalkyl, cycloalkyl(alkyl), aryl, optionally substituted heterocycloalkyl, aryloxy, heteroaryl, heteroaryl(alkyl), or R⁵ and R⁶ together are —(CH₂)₃₋₆— or —(CH₂)₀₋₃X(CH₂)₀₋₃— where X═NR, O, S, SO₂, substituted aryl(alkyl), halo(alkyl), SF₅, NR⁵₃_{⁺}, azido, cyano (also referred to herein as nitrile), —OR⁵, —SR⁵, —NR⁵R⁶, halogen, nitro, SCH₃, OCF₃, SO₂CH₃, SCF₃, SO₂CF₃, CF₃, —SO₂OR⁵, —OSO₂R⁵, CCl₃, —C(═O)R⁵, —C(═O)OR⁵; —C(═O)NR⁵R⁶, —OC(═O)R⁵.

By “substituted” as in “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-alkynyl, SO₂-substituted alkynyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—. Sulfonimidoyl refers to S(O)(NH)-bonded as for sulfonyl defined above.

The term “water-soluble group” refers to a functional group that is well solvated in aqueous environments and that imparts improved water solubility to the compound to which it is attached. Water-soluble groups of interest include, but are not limited to, polyalcohols, straight chain or cyclic saccharides, primary, secondary, tertiary, or quaternary amines and polyamines, sulfate groups, sulfonate groups, sulfinate groups, carboxylate groups, phosphate groups, phosphonate groups, phosphinate groups, ascorbate groups, glycols, including polyethylene glycols (PEG) and modified PEGs, and polyethers. In some instances, water-soluble groups are primary, secondary, tertiary, and quaternary amines, carboxylates, phosphonates, phosphates, sulfonates, sulfates, —N(H)₀₋₁(CH₂CH₂OH)₁₋₂, —NHCH₂CH₂N(CH₃)₂₋₃, —NHCH₂CH₂SO₃H, —NHCH₂CH₂PO₃H₂ and —NHCH₂CH₂CO₂H, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, oligoethylene glycol, and polyethylene glycol, wherein yy is selected from 1 to 1000, X is selected from 0, S, and NR^{ZZ}, and R^{ZZ} and R^{YY} are independently selected from H and C1-3 alkyl.

The term “carboxy isostere” refers to standard medicinal bioisosteric replacement groups for carboxylic acids, amides and ester. These include, but are not limited to: acyl cyanamide, tetrazoles, hydroxychromes, 3-hydroxy-1,2,4-triazoles, 1-hydroxy pyrazoles, 2,4-dihydroxy imidazoles, 1-hydroxy imidazole, 1-hydroxy 1,2,3-triazole, alkylsulfonyl carboxamides, hydroxy isoxazoles, 5-hydroxy 1,2,4-oxadiazoles, thiazoles, 1,2,4-oxadiazoles, 1,2,4-oxadiazolones, oxazoles, triazoles, thiazoles, others hydroxamic acids, sulfonimide, acylsulfonamide, sulfonylureas, oxadiazolone, thiazolidinediones, oxadiazole, thiadiazole, isothiazoles, difluorophenols, tetramic acids, tetronic acids, squaric acids, hydroxyquinoline-ones, hydroxyquinoline-2-ones, boronic acids and phosphoric acids.

As used herein the term “PEG” refers to a polyethylene glycol or a modified polyethylene glycol. Modified polyethylene glycol polymers include a methoxypolyethylene glycol, and polymers that are unsubstituted or substituted at one end with an alkyl, a substituted alkyl or a substituent (e.g., as described herein).

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+=C—), cyanato (—O—C≡N), isocyanato (—O—N+≡C—), isothiocyanato (—S—C≡N), azido (—N═N+=N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S— alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂OM⁺, —SO₂OR⁷⁰, —OSO₂R⁷⁰, —OSO₂OM⁺, —OSO₂OR⁷⁰, —P(O)(O)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl, N-morpholinyl, —N(H)₀₋₁(CH₂CH₂OH)₁₋₂, —NHCH₂CH₂N(CH₃)₂₋₃, —NHCH₂CH₂SO₃H, —NHCH₂CH₂PO₃H₂ and —NHCH₂CH₂CO₂H.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Salts include but are not limited to: Na, K, Ca, Mg, ammonium, tetraalkyl ammonium, aryl and alkyl sulfonates, phosphates, carboxylates, sulfates, C, Br, and guanidinium.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “heterocycloalkyl(alkyl)” refers to the group (heterocycloalkyl)-(alkyl)-.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Chronic Plaque Psoriasis. Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis.

Guttate Psoriasis. Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

Inverse Psoriasis. Inverse psoriasis is a form of psoriasis in which the patient has smooth, usually moist areas of skin that are red and inflamed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irritate the affected areas.

Pustular Psoriasis. Pustular psoriasis, also referred to as palmar plantar psoriasis, is a form of psoriasis that causes pus-filled blisters that vary in size and location, but often occur on the hands and feet. The blisters may be localized, or spread over large areas of the body. Pustular psoriasis can be both tender and painful, can cause fevers.

Erythrodermic Psoriasis. Erythrodermic psoriasis is a particularly inflammatory form of psoriasis that often affects most of the body surface. It may occur in association with von Zumbusch pustular psoriasis. It is a rare type of psoriasis, occurring once or more during the lifetime of 3 percent of people who have psoriasis. It generally appears on people who have unstable plaque psoriasis. Widespread, fiery redness and exfoliation of the skin characterize this form. Severe itching and pain often accompany it. Erythrodermic psoriasis causes protein and fluid loss that can lead to severe illness. Edema (swelling from fluid retention), especially around the ankles, may develop, along with infection. Erythrodermic psoriasis also can bring on pneumonia and congestive heart failure. People with severe cases often require hospitalization. Erythrodermic psoriasis can occur abruptly at the first signs of psoriasis or it can come on gradually in people with plaque psoriasis. Combination treatments are frequently required, for example topical products and one or two systemic medications.

The term “sensitivity” and “sensitive” when made in reference to treatment is a relative term which refers to the degree of effectiveness of a treatment compound in lessening or decreasing the symptoms of the disease being treated. For example, the term “increased sensitivity” when used in reference to treatment of a cell or patient refers to an increase of, at least a 5%, or more, in the effectiveness in lessening or decreasing the symptoms of psoriasis when measured using any methods well-accepted in the art.

As used herein, and unless otherwise specified, the term “therapeutically effective amount” of a compound is an amount sufficient to provide a therapeutic benefit in the treatment or management of psoriasis, or to delay or minimize one or more symptoms associated with psoriasis. A therapeutically effective amount of a compound means an amount of therapeutic agent, alone or in combination with other therapies, which provides a therapeutic benefit in the treatment or management of psoriasis. The term “therapeutically effective amount” can encompass an amount that improves overall therapy, reduces or avoids symptoms or causes of psoriasis, or enhances the therapeutic efficacy of another therapeutic agent.

The term “likelihood” generally refers to an increase in the probability of an event. The term “likelihood” when used in reference to the effectiveness of a patient response generally contemplates an increased probability that the symptoms of psoriasis will be lessened or decreased.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing lesional tissues, e.g. a psoriasis lesion. Skin lesions can be described as sharply demarcated, scaly, erythematous plaques often found on the extensor surfaces. Characteristic histologic findings include hyperkeratosis, parakeratosis, and acanthosis of the epidermis with dilated blood vessels and a lymphocytic infiltrate.

Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, PBMCs, tissue biopsies, skin biopsies, and the like.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered, or for a topical therapy a dose may be related to the surface area that is treated, e.g. dose/m² of skin.

Exemplary dose amounts, e.g., fixed dose amounts, for use treating an adult human by the methods of the invention include, about 0.01 mg, about 0.05 mg, about 0.1 mg, about 0.5 mg, about 1 mg, about 5 mg, about 10 mg, about 50 mg, about 100 mg, about 500 mg, or more.

Exemplary dose amounts, e.g., dose amounts for topical use treating an adult human by the methods of the invention include, about 0.01 mg/m² surface area, about 0.05 mg/m² surface area, about 0.1 mg/m² surface area, about 0.5 mg/m² surface area, about 1 mg/m² surface area, about 5 mg/m² surface area, about 10 mg/m² surface area, about 50 mg/m² surface area, about 100 mg/m² surface area, about 500 mg/m² surface area, or more.

Ranges intermediate to the above-recited ranges are also contemplated by the invention. For example, ranges having any one of these values as the upper or lower limits are also intended to be part of the invention, e.g., about 0.01 mg to about 100 mg, about 1 mg to about 10 mg, etc.

As used herein, the term “periodicity” as it relates to the administration of a substance refers to a (regular) recurring cycle of administering the substance to a subject. In one embodiment, the recurring cycle of administration of the substance to the subject achieves a therapeutic objective. The periodicity of administration of the substance may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., about 110 days to about 170 days, about 160 days to about 220 days, etc.

The “duration of a periodicity” refers to a time over which the recurring cycle of administration occurs. For example, a duration of the periodicity of administration of a substance may be may be up to about 4 weeks, up to about 8 weeks, up to about 12 weeks, up to about 16 weeks or more, up to about 20 weeks, up to about 24 weeks, up to about 28 week, up to about 32 weeks or more, during which the periodicity of administration is about once every week. For example, a duration of the periodicity may be about 6 weeks during which the periodicity of administration is about once every 4 weeks, e.g., the substance is administered at week zero and at week four.

In one embodiment, the duration of periodicity is for a length of time necessary or required to achieve a therapeutic objective, e.g., treatment, maintenance of treatment, etc. e.g., maintain a PASI 50, PASI 75, PASI 90, PASI 100 score or PGA of 0 or 1 score. Durations of a periodicity intermediate to the above-recited times are also contemplated by the invention.

As used herein, and unless otherwise specified, the terms “treat,” “treating” and “treatment” refer to an action that occurs while a patient is suffering from psoriasis, which reduces the severity of psoriasis, or retards or slows the progression of the psoriasis, or achieving or maintaining a therapeutic objective. An “effective patient response” refers to any increase in the therapeutic benefit to the patient. An “effective patient psoriasis response” can be, for example, a 5%, 10%, 25%, 50%, or 100% decrease in the physical symptoms of psoriasis.

“Treatment of or “treating” psoriasis may mean achieving or maintaining a PGA score of 0/1 or a PASI 50, PASI 75, PASI 90, or PASI 100 response score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer). “Treatment of or “treating” psoriasis may also mean achieving or maintaining a health-related quality of life (HRQOL) outcome. HRQOL outcomes include Dermatology Life Quality Index (DLQI), visual analog scales for Ps-related (VAS-Ps) and psoriatic arthritis-related (VAS-PsA) pain, Short Form 36 Health Survey Mental (MCS) and Physical (PCS) Component Summary scores, and Total Activity Impairment (TAI) scores.

“Treatment of or “treating” psoriasis may also mean achieving or maintaining a minimum clinically important difference (MCID) for any of the HRQOL outcomes provided herein, e.g., any one or combination of DLQI, VAS-Ps, VAS-PsA, MCS, PCS and TAI.

“Treatment of” or “treating” psoriasis may also mean achieving or maintaining a minimum clinically important difference (MCID) response rate for any of the HRQOL outcomes provided herein, e.g., any one or combination of DLQI, VAS-Ps, VAS-PsA, MCS, PCS and TAI. “Treatment of or “treating” psoriasis may also mean achieving or maintaining a clinically meaningful reduction in any of the HRQOL outcomes provided herein, e.g., any one or combination of DLQI, VAS-Ps, VAS-PsA, MCS, PCS and TAI.

“Treatment of or “treating” psoriasis may also mean achieving or maintaining a Nail Psoriasis Severity Index (NAPSI) score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer).

“Treatment of” or “treating” psoriasis may also mean achieving or maintaining any of the outcomes provided herein in a certain percentage of a population of subjects (e.g., in at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects).

The term “kit” as used herein refers to a packaged product comprising components with which to administer the epithelial ion channel blocker of the invention for treatment of psoriasis. The kit preferably comprises a box or container that holds the components of the kit. The box or container may be affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for use.

Formulations. The present invention provides pharmaceutically acceptable compositions which comprise a therapeutically-effective amount of at least one anti-psoriasis agent, optionally combined with one or more additional agents for treatment of psoriasis, formulated together with one or more pharmaceutically acceptable excipients. The active ingredients and excipient(s) may be formulated into compositions and dosage forms according to methods known in the art. As described in detail below, the pharmaceutical compositions of the present invention may be specially formulated for administration in solid or liquid form, including those adapted for the following: oral administration, for example, tablets, capsules, powders, granules, pastes for application to the tongue, aqueous or non-aqueous solutions or suspensions, drenches, or syrups; parenteral administration, for example, by subcutaneous, intramuscular or intravenous injection as, for example, a sterile solution or suspension. In other embodiments the formulation is provided for topical application, for example, as a lotion, cream, ointment, spray, patch, microneedle array, etc. applied to the skin.

The phrase “pharmaceutically acceptable” is employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include: ethanol, sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19.sup.th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, pregelatinized starch, sodium alginate and starch. The dissolution rate of a compacted solid pharmaceutical composition in the subjects's stomach may be increased by the addition of a disintegrant to the composition. Disintegrants include alginic acid, carboxymethylcellulose calcium, carboxymethylcellulose sodium, colloidal silicon dioxide, croscarmellose sodium, crospovidone, guar gum, magnesium aluminum silicate, methyl cellulose, microcrystalline cellulose, polacrilin potassium, powdered cellulose, pregelatinized starch, sodium alginate, sodium starch glycolate and starch.

In liquid pharmaceutical compositions of the present invention, the agent and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present invention may also contain a viscosity enhancing agent to improve the mouth-feel of the product and/or coat the lining of the gastrointestinal tract. Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Preservatives and chelating agents such as alcohol, sodium benzoate, butylated hydroxy toluene, butylated hydroxyanisole and ethylenediamine tetraacetic acid may be added at levels safe for ingestion to improve storage stability. Selection of excipients and the amounts used may be readily determined by the formulation scientist based upon experience and consideration of standard procedures and reference works in the field.

In several embodiments of the invention the anti-psoriasis agent is formulated for topical application to the skin. Various specific formulations are provided, including lotions, gels, liquids, patches, intralesional injection, and the like. A typical dose for a topical formulation in lotion or liquid form is from about 1 μl to about 100 μl to about 1 ml, to about 10 ml, applied in a lotion, cream, gel, etc. to the affected skin.

In general, the subject formulations will typically contain at least about 1 μg/ml active agent, at least about 10 μg/ml, at least about 50 μg/ml, at least about 100 μg/ml, at least about 500 μg/ml, and not more than about 100 mg/ml. In some embodiments the formulation comprises at least about 0.1 mM, at least about 0.05, at least about 1 mM, at least about 5 mM, at least about 10 mM, at least about 50 mM. The active agents of the present invention are formulated at an effective concentration within the subject formulations, meaning at a concentration that provides the intended benefit when applied topically.

The dose of active agent is as described above with respect to the surface area to be treated, where the dose may be up to about 0.01 mg/kg body weight, up to about 0.05 mg/kg body weight, up to about 0.1 mg/kg body weight, up to about 0.5 mg/kg body weight, up to about 1 mg/kg body weight, up to about 2 mg/kg body weight, up to about 5 mg/kg body weight, up to about 10 mg/kg body weight.

Administration may be every 6 hours, every 12 hours, every 24 hours, every 48 hours, every 3 days, every 4 days, every 5 days, weekly, biweekly, monthly, etc. In various of these embodiments, the therapeutically effective dose is administered on consecutive days for at least a week, at least a month, at least a year, or on as needed basis for the rest of the patient's life. The therapeutically effective dose, e.g. of anti-psoriasis agent, or pharmaceutically acceptable salt thereof, can be about 10-500 mg/day, about 50-400 mg/day, about 100-200 mg/day, or about 120-180 mg/day. anti-psoriasis agent or pharmaceutically acceptable salt thereof, can be administered to a subject at about 1-110 mg daily, 1-100 mg twice a day, 1-100 mg. every other day, as needed.

Examples are provided herein of dosages useful for treatment of an animal model. As is known in the art, in order to convert dosage from, for example, a mouse to a human, the animal dose should not be extrapolated to a human equivalent dose (HED) by a simple conversion based on body weight. The more appropriate conversion of drug doses from animal studies to human studies, uses the body surface area (BSA) normalization method. BSA correlates well across several mammalian species with several parameters of biology, including oxygen utilization, caloric expenditure, basal metabolism, blood volume, circulating plasma proteins, and renal function. See, for example, Reagan-Shaw et al. (2008) The FASEB Journal 22(3), 659-661, herein specifically incorporated by reference. The appropriate dose for a human may be roughly 1/10^{th} to 1/20^{th} of the dose for a mouse. See also, FDA guidance for Estimating the Maximum Safe Starting Dose in Initial Clinical Trials for Therapeutics in Adult Healthy Volunteers.

In some embodiments, the topical formulation comprises skin penetration enhancers. Such enhancers reversibly decrease skin barrier resistance, and include without limitation, sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes.

Topical formulations include lotions, gels, creams, etc. Such formulations may include a pharmaceutically acceptable vehicle to act as a dilutant, dispersant or carrier for the active agent(s), so as to facilitate distribution when the composition is applied to the skin. Vehicles other than or in addition to water can include liquid or solid emollients, solvents, humectants, thickeners and powders. The vehicle will usually form from 5% to 99.9%, preferably from 25% to 80% by weight of the composition, and can, in the absence of other cosmetic adjuncts, form the balance of the composition. The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

In certain specific formulations for benzamil, a labrasol (88%) based formulation may comprise benzamil (1%, w/w), span-20 (1%) and cetyltrimethylammonium bromide (CTAB) (10%). In another benzamil formulation, 1% w/w benzamil, 52 ml water, 5.2 ml glycerin, 9.7 ml rice bran oil, 4 ml caprylic aid triglyceride, 6.2 g cetearyl alcohol, 8.6 g 50/50 mixture of cetearyl glucoside and cetearyl alcohol and 3.5 g cocoa butter is combined with 48 mg benzamil in 8 ml. alcohol is mixed with 1.7 ml paraben, to which, 3 drops of wormwood oil is added.

When formulated as an emulsion, the proportion of the fatty phase may range from 5% to 80% by weight, and preferably from 5% to 50% by weight, relative to the total weight of the composition. Oils, emulsifiers and co-emulsifiers incorporated in the composition in emulsion form are selected from among those used conventionally in the cosmetic or dermatological field. The emulsifer and coemulsifier may be present in the composition at a proportion ranging from 0.3% to 30% by weight, and preferably from 0.5% to 20% by weight, relative to the total weight of the composition. When the lotions are formulated as an oily solution or gel, the fatty phase may constitute more than 90% of the total weight of the composition.

Formulations may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. Depending on their nature, these additives and adjuvants may be introduced into the fatty phase, into the aqueous phase.

Exemplary oils which may be used according to this invention include mineral oils (liquid petrolatum) and solid oils, e.g. petrolatum, plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats. Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol. Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons. Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids. Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients for lotions are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality. Powders may be incorporated into a lotion. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

An alternative formulation for topical delivery is an array of microneedles. Microneedles (MN), as used herein, refers to an array comprising a plurality of micro-projections, generally ranging from about 25 to about 2000 μm in length, which are attached to a base support. An array may comprise 10², 10¹, 10⁴, 10⁵ or more microneedles, and may range in area from about 0.1 cm² to about 100 cm². Application of MN arrays to biological membranes creates transport pathways of micron dimensions, which readily permit transport of macromolecules such as large polypeptides. In some embodiments of the invention, the microneedle array is formulated as a transdermal drug delivery patch. MN arrays can alternatively be integrated within an applicator device which, upon activation, can deliver the MN array into the skin surface, or the MN arrays can be applied to the skin and the device then activated to push the MN through the SC.

Various materials have been used for microneedles. For example, biodegradable materials into which the therapeutic agent, e.g. anti-psoriasis agent, can be incorporated are of interest. Such materials include various biodegradable or biocompatible polymers or cross-linked monomers, as known in the art. The dose of agent to be delivered will vary, and may range from at least about 1 ng/microneedle array, at least about 10 ng, at least about 0.1 μg, at least about 1 μg, at least about 10 μg, at least 0.1 mg, at least 1 mg, or more in a single array. MNs may be fabricated with a wide range of designs (different sizes and shapes) and different types (solid, hollow, sharp, or flat), and may be in-plane and/or out-of-plane.

Polymeric MNs can provide biocompatibility, biodegradability, strength, toughness, and optical clarity. To accurately produce the micro-scale dimensions of polymer MNs, a variety of mould-based techniques, such as casting, hot embossing, injection molding, and investment molding may be used, e.g. beveled-tip, chisel-tip, and tapered-cone polydimethylsiloxane (PDMS) molds. Polymeric materials of interest for fabrication include without limitation; poly (methylmetha-acrylate) (PMMA), poly-L-lactic acid (PLA), poly-glycolic acid (PGA), and poly-lactic-co-glycolic acid (PLGA), cyclic-olefin copolymer, poly (vinyl pyrrolidone), and sodium carboxymethyl cellulose. Sugars have also been used to fabricate the MNs, such as galactose, maltose, aliginate, chitosan, and dextrin. Materials may be cross-linked through ion exchange, photo-polymerization, and the like.

In other embodiments, a topical formulation is provided as a transdermal patch. Medical dressings suitable for formulation in a transdermal patch can be any material that is biologically acceptable and suitable for placing over the skin. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support is sodium carboxymethylcellulose 7H 4F, i.e. ethylcellulose.

For example, hydrocolloids (eg, RepliCare, DuoDERM, Restore, Tegasorb), which are combinations of gelatin, pectin, and carboxymethylcellulose in the form of wafers, powders, and pastes; some have adhesive backings and others are typically covered with transparent films to ensure adherence. Alginates (polysaccharide seaweed derivatives containing alginic acid), which come as pads, ropes, and ribbons (AlgiSite, Sorbsan, Curasorb), are indicated for extensive exudate and for control of bleeding after surgical debridement. Foam dressings (Allevyn, LYOfoam, Hydrasorb, Mepilex, Curafoam, Contreet) are useful as they can handle a variety of levels of exudate and provide a moist environment for healing. Those with adhesive backings stay in place longer and need less frequent changing.

In some embodiments, a transdermal patch comprises permeation enhancer, e.g. transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), and dodecyl-2-dimethylaminopropanoate (DDAIP), which may be provided at a weight/weight concentration of from about 0.1% to about 10%, usually from about 2.5% to about 7.5%, more usually about 5%.

Transdermal patches may further comprise additives to prevent crystallization. Such additives include, without limitation, one or more additives selected from octyldodecanol at a concentration of from about 1.5 to about 4% w/w of polymer; dextrin derivatives at a concentration of from about 2 to about 5% w/w of polymer; polyethylene glycol (PEG) at a concentration of from about 2 to about 5% w/w of polymer; polypropylene glycol (PPG) at a concentration of from about 2 to about 5% w/w of polymer; mannitol at a concentration of from about 2 to about 4% w/w of polymer; Poloxamer 407, 188, 401 and 402 at a concentration of from about 5 to about 10% w/w of polymer; and Poloxamines 904 and 908 at a concentration of from about 2 to about 6% w/w of polymer.

Polyvinylpyrrolidine (PVP) may also be included in a transdermal patch formulation, for example at a concentration of from about 5 wt % to about 25 weight %, about 7 wt % to about 20 wt %, about 8 wt % to about 18 wt %, about 10 wt % to about 16 wt %, about 10 wt %, about 12 wt %, about 14 wt %, about 16 wt %.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

Therapeutic formulations for treatment of psoriasis with an anti-psoriasis agent, e.g. a compound of Table 1, can be used alone or in combination with an additional agent, e.g., a therapeutic agent, said additional agent being selected by the skilled artisan for its intended purpose. For example, the additional agent can be a therapeutic agent art-recognized as being useful to treat psoriasis. The agents set forth below are illustrative for purposes and not intended to be limited. The combinations which are part of this invention can be an anti-psoriasis agent and at least one additional agent selected from the lists below. The combination can also include more than one additional agent, e.g., two or three additional agents if the combination is such that the formed composition can perform its intended function.

Additional therapeutic agents include, without limitation, methotrexate, 6-MP, azathioprine sulphasalazine, mesalazine, olsalazine chloroquinine/hydroxychloroquine, pencillamine, aurothiomalate (intramuscular and oral), azathioprine, colchicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, etc., phosphodiesterase inhibitors, adensosine agonists, antithrombotic agents, complement inhibitors, adrenergic agents, agents which interfere with signaling by proinflammatory cytokines such as TNF.alpha. or IL-1 (e.g. IRAK, NIK, IKK, p38 or MAP kinase inhibitors), IL-1.beta. converting enzyme inhibitors (e.g., Vx740), anti-P7s, p-selectin glycoprotein ligand (PSGL), TNF.alpha. converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG and p55TNFRIgG, sIL-1RI, sIL-1RII, slL-6R, soluble IL-13 receptor (slL-13)) and anti-inflammatory cytokines (e.g. IL-4, IL-10, IL-11, IL-13 and TGFβ). In some embodiments the dose of the additional therapeutic agent when co-formulated with an anti-psoriasis agent is lower than the conventional dose. In some embodiments, anti-psoriasis agent is co-formulated with a glucocorticoid.

Treatment with an anti-psoriasis agent can also be combined with PUVA therapy. PUVA is a combination of psoralen (P) and long-wave ultraviolet radiation (UVA) that is used to treat many different skin conditions. In still another embodiment, the compositions of the invention are administered with excimer laser treatment for treating psoriasis.

Treatment for psoriasis often includes a topical corticosteroids, vitamin D analogs, and topical or oral retinoids, or combinations thereof. In one embodiment, an anti-psoriasis agent is administered in combination with or the presence of one of these common treatments.

The composition can be packaged in any suitable container to suit its viscosity and intended use. The invention accordingly also provides a closed container containing a therapeutically acceptable composition as herein defined.

The pharmaceutical compositions of the invention may include a “therapeutically effective amount” or a “prophylactically effective amount”. A “therapeutically effective amount” refers to an amount effective, at dosages and for periods of time necessary, to achieve the desired therapeutic result. A therapeutically effective amount may vary according to factors such as the disease state, age, sex, and weight of the individual, and the ability to elicit a desired response in the individual. A therapeutically effective amount is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

In one embodiment, the dose is administered to the subject upon a flare of psoriasis. In another embodiment, the dose is administered to the subject prior to a flare of psoriasis.

The flare of psoriasis may be monitored by determining a subject's Psoriasis Area and Severity Index (PAST), e.g., PASI 100 response, PASI 90 response, PASI 75 response, PASI 50 response, the PASI response of a single body region, two body regions, three body regions, or four body regions, e.g., trunk, lower extremities, upper extremities, or head and neck. Alternatively, the flare of psoriasis may be monitored by determining a subject's Physician's Global Assessment (PGA) rating.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### Methods of Use

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

A composition comprising an effective dose of an anti-psoriasis agent, optionally combined with additional therapeutic agents, is provided to an individual with psoriasis. The administration can be oral, parenteral, topical, etc. In some embodiments topical is preferred. The dosing and periodicity of administration is selected to provide for therapeutic efficacy.

In one embodiment, the subject achieves at least a PGA score of 0 or 1. In one embodiment, the subject achieves at least a PASI 75 response. In one embodiment, the subject achieves at least a PASI 90 response. In one embodiment, the subject achieves at least a PASI 100 response. In one embodiment, the subject maintains the PGA score of 0 or 1 during treatment. In one embodiment, the subject maintains the PASI 75 response during treatment. In one embodiment, the subject maintains the PASI 90 response during treatment.

In one embodiment, the subject achieves a PGA score of 0 or 1, e.g., by about week 12. In one embodiment, the subject achieves at least a PASI 75 response, e.g., by about week 12. In one embodiment, the subject achieves at least a PASI 90 response, e.g., by about week 12. In one embodiment, the subject achieves at least a PASI 100 response, e.g., by about week 12.

In one embodiment, the subject maintains the PGA score of 0 or 1 through the duration of treatment. In one embodiment, the subject maintains the PASI 75 response through the duration of treatment. In one embodiment, the subject maintains the PASI 90 response through the duration of treatment.

In certain embodiments of the foregoing aspects, the subject or population of subjects achieves (i) an improvement in a Dermatology Life Quality Index (DLQI) score or mean Dermatology Life Quality Index (DLQI) score of at least about −9; (ii) an improvement in a Short Form 36 Health Survey Physical Component Summary (PCS) score or mean Physical Component Summary (PCS) score of at least about 2; (iii) an improvement in a Short Form 36 Health Survey Mental Component Summary (MCS) score or mean Short Form 36 Health Survey Mental Component Summary (MCS) score of at least about 4; (iv) an improvement in a visual analog scale score or mean visual analog scale score for psoriasis-related pain (VAS-Ps) of at least about −25; (v) an improvement in a visual analog scale score for psoriatic arthritis-related pain (VAS-PsA) or mean visual analog scale score for psoriatic arthritis-related pain (VAS-PsA) of at least about −32; and/or (vi) a minimum clinically important difference (MCID) response rate for psoriasis-related pain (VAS-Ps) of at least about 60%.

In various aspects, the invention is directed to a method of treating psoriasis in a population of subjects, wherein the population of subjects achieves (i) a minimum clinically important difference (MCID) response rate for Dermatology Life Quality Index (DLQI) of at least about 70% by about week 12; (ii) a minimum clinically important difference (MCID) response rate for Dermatology Life Quality Index (DLQI) of at least about 81% by about week 52; (iii) a minimum clinically important difference (MCID) response rate for Total Activity Impairment (TAI) of at least about 45% by about week 12; and/or (iv) a minimum clinically important difference (MCID) response rate for Total Activity Impairment (TAI) of at least about 57% by about week 52. In one embodiment, the antibody, or antigen-binding portion thereof, is administered once every four weeks. In another embodiment, the antibody, or antigen-binding portion thereof, is administered once every 12 weeks.

In certain embodiments of the various aspects of the invention, the subject achieves a Nail Psoriasis Severity Index (NAPSI) score of about 2.1 or less. In certain embodiments, the subject achieves a Nail Psoriasis Severity Index (NAPSI) score of about 2.1 or less by about week 24. In related embodiments of the various aspects of the invention, the subject achieves a Nail Psoriasis Severity Index (NAPSI) score of about 1.2 or less. In certain embodiments, the subject achieves a Nail Psoriasis Severity Index (NAPSI) score of about 1.2 or less by about week 52.

### EXPERIMENTAL

### Example 1

### Computational Drug Repositioning Identifies Novel Agents for Treatment of Psoriasiform Dermatitis

Psoriasis is characterized by a pathologic inflammatory interplay between skin cells and immunocytes resulting in disfiguring cutaneous lesions and systemic inflammation. Immunosuppression is used to target the inflammatory component, however, these drugs are often expensive and associated with side effects. To identify novel targets potentially impacting epidermal pathways, we carried out a non-biased informatics screen to identify drug entities that could be repurposed for use in psoriasis, enriching for inverse signatures of psoriatic skin. We predicted that benzamil and other agents could reverse psoriasiform hyperplasia and inflammation in both murine and reconstructed human psoriasis patient skin. Based on targeted CRISPR- and proteomic screen, we propose that benzamil targets sodium exchangers in keratinocytes, perturbing pathogenic interactions between the small GTPase Rac1 and its adaptor NCK1. Inhibition of sodium channels, or preventing excess Rac1-NCK1 binding, rescues psoriasis-associated signaling pathways in patient keratinocytes and can effectively be accomplished through topical delivery of benzamil. We present novel, cost-effective, potent immune-sparing therapies for psoriasis.

We applied a rank-aggregation algorithm to meta-analyze disease-specific gene-expression signatures to derive a disease-PRL that can be systematically compared to drug-PRLs for drug repositioning in psoriasis. Meta-analysis of publicly available datasets from ˜200 psoriasis patients were compared to gene-expression signatures yielding candidate drugs. By testing this with benzamil in primary human patient cells, a psoriasis mouse model and an in vivo model of primary reconstructed human psoriatic skin, we uncovered a mechanism whereby benzamil reverses psoriasis-associated signaling pathways and reverses the psoriasis skin phenotype, through an immune-sparing, epidermal targeted approach.

### Results

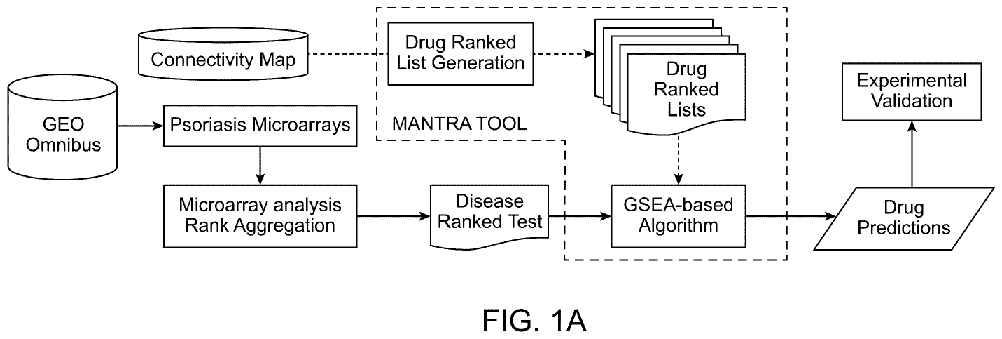

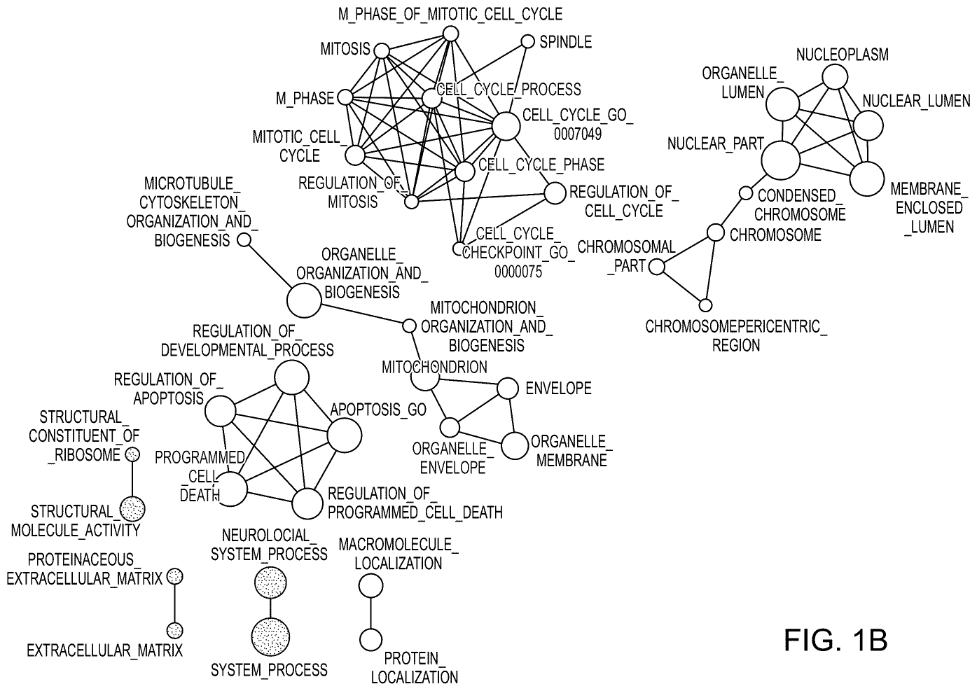

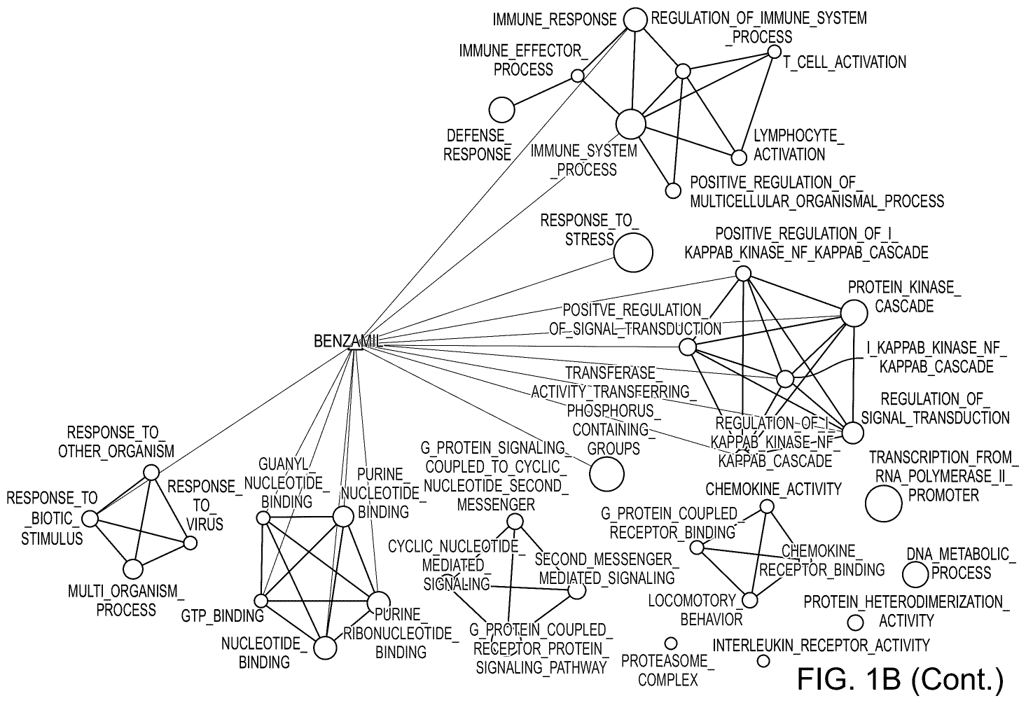

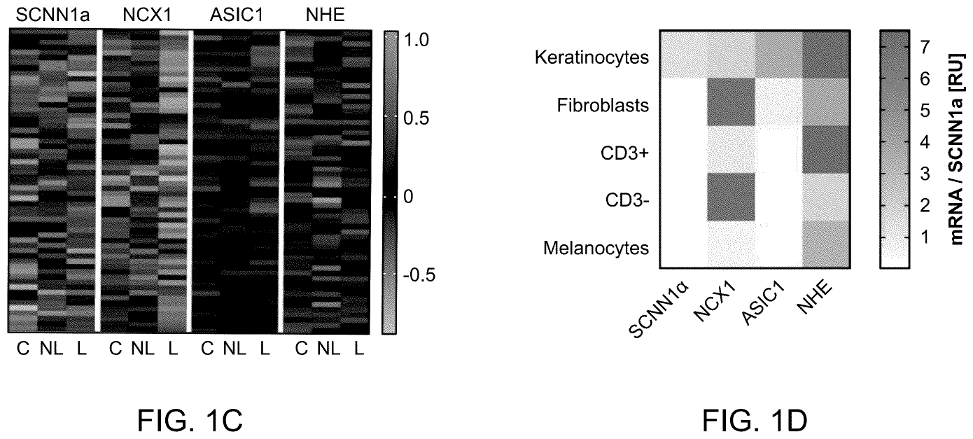

Drug repositioning reveals benzamil as a potential therapy for psoriasis. We hypothesized that drugs yielding opposite gene expression signatures to those found in psoriatic lesional skin could be therapeutic. To test this, genome-wide gene expression data from patients with psoriasis were obtained from the NCBI Gene Expression Omnibus (GEO, Table 2). Drug predictions were made using the MANTRA online tool (FIG. 1A). The MANTRA method produces total enrichment scores (TES) as a statistical measure of similarity between two gene-signatures that range between 0 and 2, with 0 representing two identical gene lists. We used a TES of 0.8 as a threshold of significance to identify drug-disease connections that were statistically significant as demonstrated previously. At this level of significance, 13 drug candidates were identified, 7 of which are commercially available, and 5 with vendor-only names (Table 1). The 5 top ranking candidates included quinostatin, benzamil, bromocriptine, trifluoperazine and sirolimus, which had direct or indirect evidence supporting their use in psoriasis, lending support to the validity of our computational repositioning approach.

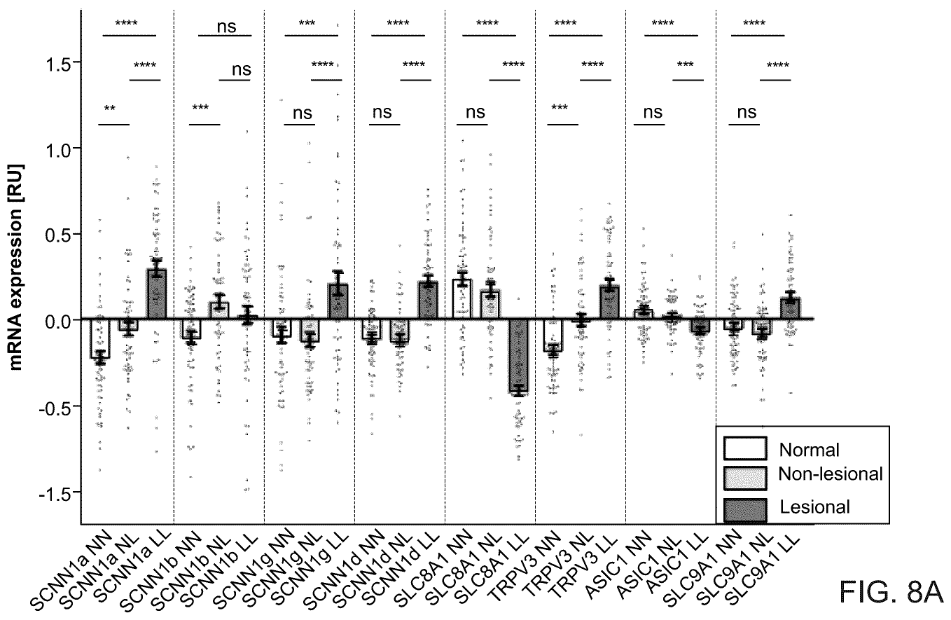

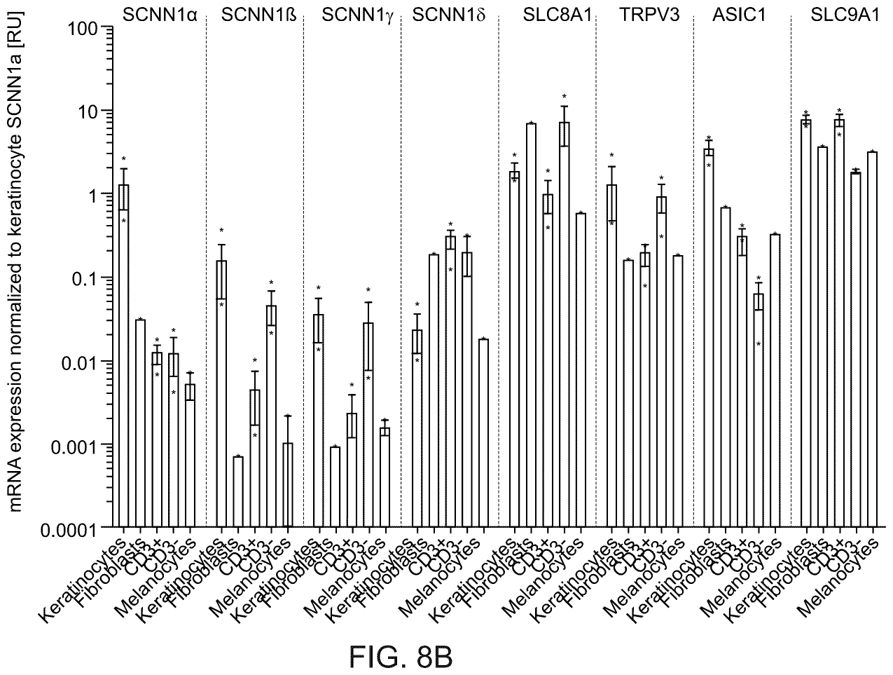

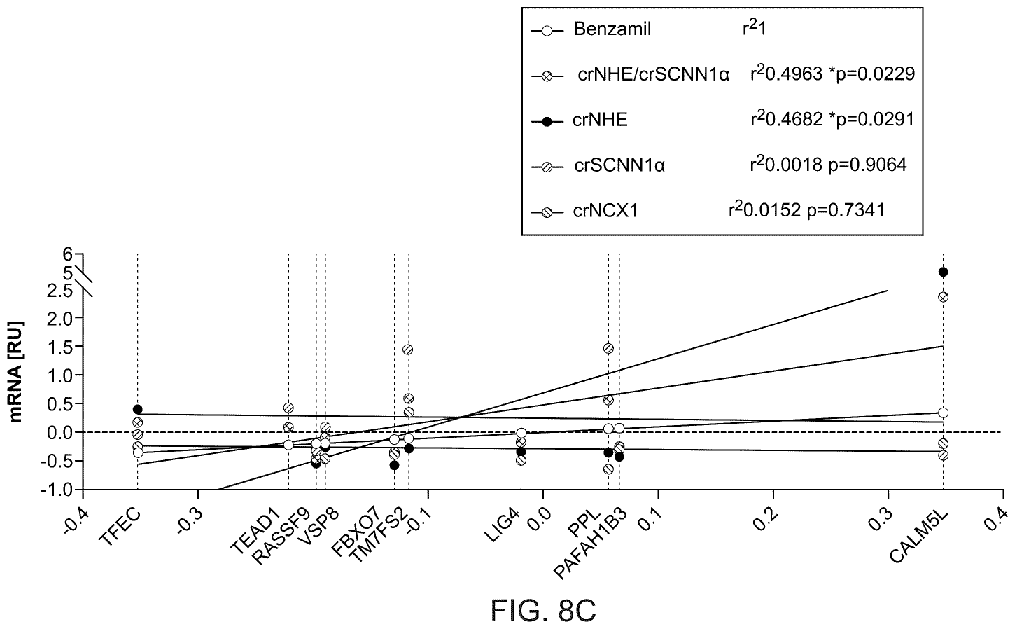

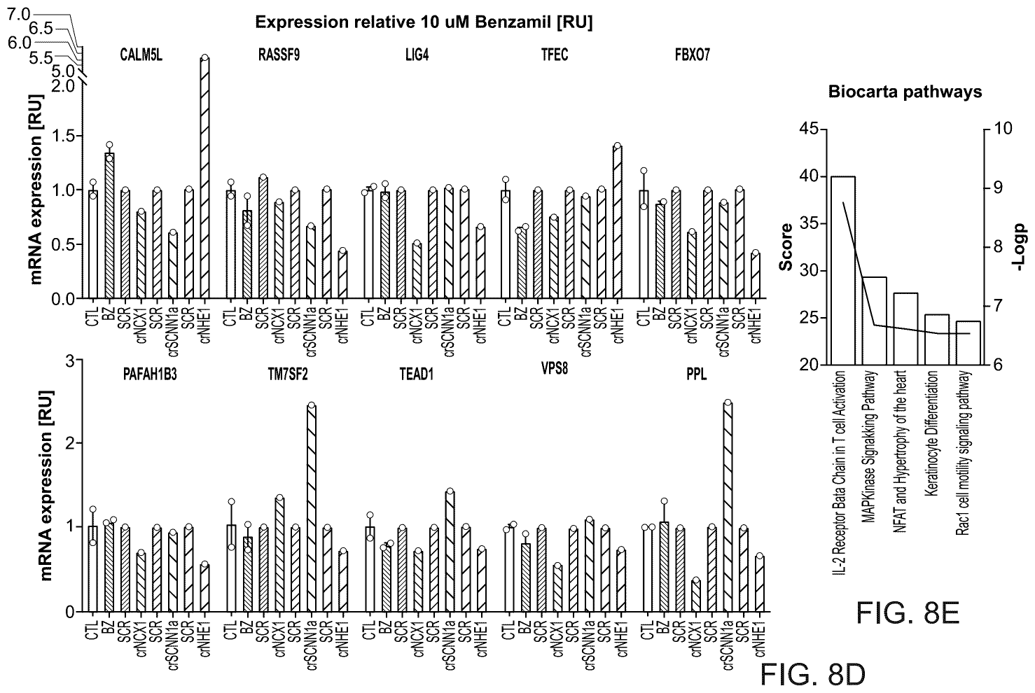

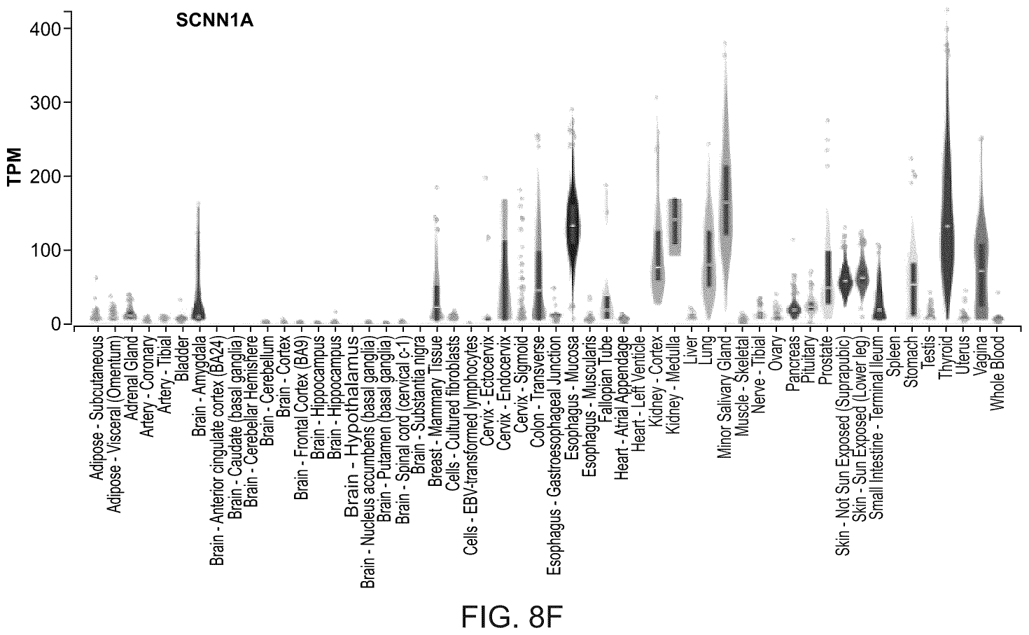

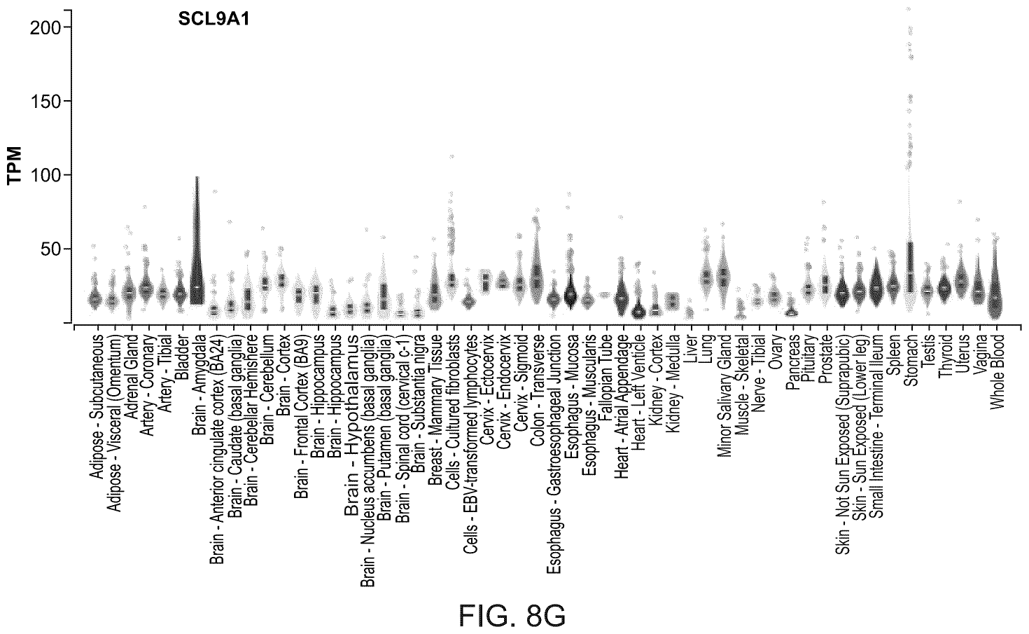

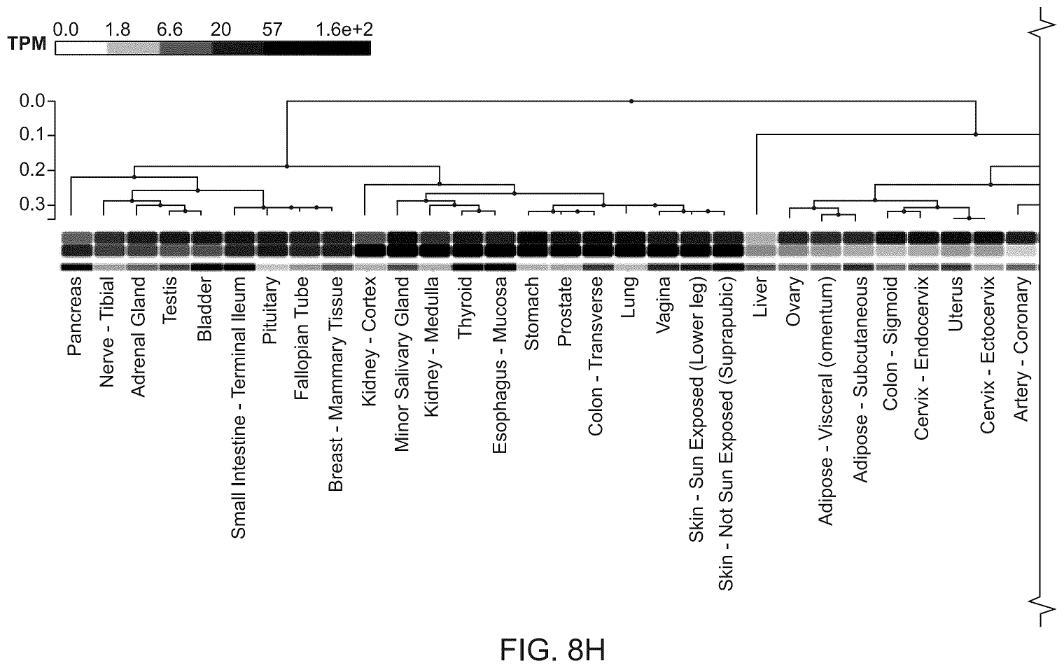

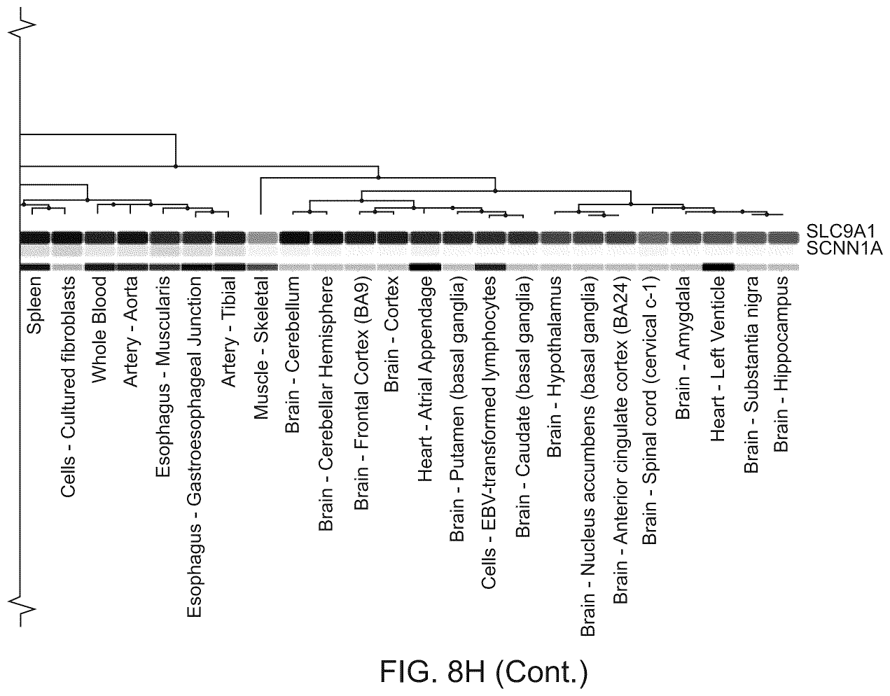

Of these candidates, benzamil was chosen as a promising drug for experimental validation, on the basis of a novel, untested mechanism of action, and its prior approval and use in patients. To identify biological processes in psoriasis likely targeted by benzamil, we performed gene-set enrichment analysis on the psoriatic signature to identify major Gene Ontology gene-sets enriched in psoriasis samples, compared to normal skin. We then identified which gene-sets significantly overlapped with the benzamil signature gene-set (FIG. 1B). GSEA-enriched gene-sets with a FDR≤0.001 were then visualized using the Enrichment Map tool plugin in Cytoscape. Statistically significant connections with a p-value ≤0.10 (hypergeometric test) between the benzamil gene-set and GO gene-sets were visualized. 16 out of the 78 GO gene-sets enriched in the psoriasis signature were significantly overlapping with the benzamil gene-set. To assess ontology pathways associated with the paired benzamil-psoriasis signature, we used BIOCARTA and identified the highest enriched pathways to encompass T-cell activation (ranked 1), MAPK signaling pathway (2), keratinocyte differentiation (4) and Rac1 signaling (5) (FIG. 8E).

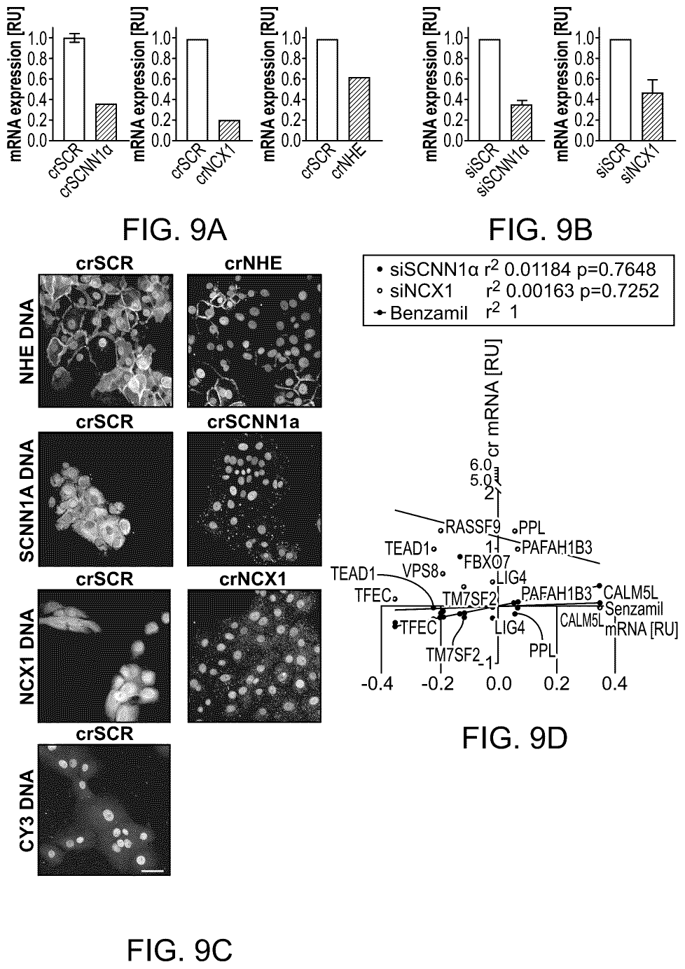

CRISPR screen identifies target of benzamil in primary cells To elucidate the molecular target of benzamil we first queried psoriasis datasets for differential expression of benzamil targets, the SCNN1a subunit representing the epithelial sodium channel (ENAC): the sodium calcium exchanger (NCX1): the Transient Receptor Potential Cation Channel Subfamily V Member 3 (TRPV3), the Acid Sensing Ion Channel Subunit 1 (ASIC) and the sodium hydrogen exchanger (NHE1) (FIG. 1C, FIG. 8A). SCNN1α, TRPV3 and NHE1 expression were significantly (p<0.0001) induced in psoriatic lesional skin compared to control skin, while NCX1 was significantly repressed. To verify that these targets were expressed in keratinocytes and/or immunocytes, we compared mRNA expression of these benzamil targets by quantitative reverse transcription PCR (RT-qPCR) in keratinocytes, fibroblasts, melanocytes, CD3+ and CD3− PBMCs, and confirmed enriched expression in epithelial cells using the gene expression portal (FIG. 8 F-G). We found that NHE had the highest expression in keratinocytes and CD3+ immunocytes, whereas ENAC was predominantly expressed in keratinocytes (FIG. 1D, FIG. 8B). To assess potential targets underlying the benzamil gene-expression signature in psoriasis, we selected 10 target genes that had high expression in keratinocytes and queried the potential targets with high expression in keratinocytes (SCNN1□), moderate in both keratinocytes and T-cells (NCX1), or high expression in both keratinocytes and T-cells (NHE1) through a targeted CRISPR-Cas9 screen. After validating mRNA and/or protein in knockout cells (FIG. 9A-C) we found that crNHE1, but not crSCNN1α or crNCX1 significantly mimicked the signature of benzamil (FIG. 8C). However, combining the signature of crNHE and crSCNN1α (but not crNHE1 and crNCX1 or crNCX1 and crSCNN1α) resulted in more overlapping signature (r² 0.4963, p<0.05) than just crNHE1 compared to benzamil. Although this goodness of fit demonstrates deviance from baseline rather than intervariable comparison, this implicates resemblance between sodium exchangers perturbation and transcriptional alterations by benzamil in keratinocytes. To verify a lack of correlation with NCX1 or SCNN1^{α}, we repeated the SCNN1α and NCX1 loss of function screen using siRNAs, with similar results (FIG. 9D). Altogether these combined results demonstrate that the ion channel inhibitor benzamil had a signature anti-correlating with that of psoriasis, that GSEA GO-terms implicate keratinocyte and T cell signaling, and the sodium exchangers ENAC and NHE1 as significantly induced in psoriatic lesional skin including keratinocytes (ENAC, NHE1) and T-cells (NHE), and that perturbing sodium exchangers in primary cells mimicked the benzamil signature.

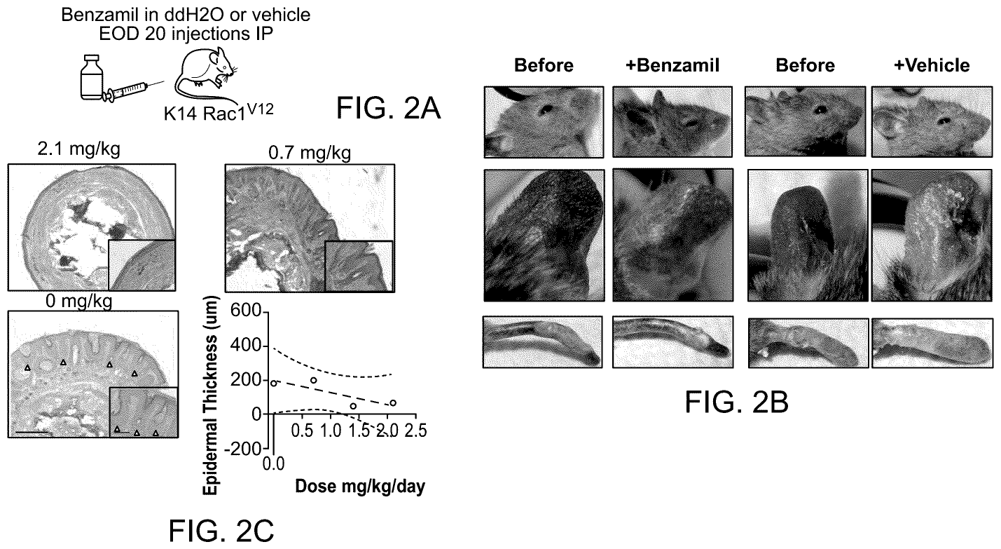

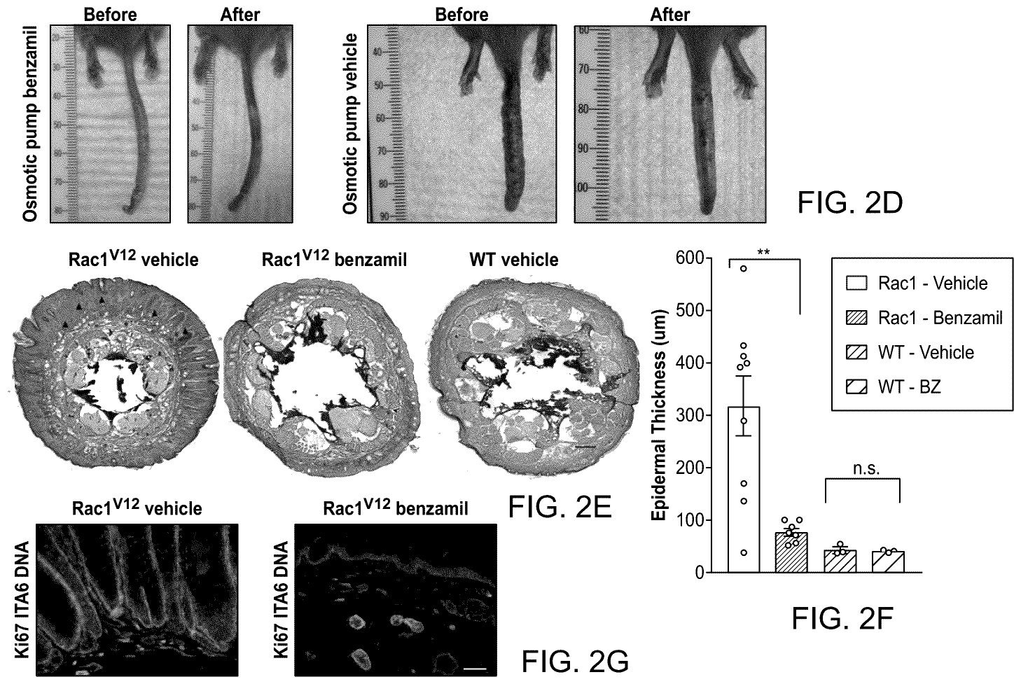

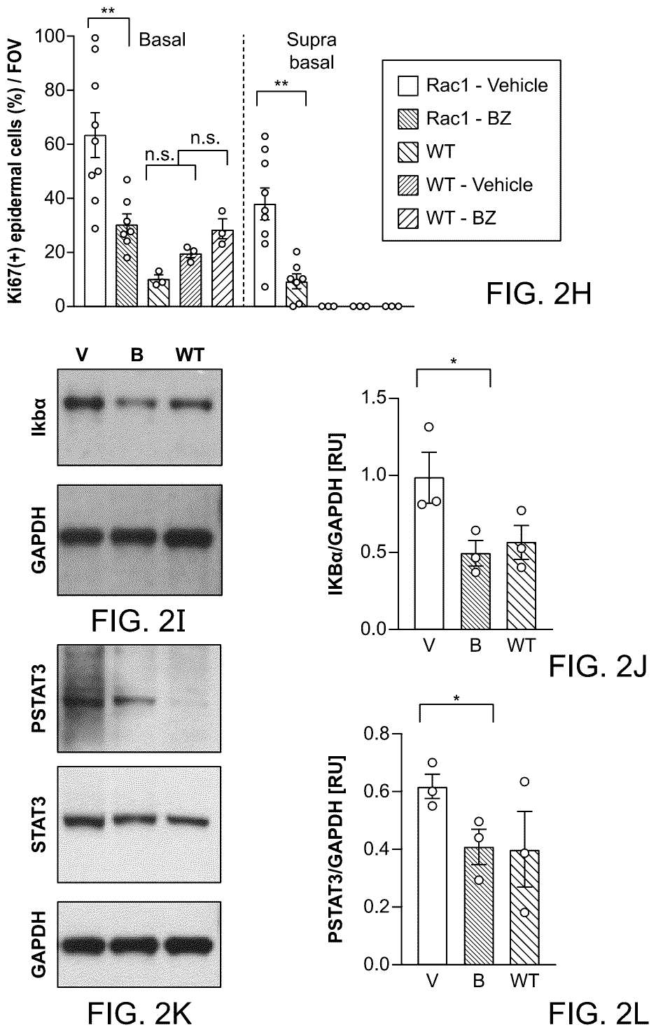

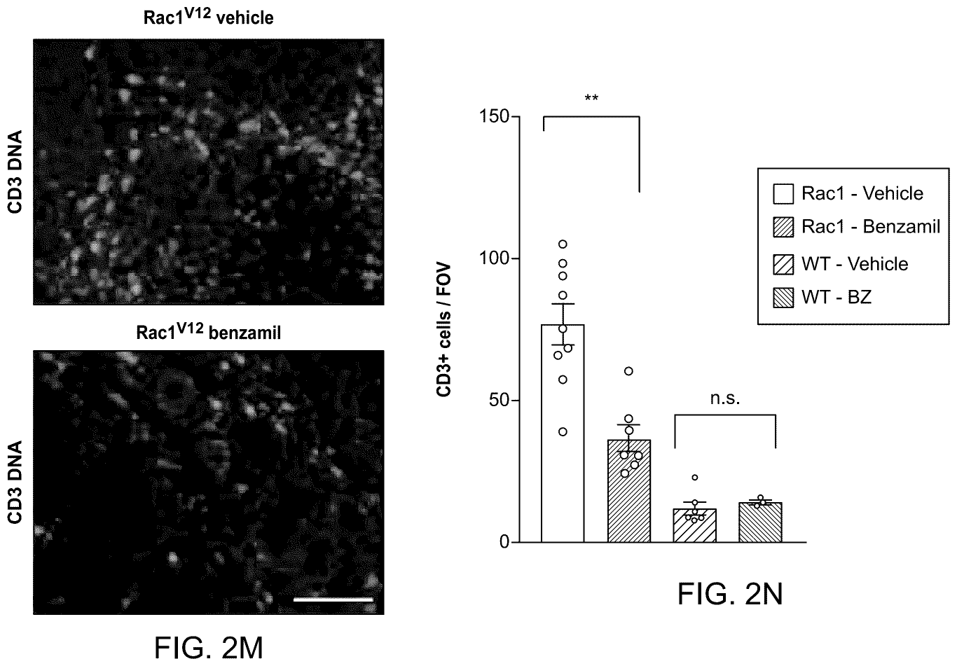

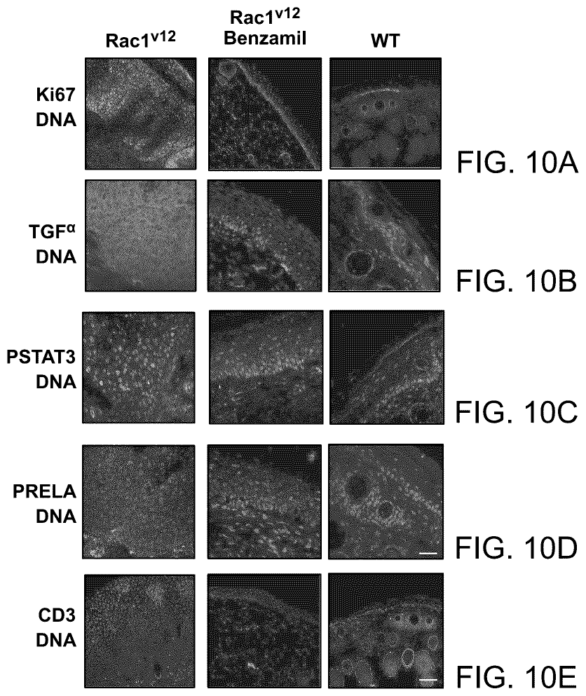

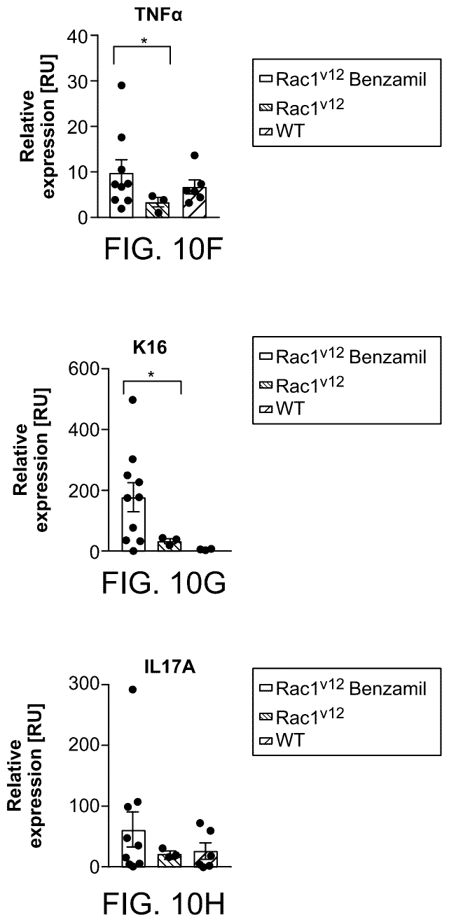

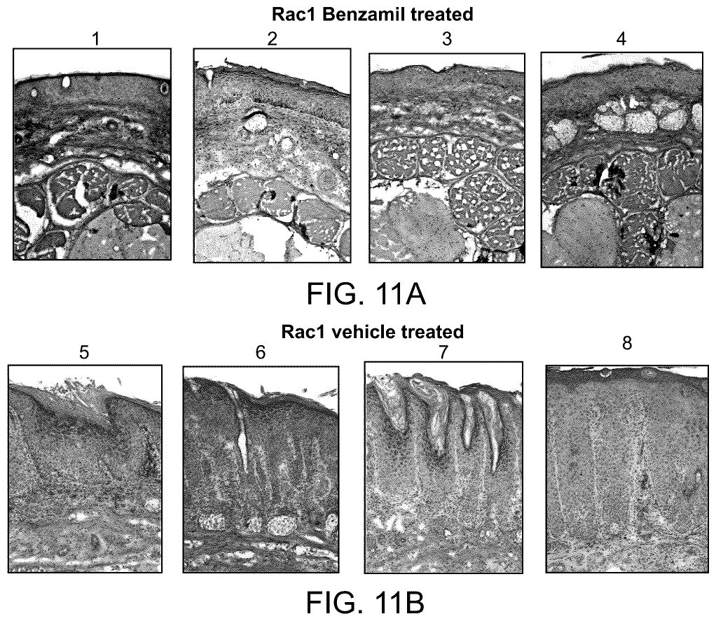

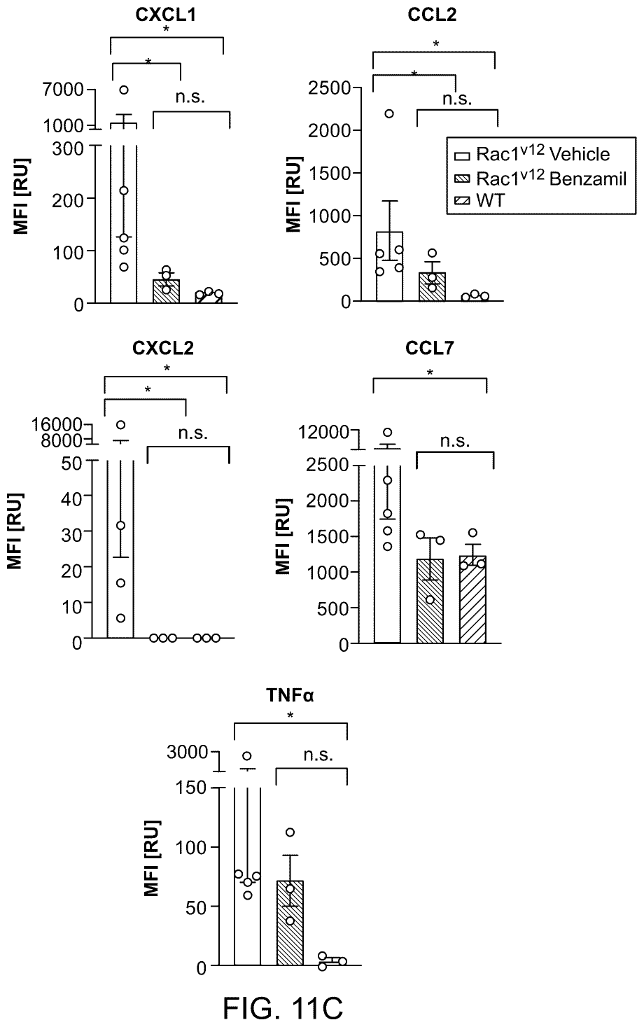

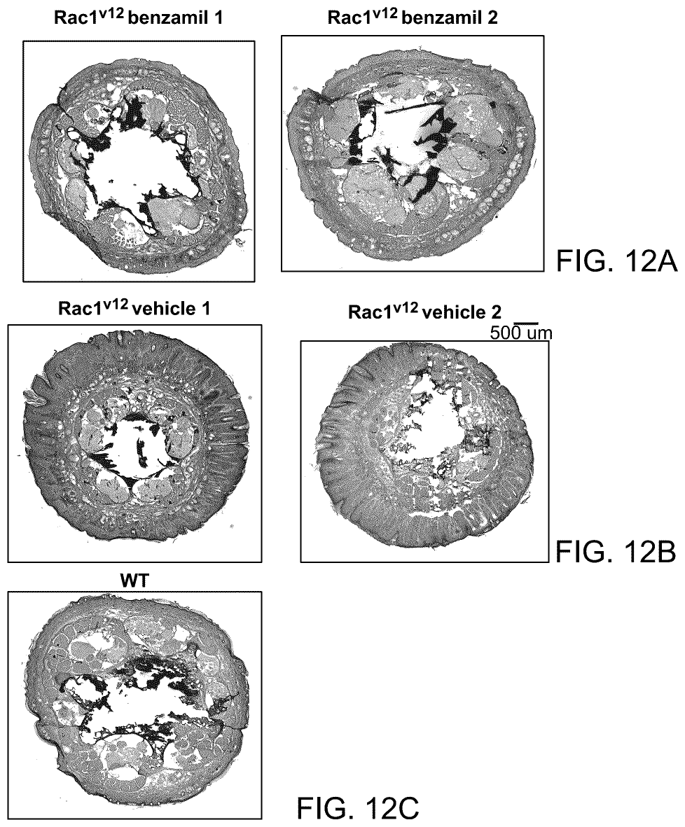

Benzamil reverses the disease phenotype of a psoriasis model in vivo Given the imputed effect of benzamil on psoriasis, we utilized a Rac1^{V12} transgenic mouse model of psoriasis for in vivo validation. As an initial experiment, we administered benzamil or saline control systemically through intraperitoneal injections (FIG. 2A). Benzamil treated mice exhibited marked signs of improvement, including reduced scaling, erythema and edema on muzzle and ears as well as healing of tail lesions (FIG. 2B). Benzamil-treated skin exhibited reduced epidermal hyperproliferation in a dose dependent fashion (FIG. 2C), thereby reversing the epidermal psoriasis phenotype. Treated mice exhibited reduced Ki67 (FIG. 10A), TGFα (FIG. 10B), phospho-STAT3 (FIG. 10C), phospho-relA (FIG. 10D) expression and reduced CD3+ T cell infiltration (FIG. 10E). Additionally, we found reduced mRNA expression of both keratin 16 and TNFα by RT-qPCR (FIG. 10. FH). To validate these preliminary findings in a larger cohort and to control for benzamil release over time, Rac1V12 mice were implanted with osmotic pumps containing benzamil or vehicle control for 28 days. Constant release rate of benzamil significantly reduced epidermal hyperplasia compared to vehicle, despite a 30% shorter treatment duration compared to IP injected mice (FIG. 2D-F, FIG. 11A-B, FIG. 12A-C). This was accompanied by a significant reduction in suprabasal proliferation (FIG. 2G-H). Whole skin lysates from mice treated with benzamil or vehicle showed normalized NFκB expression (FIG. 21-J) as well as reduced STAT3 signaling (FIG. 2K-L). and CD3+ T-cell skin infiltration (FIG. 2M-N). Thus, these results demonstrate the efficacy of benzamil in a validated in vivo murine model of human psoriasis.

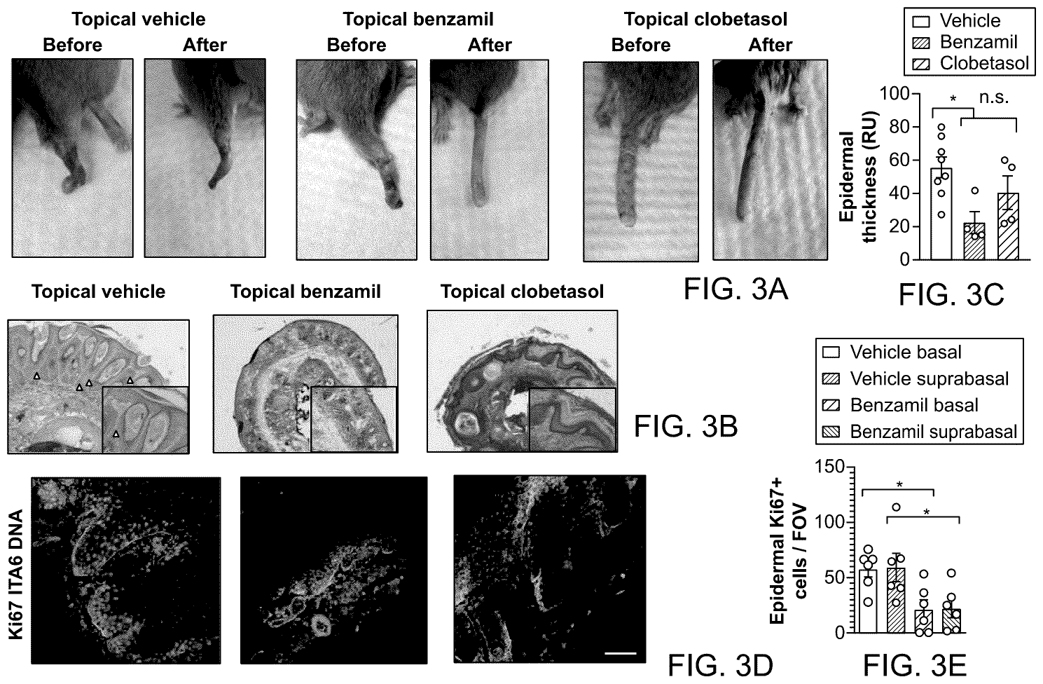

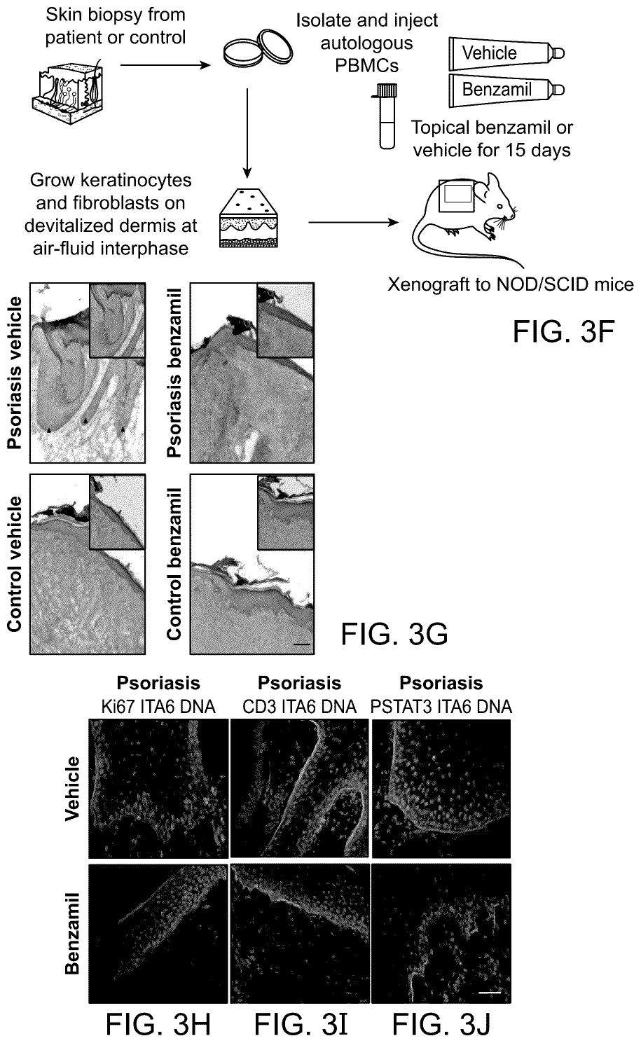

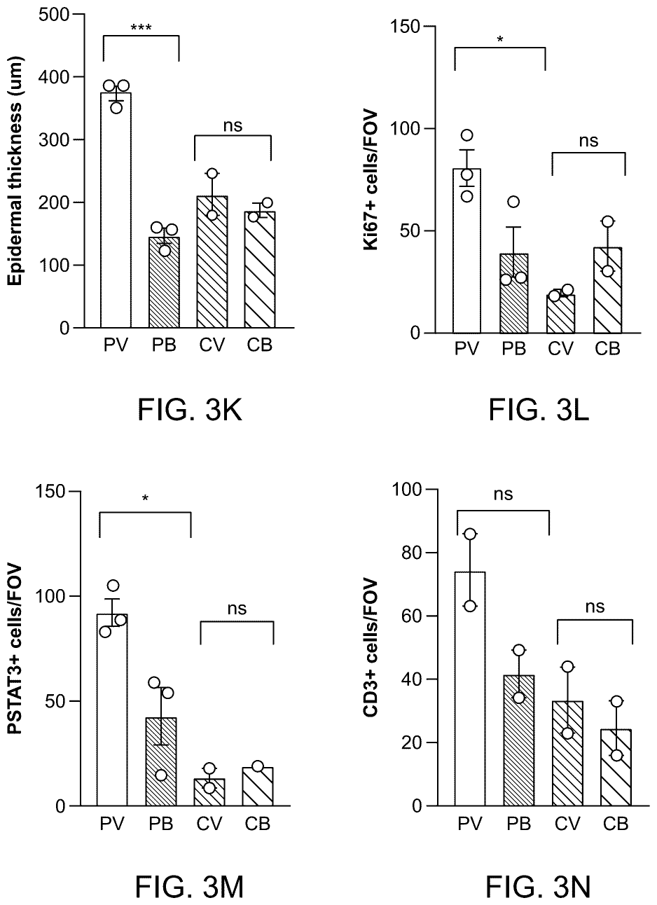

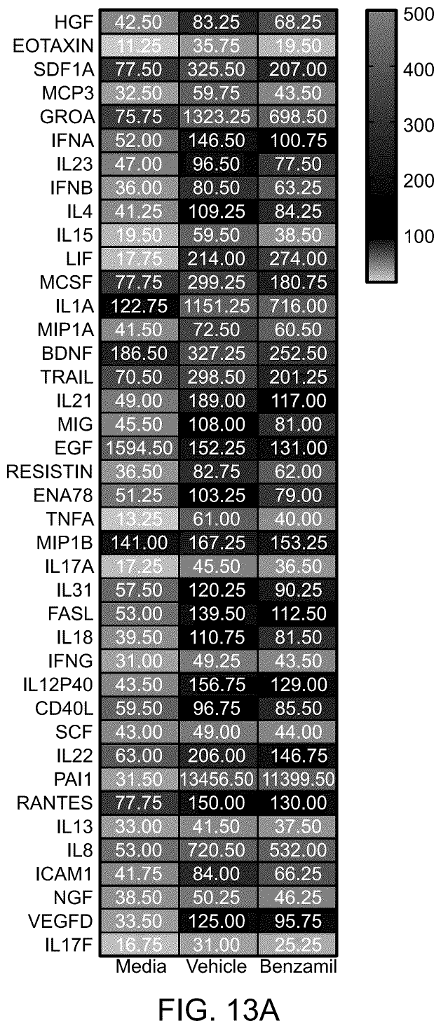

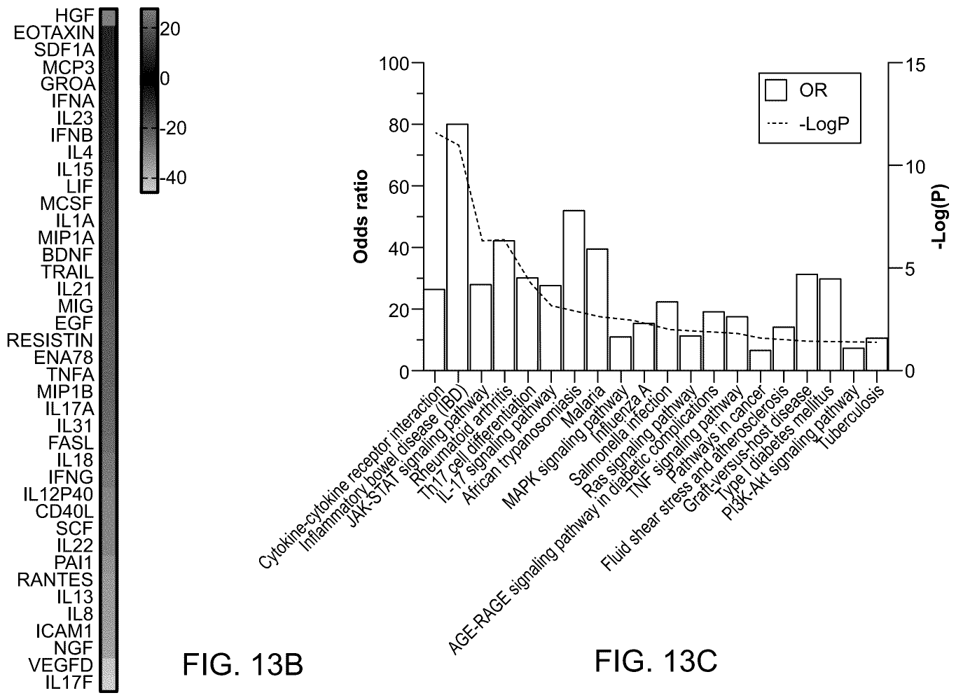

Benzamil is a potent topical therapy in mouse and reconstructed human psoriatic skin. To understand how systemic benzamil affects cytokine production in skin, we performed multiplex Luminex assays on the skin of benzamil-treated Rac1V12 mice. We found a significant reduction in CXCL1, CCL2, CXCL2, CCL7 as well as TNFα (FIG. 11C). Since benzamil inhibited both keratinocyte hyperproliferation and cutaneous chemotactic cytokine production, we hypothesized that topical benzamil (TB) could be effective. Indeed TB significantly reduced psoriasiform inflammation (FIG. 3A), as well as epidermal hyperplasia (FIG. 3B-C) and hyperproliferation (basal and supra-basal Ki67) compared to vehicle control (FIG. 3D-E) with a similar reduction in epidermal thickness as the potent topical corticosteroid clobetasol. We next aimed to determine whether TB could prevent the psoriasiform hyperplasia of reconstructed human skin from primary psoriatic cells in a xenograft model. To achieve this, organotypic skin equivalents were produced using primary human psoriasis or control keratinocytes, and grafted on to immunodeficient NOD/SCID mice. After bandage removal and intradermal injection of autologous PBMCs, xenografts were treated with TB or vehicle control (FIG. 3F). Whereas vehicle-treated patient grafts showed pronounced psoriasiform hyperplasia, TB-treated grafts showed thickness that was comparable to normal control graft skin (FIG. 3H). Immunofluorescence analysis using confocal microscopy showed reduced levels of suprabasal Ki67, CD3+ cell infiltration and phospho-STAT3 expression (FIG. 3H-J). TB reduced epidermal thickness, Ki67+ epidermal cells and epidermal phospo-STAT3, to levels comparable to control grafts (FIG. 3K-N). In parallel, we performed luminex cytokine assays on primary psoriasis cultures from donors, and found that cytokines involved in cytokine-cytokine receptor signaling (IL21;IL22;IL1A;IL31;IFNG;IL15;IL13;LIF;IL18;IL17F;NGF;IL17A),Th17 differentiation and Th17 signaling (IL13: IL21;IL22;IFNG;IL17F;IL17A as well as JAK-STAT-signaling (IL21;IL22;IFNG;IL15;EGF;IL13;LIF) were significantly reduced by benzamil (FIG. 13A-C). Collectively, these data demonstrate that topical benzamil effectively inhibited psoriasiform hyperplasia and psoriasis-related signaling pathways in vivo, both in transgenic mice and primary regenerated human psoriatic skin.

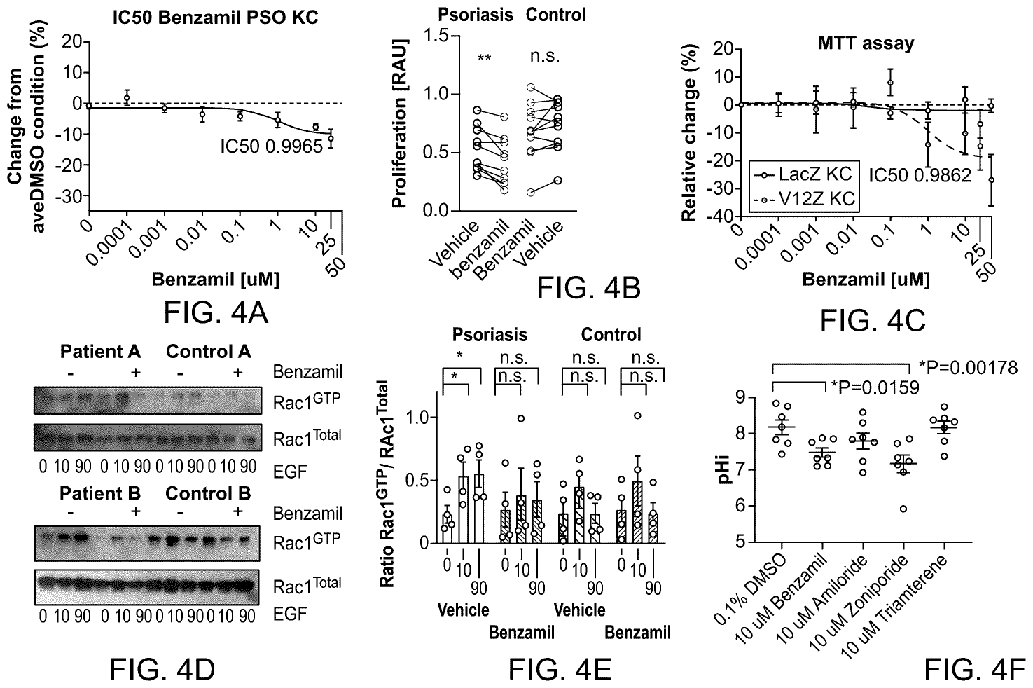

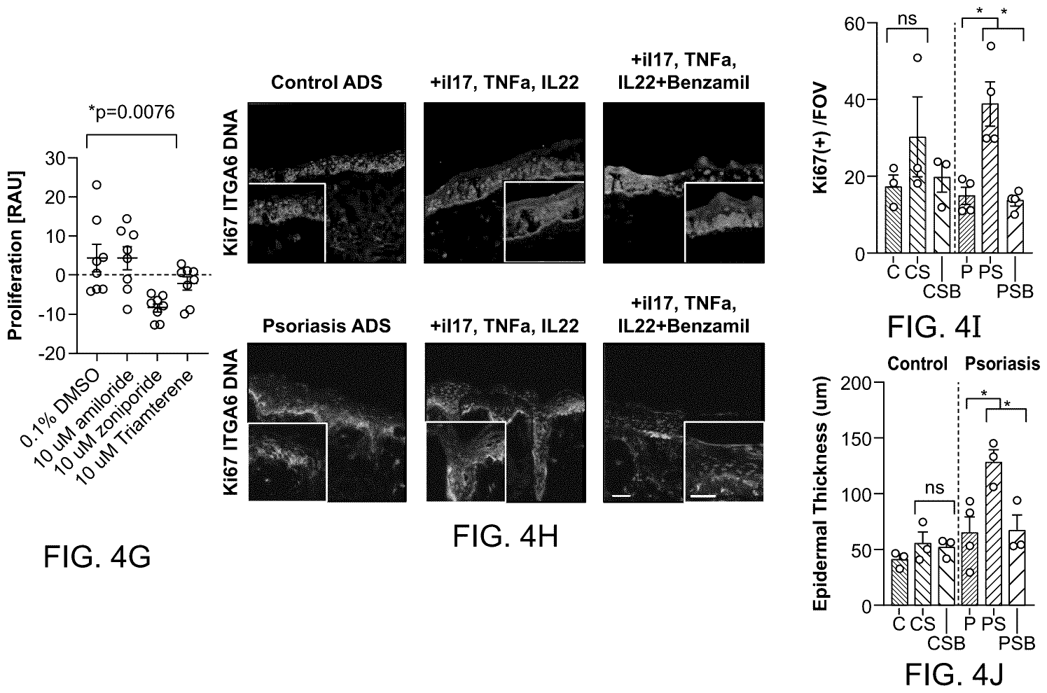

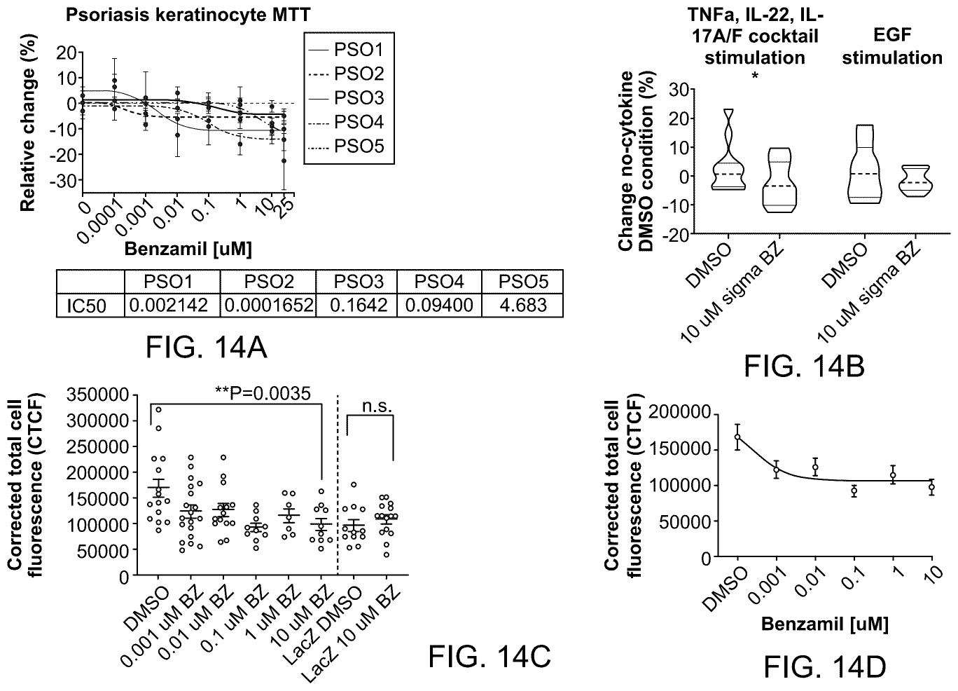

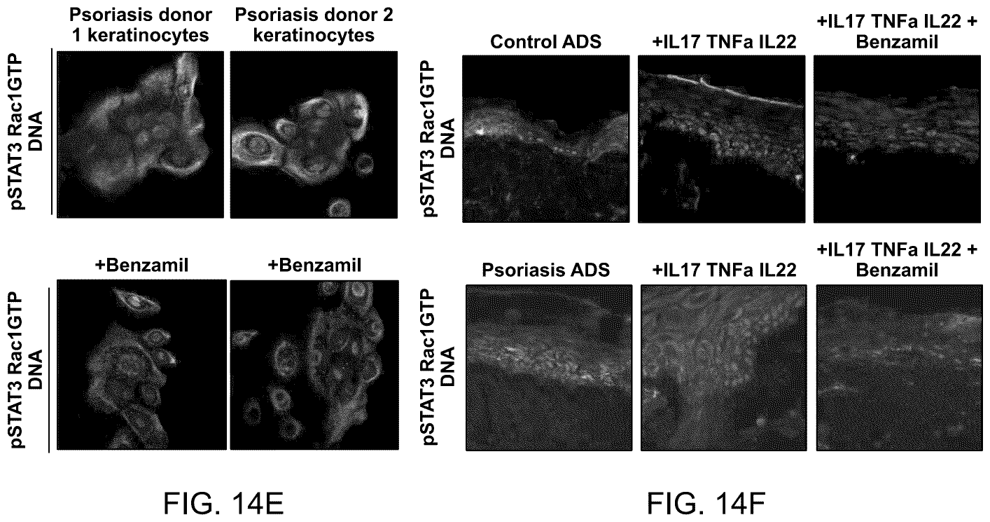

Benzamil inhibits psoriatic signaling pathways in primary human cells. Based on the effect of benzamil in reconstructed primary human psoriatic skin comprising of both epidermal primary cells as well as immunocytes, we hypothesized benzamil exhibited its inhibitory effect on psoriasiform hyperplasia through effects on keratinocytes, immunocytes, or both. We assayed benzamil's effect on keratinocyte proliferation in vitro using cells from 5 psoriasis donors, and found benzamil inhibited proliferation with a half maximal inhibitory concentration (IC50) of 0.99 μM (FIG. 4A, FIG. 14A). In 9 paired primary psoriasis patient cultures, there was a significant reduction in proliferation, not seen in 11 paired normal primary control cultures (FIG. 4B) suggesting benzamil has considerable safety in normal human skin. Further, treating psoriatic keratinocytes stimulated with a cocktail of IL22, IL17 and TNFα with benzamil demonstrated a significant reduction in cytokine-driven proliferation after 24 hours (FIG. 14B).

Given this differential effect between psoriatic and normal keratinocytes, and taken our in silico analysis and effect of benzamil in a Rac1-driven mouse model of psoriasis into account, we hypothesized that the benzamil inhibitory effect in psoriatic keratinocytes were driven by Rac1. Thus, we overexpressed a Rac1V12 mutant in normal keratinocytes and found that these keratinocytes were differentially inhibited by Benzamil compared to LacZ transduced control cells (FIG. 4C). We previously demonstrated that the small GTPase Rac1 is constitutively activated in the GTP bound state in human psoriatic epidermis and that transgenic mice with a Rac1 mutant (Rac1G12V) under a keratin 14 promoter exhibit the major clinical, histological and biochemical hallmarks of human psoriasis, and identified a key role for psoriatic epidermal Rac1 in immunocyte activation. As our in silico results suggested a role for benzamil in T-cell activation and Rac1 signaling, we hypothesized that this effect might be secondary to perturbation of keratinocyte Rac1 activity. To test this hypothesis, we stimulated psoriatic keratinocytes with EGF (correlates well with other stimulants such as IL17, IL22 and TNFα as a proxy for Rac1 activation) in the presence or absence of benzamil (10 μM), and identified, using PAK-pulldown beads to pull down active Rac1, that benzamil reduced Rac1^{GTP} (p<0.05) (FIG. 4D-E).

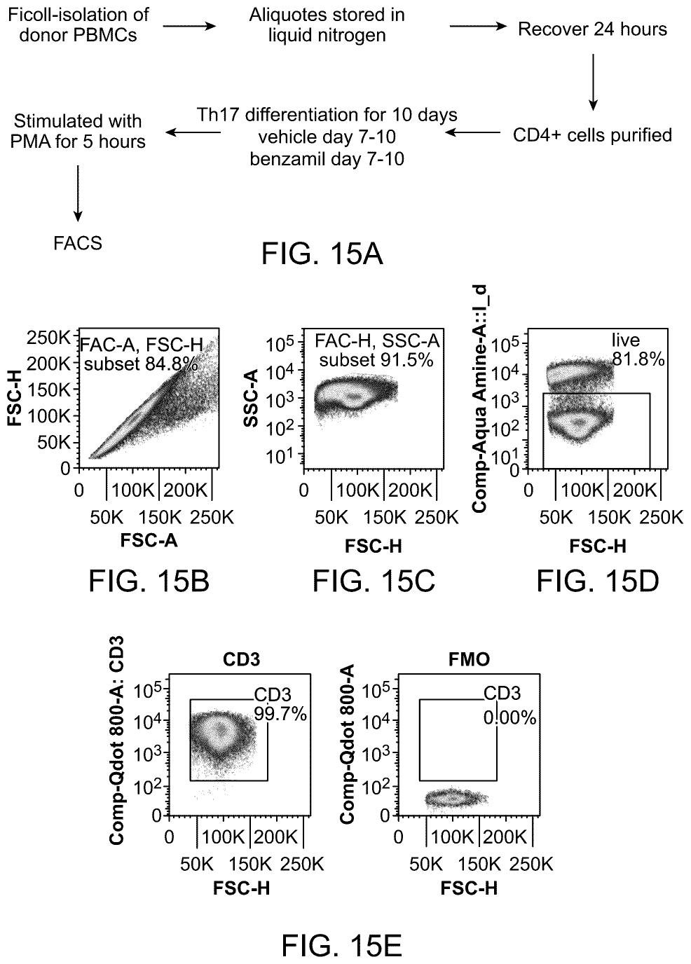

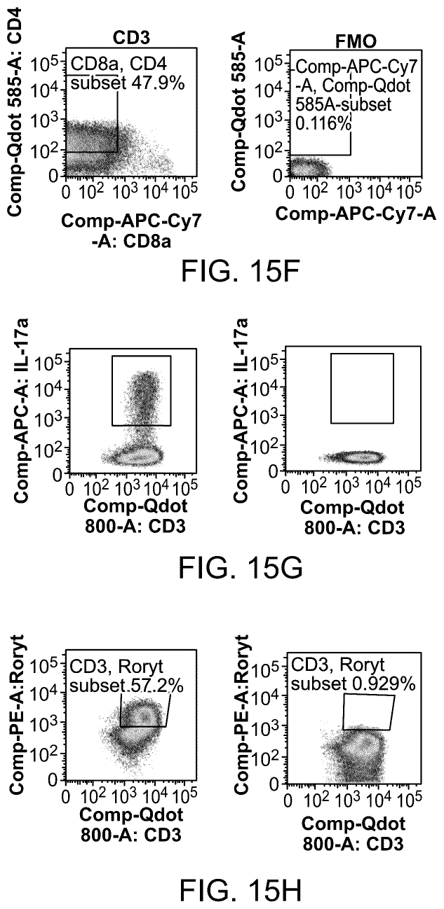

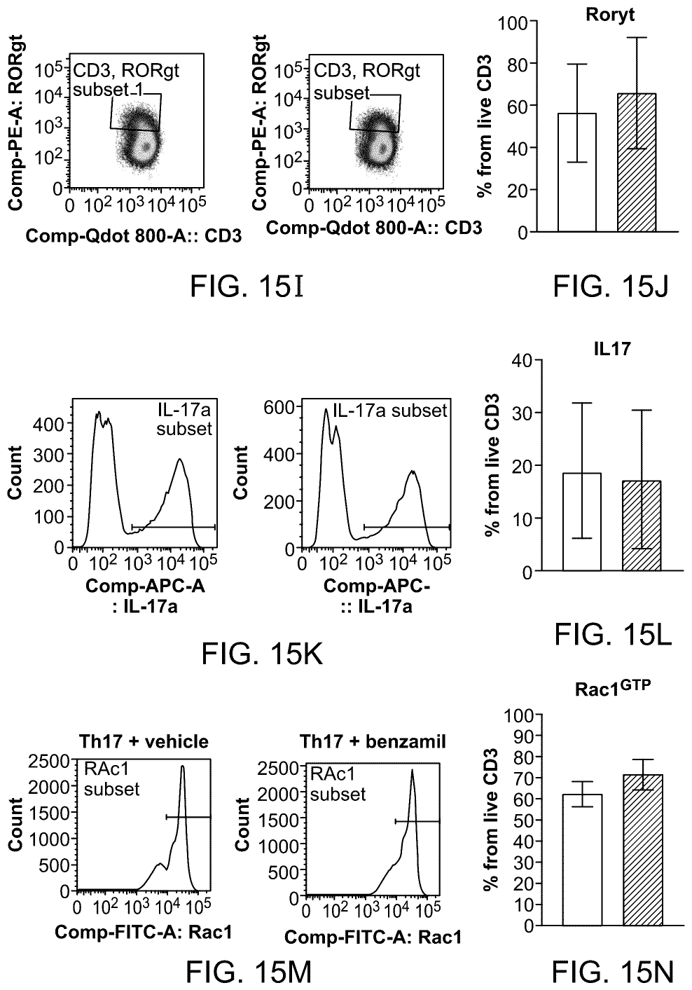

As an orthogonal approach, we used a monoclonal Rac1GTP antibody and measured Rac1GTP activity using confocal microscopy, and validated a significant reduction in Rac1GTP by benzamil, in Rac1-activated cells (FIG. 14C-D). Given that our in silico screen indicated a potential effect of benzamil on T-cell activation, we sought to test whether benzamil would affect the activation of Th17-differentiated naïve primary human T-cells using phorbol myristate acetate (PMA) (FIG. 15A-H). Unexpectedly, we did not detect a difference in the Th17 transcription factor Ror^{γ}t (FIG. 15I-J), IL17 production (FIG. 15K-L), or Rac1 activation (FIG. 15M-N), central for Th17 differentiation in T-cells, by benzamil.

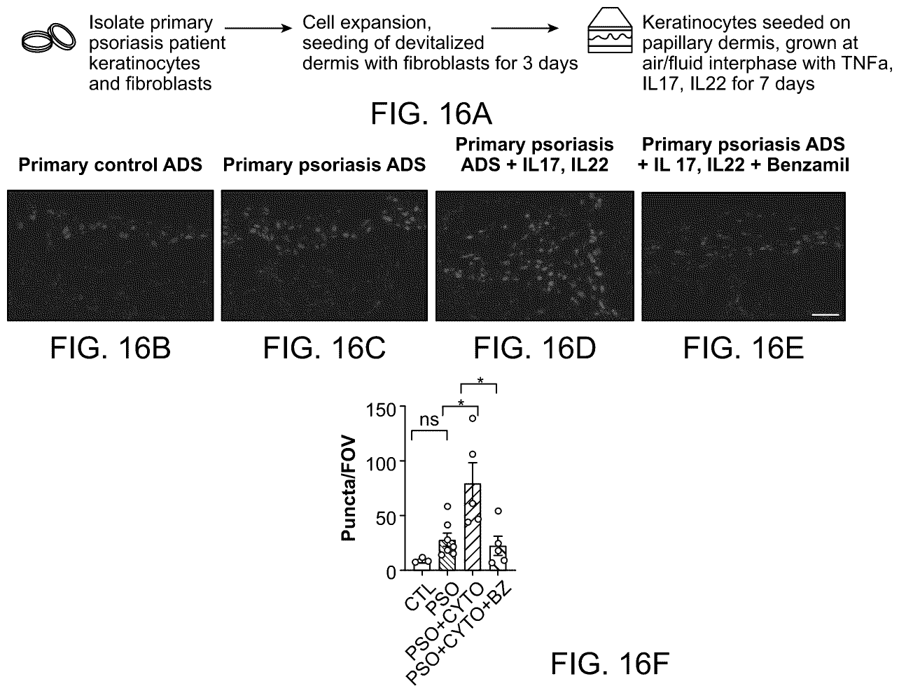

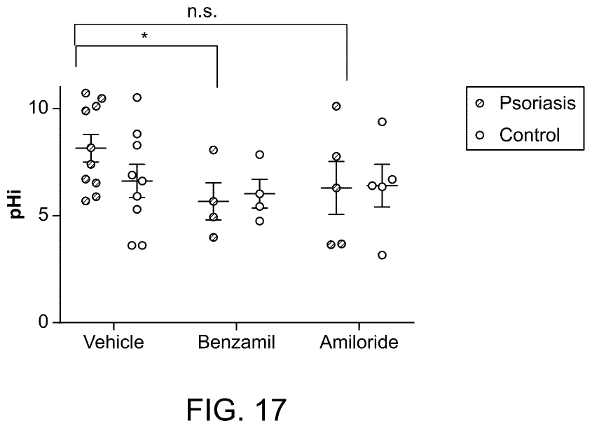

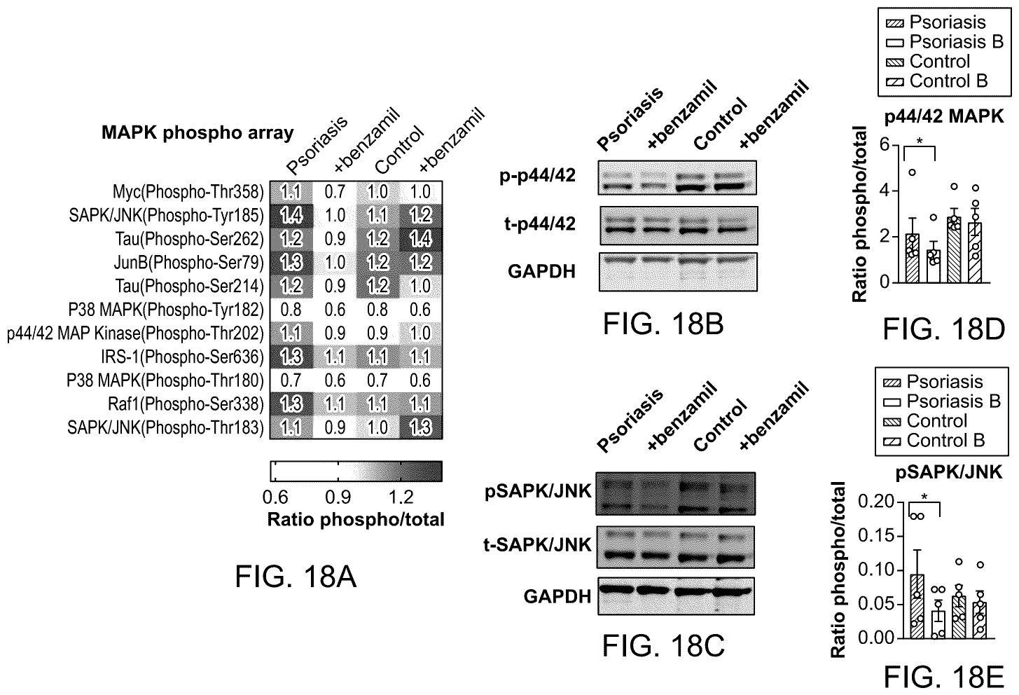

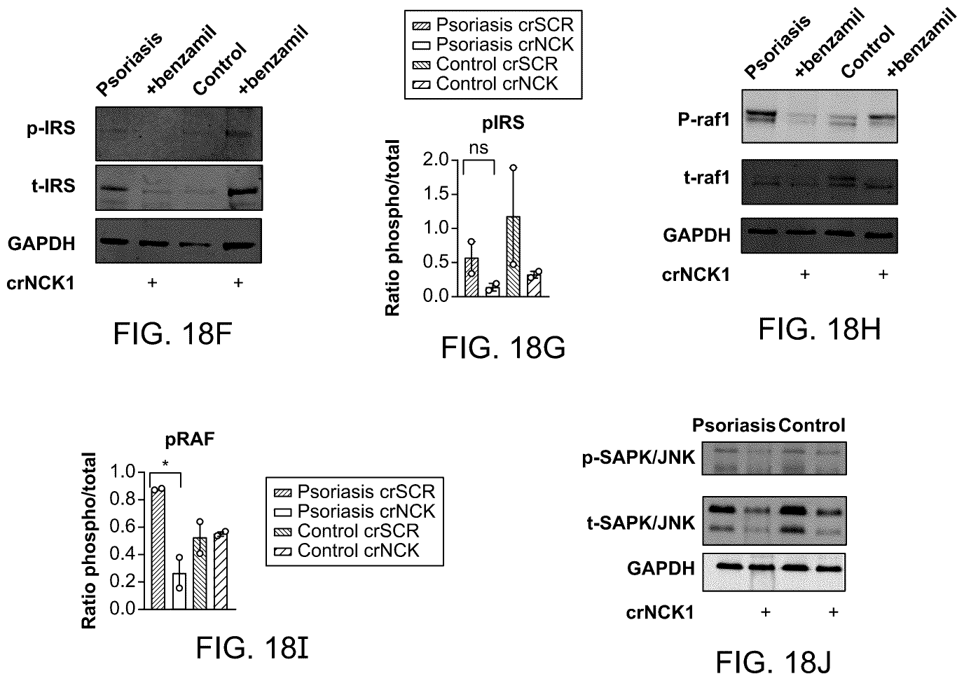

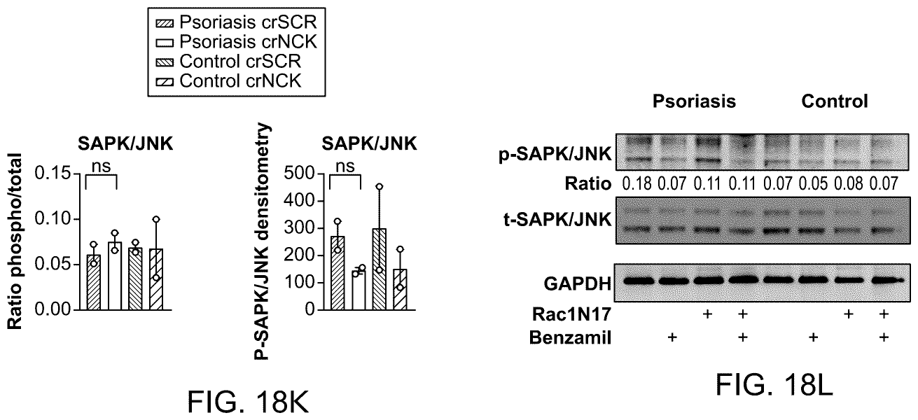

Taken together, our results suggest benzamil works primarily by inhibiting keratinocyte signaling in psoriasis. It has been shown that antagonism of the NHE channel can lead to inhibition of small GTPases via intracellular pH reduction, and that NHE and ENAC channels each regulate skin acidification in mouse models. Therefore, we tested whether benzamil reduced primary keratinocyte intracellular pH, and whether reduced intracellular pH could inhibit Rac1GTP. We found that benzamil did indeed reduce intracellular pH in primary keratinocytes overexpressing Rac1 (FIG. 4F, FIG. 16A), and that this was also achieved by the selective NHE inhibitor zoniporide, but not by the less potent ENAC/NHE inhibitor amiloride or another ENAC inhibitor, triamterene. Further, zoniporide recapitulated benzamil's significant capacity to reduce proliferation in primary keratinocytes, which was not achieved by amiloride or triamterene as a 10 μM concentration (FIG. 4G). To further validate that benzamil could inhibit Rac1 through altering intracellular pH and that this was associated with reduced Rac1 activation and altered intracellular localization, we assayed Rac1GTP activity by confocal microscopy in Rac1V12 keratinocytes and found a marked reduction in Rac1GTP by lowering intracellular pH, similar to the extent of 10 μM Benzamil (FIG. 16B-D). We found that loss of the sodium/hydrogen exchangers (ENAC, NHE1), but not calcium exchangers (NCX1) reduced Rac1GTP in psoriatic keratinocytes (FIG. 16E-F). Thus, these results establish that benzamil acts on Rac1-related signaling pathways in keratinocytes, at least partially through alteration of intracellular pH by sodium channel inhibition. To confirm a specific effect of benzamil on keratinocytes in the presence of immune-derived stimuli but absence of immunocytes, we generated organotypic 3D skin equivalents of psoriatic or control keratinocytes and fibroblasts on acellular dermal support (ADS) (FIG. 18A), which we previously demonstrated correlates well with xenografted psoriatic skin. We found that IL17, TNFα and IL22 stimulation of ADS grown at the air-fluid interphase led to epidermal hyperplasia, thus making for a suitable in vitro screening-model. Cytokine stimulation of control ADS led to a modest but insignificant increase in proliferation (Ki67+ cells) and epidermal thickness, whereas stimulation of psoriasis ADS resulted in a robust increase in proliferation that was abrogated by benzamil (FIG. 41-J). We also confirmed that increased phosphorylated STAT3 in psoriasis ADS correlated with cytokine stimulation, and was reduced with benzamil treatment. We were not able to correlate NFKB activation (through degradation of IKBa and nuclear translocation of P65) with cytokine stimulation or benzamil inhibition in psoriatic keratinocytes in vitro indicating that this mechanism involves stimulation by additional or other cytokines/chemokines than IL17, TNFα and IL22, or that the differential effect noted in vivo in Rac1V12 skin is through non-keratinocyte signaling in the epithelial-immune microenvironment.

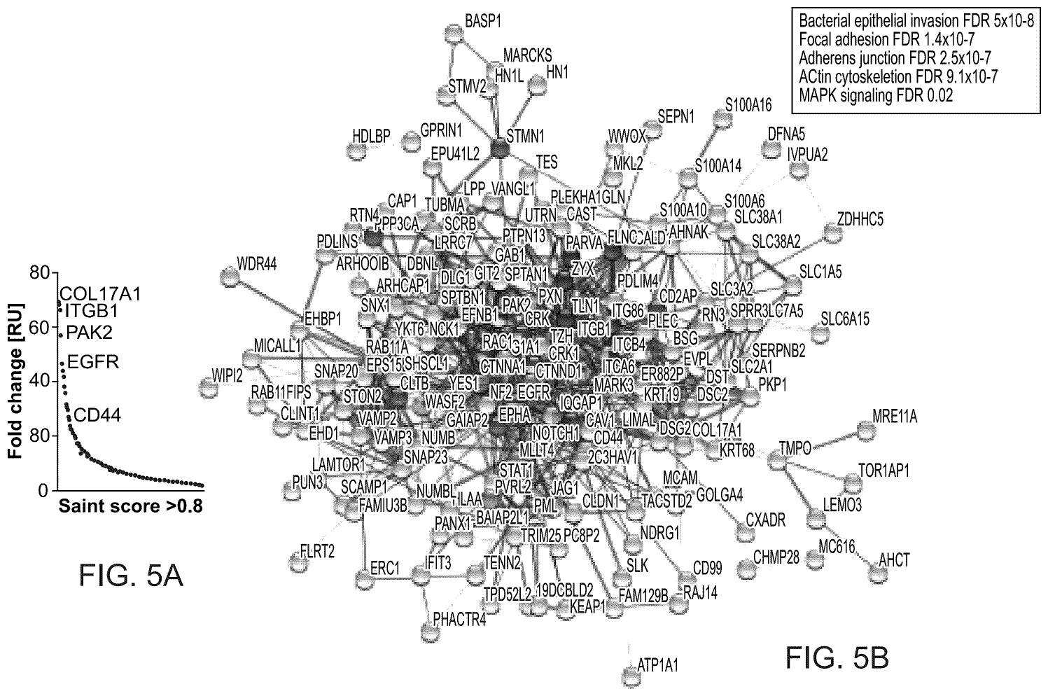

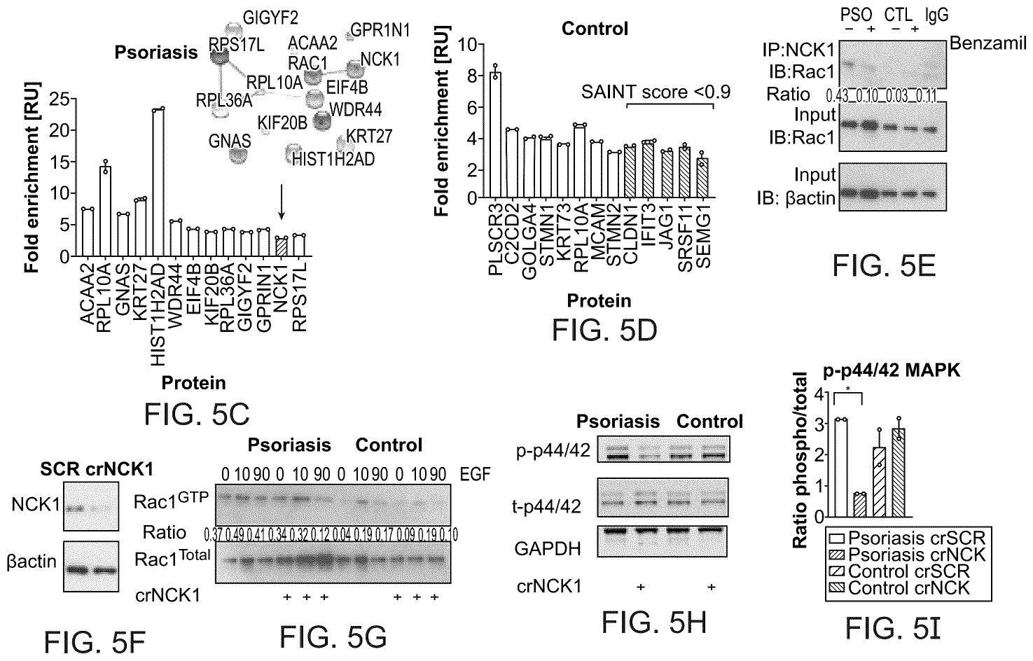

Abnormal Rac1-NCK interactions underlie Rac1 activation in psoriatic primary cells To understand how benzamil correlated with aberrant psoriatic epidermal Rac1 activity, we performed an assay compatible with the specific requirements of spatio-temporal activation and coordination of subsequent protein-protein interactions of small GTPases. We used BiolD, a vicinal proteomic method, to identify Rac1 interacting proteins in the presence or absence of benzamil. N-terminal BirA-tagged Rac1, or HA-control tagged BirA was expressed in psoriatic primary keratinocytes and primary control keratinocytes. Interacting proteins were identified as those scoring higher than 0.9 using the Significance Analysis of INTeractome (SAINT) algorithm. We validated our approach by identifying known interactors of Rac1. Indeed, known Rac1-interacting proteins such as COL17A1, ITGB1, PAK2, EGFR and CD44 were significantly enriched (FIG. 5A). We annotated the Rac1 protein interactome using stringdb and KEGG pathway analysis to further validate detection known Rac1-signaling pathways including focal adhesion, actin cytoskeleton, adherens junction and MAPK signaling (FIG. 5B). Next we compared the Rac1 interactome in primary psoriatic or control cells to benzamil treatment. Based on this analysis, benzamil treatment affected interaction of 13 proteins with Rac1 in primary psoriatic cells while affecting 8 proteins in control keratinocytes. Spectral counts from LC-MS/MS of biotin-tagged proteins differential between benzamil treated and untreated cells uncovered 13 proteins with differential proximity to Rac1 after benzamil treatment in primary psoriasis cells (SAINT score >0.9),whereas 8 had differentially proximity to Rac1 in normal control cells (SAINT score >0.9) (FIG. 5C-D). These enriched interactors with Rac1 in psoriasis patient cells were also annotated using STRING. NCK1 was predicted to directly interact with Rac1, and has been linked as an essential part of a multimeric complex that modulates Rac1 signaling (FIG. 5C), but not in primary control cells (FIG. 5D). We validated this interaction through co-immunoprecipitation of NCK1 and Rac1 in primary psoriatic keratinocyte lysates. Psoriatic or control cells were incubated with vehicle or benzamil for 24 hours, and NCK-Rac1 co-immunoprecipitated in psoriatic keratinocytes, but this interaction was diminished in benzamil treated cells, producing interaction levels seen normal control keratinocytes (FIG. 5E).

Subsequently, to confirm that NCK1-Rac1 direct interaction was required for excessive Rac1 activation in psoriatic epidermis, we used CRISPR-cas9 (crNCK1) to knockout NCK1 in primary psoriasis and control keratinocytes, and achieved a near but not complete knockout (FIG. 5F). Through Rac1GTP pulldowns via PAK pulldown beads, we demonstrated that loss of NCK1 reduced excess Rac1GTP after 90 minutes in primary psoriasis patient cells (FIG. 5G). . We next investigated whether benzamil could perturb MAPK signaling, a pathway that was identified through our bioinformatics screen.

Using MAP kinase phospho-arrays we compared the effect of benzamil on both psoriatic and control keratinocytes (FIG. 18A). We identified several phosphorylation-sites differentially altered by benzamil between patient and control cells, such asp-Tyr185 SAPK/JNK, p-Thr202 p44/42 as well as several that were increasingly phosphorylated in psoriatic cells, including p-Tyr185 SAPK/JNK, p-Thr202 p44/42, p-ser636 IRS and p-ser338 Raf1. We validated a differential effect of both p-p44/42 (FIG. 18B, D) and p-SAPK/JNK (FIG. 18C, E). We compared p-p44/42 levels in crNCK1 psoriasis and control keratinocytes and detected that crNCK1 patient cells had a significant reduction in p-p44/42 (FIG. 5H-1). We assessed two kinases upstream of p-p44/42 (Raf1) or SAPK/JNK (IRS) for dependency of NCK1. We found that p-RAF1 was significantly lower in crNCK1 psoriasis keratinocytes, whereas there was a (non-significant) trend of reduced p-IRS (FIG. 18F-I). Finally, we noted a (non-significant) trend of decreased phosphorylated and total SAPK/JNK (FIG. 18J-K). To verify that phosphorylated SAPK/JNK was Rac1 dependent, we overexpressed a dominant-negative mutant of Rac1 (FIG. 18L) (N17) in patient and control cells, which rendered the cells irresponsive to benzamil-driven downregulation of p-SAPK/JNK. Altogether, our findings establish that benzamil's action on Rac1 involves disassociation of Rac1 and NCK1, leading to significantly reduced Raf1-ERK and possibly IRS-SAPK/JNK signaling downstream of Rac1-NCK1.

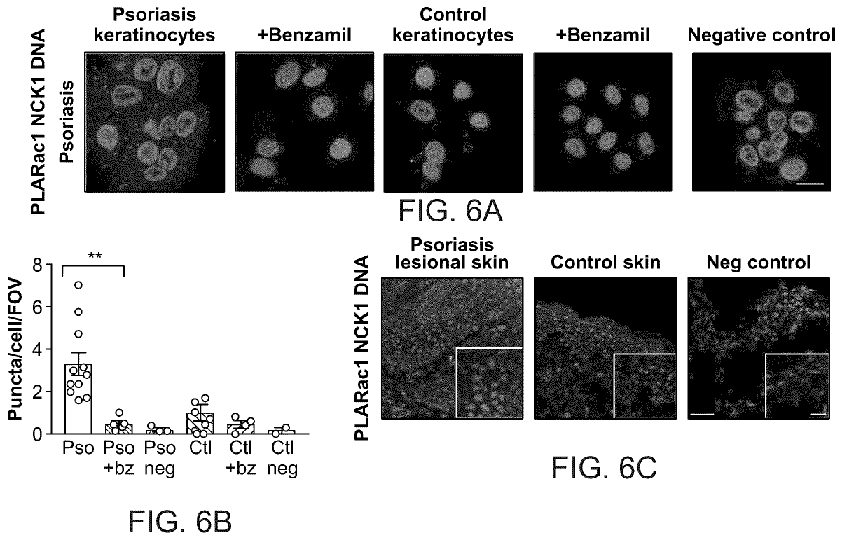

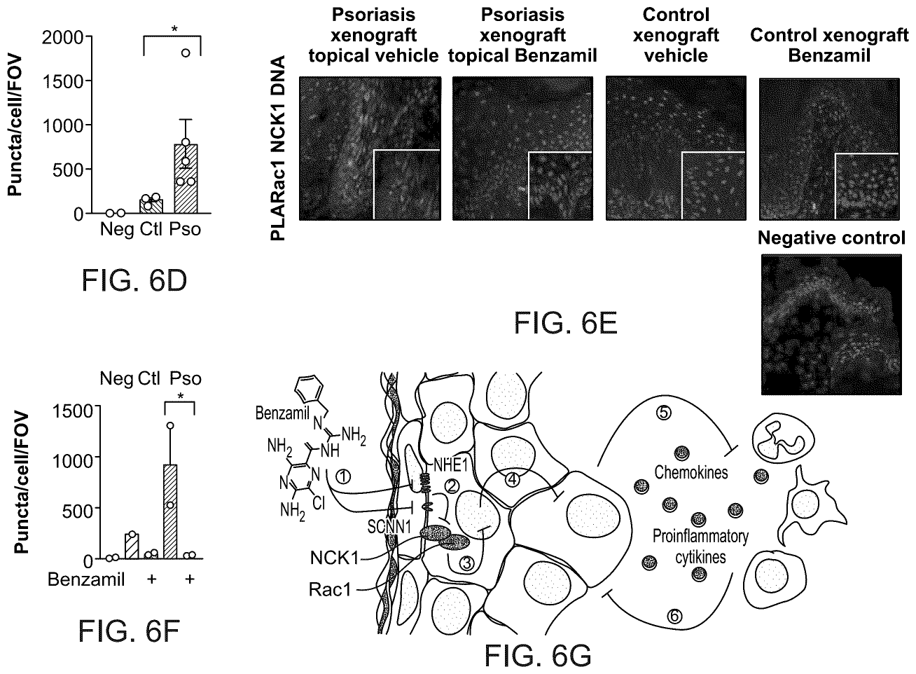

Topical benzamil rescues pathologic NCK-Rac1 interactions in primary reconstructed human psoriasis skin in vivo. To orthogonally validate whether benzamil perturbed Rac1-NCK1 in psoriatic cells, we also used proximity ligation assay (PLA) of NCK1-Rac1 in psoriatic and control keratinocytes treated with benzamil. Using this method, we found that benzamil significantly reduced NCK1-Rac1 proximity in psoriatic cells, congruent with our BiolD and co-immunoprecipitation results (FIG. 6A-B). We then evaluated Rac1 and NCK1 expression in psoriatic lesional and control skin by immunofluorescence using confocal microscopy, and detected substantial overlapping expression in psoriatic lesional skin. To determine whether these proteins were in fact in close proximity in vivo, we performed PLA of psoriatic and control skin and found a significantly increased proximity of NCK1 and Rac1 in psoriatic lesional skin compared to control skin. (FIG. 6C-D). Finally, we therefore tested whether benzamil inhibited Rac1-NCK1 interactions in vivo by treating reconstructed psoriatic and control skin xenografts. We show that benzamil strongly reduced Rac1-NCK1 PLA-proximity (FIG. 6E-F). Altogether, our results establish that benzamil inhibits Rac1 in psoriatic cells through preventing pathologic interactions between Rac1 and NCK1, likely by targeting sodium/hydrogen exchangers.

Tremendous progress has been made in development of recombinant immune targeting biologics. However high cumulative costs of repeat administration and the long term systemic immunosuppressive risks of these agents can limit their use in some patients. This is especially relevant in psoriasis, which typically is a lifelong disorder following its initial presentation and thus can require continual therapy for many years. To address this, we identified novel agents through computational repositioning, and demonstrated as an example that benzamil is a novel, immune-sparing, and effective treatment when delivered as a topical or systemic therapy in a validated mouse model of psoriasis and in primary reconstructed human psoriatic skin in vivo. We recently identified epidermal Rac1 activation as a central player in the abnormal interactions between the epidermis and the immune system and as a common feature of human psoriatic skin. Epidermal specific Rac1 activation promoted abnormal IL17 and IL23 mediated immune activity, and induced a cutaneous, arthritic and cardiovascular-metabolic phenotype in mice closely resembling human psoriasis. Conversely, inhibition of Rac1 rescued psoriatic hyperplasia and inflammation in primary reconstructed primary human patient skin in vivo.

Our prior results suggest a possible intrinsic predisposition for Rac1 hyperactivation in psoriatic patient cells. Here, we demonstrate at least one clue to how this could be mediated, as we reveal a mechanism whereby Rac1 binds to the adaptor NCK1 differentially in patient versus control cells. That benzamil has a stronger Rac1 inhibitory effect in patient cells aligns well with the fact that it seems to normalize Rac1 activation through inhibition of Rac1-NCK1 interactions, which are increased in psoriatic epidermis. Benzamil's selective ability to inhibit psoriatic but not normal epidermal proliferation may similarly derive from the marked enrichment of Rac1-NCK1 interactions in psoriatic versus normal epidermis.

Benzamil inhibits excess chemotactic signals in inflamed skin, reducing expression of CXCL1, CXCL2, CCL2, CCL7 as well as TNF□. CXCL1 has recently been implicated in keratinocyte-driven NF□B signaling in conjunction with TNF□, is IL22-inducible and exhibits potent chemotactic properties. IL22 is abundantly produced by Th17 cells in human psoriasis and in the Rac1V12 psoriasis mouse model. Further, IL22 activates Rac1 in keratinocytes, and stimulates keratinocyte proliferation. Therefore, Rac1 inhibition likely prevents IL22-mediated pro-proliferative and chemotactic signals such as CXCL1 production. Several of these cytokines are intrinsic for innate immune signaling, such as CXCL2 recruiting innate immune cells such as neutrophils and monocytes that are needed for skin inflammation. Interestingly, we failed to detect a significant difference in Th17 differentiation from CD4+ human primary cells. However, benzamil dependent cytokines such as CCL2 have been shown to promote recruitment of IL-17A-producing γδT cells. This provides a compelling example how benzamil, through Rac1-inhibition, perturbs the pathologic epidermal-immune feedback loop in psoriatic skin.

These studies highlight the key role of the epidermis in driving abnormal psoriatic immune activity and how targeting epidermal Rac1 can be an effective and safe alternative or complement to systemic immunosuppressive therapies currently in use. Our results link sodium channel inhibition by benzamil, to the perturbed interactions between NCK1 and Rac1. Our results present a unifying model where benzamil regulates Rac1 activation through its association with NCK1 by a combined effect on altering sodium/hydrogen flux and thereby intracellular pH.

We compared the robust effect of benzamil to its older generation ENAC inhibitor amiloride, which is an FDA approved, less potent ENAC inhibitor. We failed to demonstrate any effect of systemic amiloride on psoriasiform hyperplasia in the Rac1V12 mouse model of psoriasis, and amiloride lacked an inverse signature to psoriasis in the drug repositioning algorithm. Our combined results suggests that it is the unique combined target profile of benzamil that underlies its therapeutic potential for psoriasis. This combinatory signature was found through a validated drug repositioning approach. Interestingly, the NHE inhibitor zoniporide resembled the effects of benzamil.

### Material & Methods

Psoriasis Gene Expression Data. Psoriasis gene expression data were obtained from the national center for biotechnology (NCBI) gene expression omnibus (GEO). We used five gene expression datasets (GSE6710, GSE11903, GSE13355, GSE14905, GSE30999), which were run on Affymetrix GeneChip HG-U133A, HG-U133 2.0 and HG-U133plus2 platforms. Each dataset consisted of samples obtained from lesional skin (LS) and non-lesional skin (NLS) of patients with psoriasis (Table 2). A total of 200 LS and 196 NLS microarrays were used for analysis.

Meta-Analysis of Psoriasis Gene Expression Data A rank aggregation algorithm was used to meta-analyze psoriasis gene expression data using the R statistical software and the GeneExpressionSignature package from Bioconductor. In brief, the method hierarchically merges ranked lists to generate a single ‘prototype’ ranked list (PRL), and depends on three simple tenants: a) a measure of list similarity (the Spearman's Footrule), b) a method of merging lists (the Borda merging method), c) a method of hierarchical clustering (the Kruskal algorithm). The outcome of this step, was a consensus signature for lesional psoriatic skin compared to non-lesional skin.

Drug Repositioning using the MANTRA tool. The Java-based publicly-available online tool, Mode of Action using Network Analysis (MANTRA) version 1.0, was used to computationally predict drug candidates that can be repurposed in psoriasis. In its original description, the MANTRA tool was used to generate a drug-drug network based on the similarity of the transcriptomic profiles of 1309 drugs used in the Connectivity Map. We adapted the use of this tool for GEO derived disease gene expression data. For this, the psoriatic PRL of NLS vs LS was generated as described above and analyzed using the MANTRA online tool. A total enrichment-score (TES) distance was calculated using the MANTRA tool for each drug-PRL vs disease-PRL pair. The TES represents statistical measure of similarity between two gene signatures, ranging between 0 and 2, where the smaller TES values indicate increased similarity between the drug and disease signatures (0 represents two identical gene lists). A TES cutoff of 0.8 was chosen as a threshold of significance corresponding to the 5% quantile of the empirical probability distribution function of the drug-drug distances as previously described.

Characterization of functionally deregulated pathways. To identify functional pathways that are important in psoriatic lesional skin as compared to non-lesional skin, enrichment of gene ontology (GO) gene sets within the PRL of psoriasis was measured using gene set enrichment analysis (GSEA). GSEA enriched gene-sets with a FDR s 0.001 were then visualized using the Enrichment Map tool plugin in Cytoscape. An overlap coefficient of 0.60 was used to connect gene sets enriched in the psoriasis PRL. To characterize the distribution of genes modulated by benzamil, a gene set was generated for benzamil by taking the 100 most up-regulated and 100 most down-regulated genes from the PRL of benzamil. Using the post-analysis feature from the Enrichment Map tool, statistically significant connections, using a hypergeometric test with a cutoff of 0.1, of the benzamil gene-set to GO gene-sets were then visualized. Ontology terms were also enriched for the benzamil-psoriasis dataset using BIOCARTA metabolic pathways using enrichR. Relating protein pharmacology by ligand chemistry Numerous compounds in the Connectivity Map are identified by their vendor names, and are commercially unavailable.

To identify protein pharmacology based on ligand topology, we used a method previously described. In brief, ChemBank was queried used vendor compound names to identify the simplified molecular-input line-entry system (SMILES) chemical structure. The SMILES code was then queried to identify top ranking target ligands.

Genotype-tissue expression analysis The Genotype-Tissue Expression (GTEx) Project was supported by the Common Fund of the Office of the Director of the National Institutes of Health, and by NCI, NHGRI, NHLBI, NIDA, NIMH, and NINDS. The data used for the genotype-tissue expression analyses described in this manuscript were obtained from the GTEx Portal on Feb. 15, 2020.

Transgenic mice and mice for xenografting Rac1V12-transgenic mice were generated as previously described. For xenografting, NOD/SCID mice were used (NOD.CB17-PrkdcSCID/J mice; stock 001303; The Jackson Laboratory).

Topical formulation and administration. A rudimentary preparation of benzamil was tested using benzamil (6 mg/ml, benzamil, Sigma) in 100% ethanol. This solution was then topically applied through submerged and air-drying tails of mice under anesthesia, once daily for 15 days. Similarly, benzamil was suspended in water (2 mg/ml), then mixed into Vaseline, vortexed and centrifuged until a clear solution was obtained. A final formulation of benzamil is described in supplementary material and methods. For intraperitoneal injections, benzamil was dissolved at 2 mg/ml in water, and 0.7, 1.4 or 2.1 mg/kg was injected IP daily. For osmotic pump injections, Alzet osmotic pumps were used, (Alzet minipump 1002). Pumps were filled with 100 ul of benzamil in ethanol under sterile conditions, at a final dose of 2 mg/kg/day, and subcutaneous, inter-scapular implantations were performed under anesthesia. Pumps were replaced once for a total of 2×14 days treatment period.

Intracellular pH assays 25,000 were seeded per well of 96-well plate and allowed cells to settle and recover for 24 hrs. Small molecules or 0.1% DMSO vehicle control was added to culture media for 24 hours (n=6-8). Cells were washed once with 1× Live Cell Imaging Solution (LCIS) (ThermoFisher A14291DJ), and then incubated with pHrodo red (ThermoFisher P35372), PowerLoad (ThermoFisher P35372), in LCIS with added 1:5000 Hoechst (Life Technologies) for 30 min at 37 C 5% CO2 with corresponding small molecule added at same concentration used for 24 hour incubation.

cDNA, siRNA and sgRNA constructs and vector information. V12 Rac1, V14 RhoA, dominant negative N17 Rac1 or LacZ control constructs were generated and cloned as previously described. Human V12 Rac1, N17Rac1 and LacZ constructs were a kind gift of Dr John Collard, Nethelands Cancer Institute, Amsterdam, The Netherlands. For CRISPR/Cas9 knockout human keratinocytes were transduced with lentivirus generated using lentiCRISPRv2, as previously shown effective in keratinocytes to introduce sgRNAs targeting either SCNN1α (GGCAGCCTCACTCGGGTTCC), NCX1 (AAGGGGTGATTTTGCCCATT), NCK1 (AGGAAGATCAATGGTATGGT), NHE1 (CCAATCGAGCGTTCTCGTGG) or non-targeting control (CAGACCGAACCCTAAAGA).

Cell culture Neonatal foreskin, adult human control or adult human psoriatic non-lesional skin was incubated overnight at 4° C. in HBSS with 25 U/ml dispase. Only non-lesional skin was harvested for in vitro analysis and xenograft production. Epidermis-dermis was separated with forceps, epidermal sheets trypsinized for 15 min, neutralized with DMEM Mediatech Inc) containing 10% FBS, 1% antibiotic-antimycotic (30-004-CI, Mediatech Inc), centrifuged for 5 min at 1000 rpm, and re-suspended in a 50-50 mixture of supplemented (Human Keratinocytes Growth Supplement, S-001-5, Invitrogen) Medium 154 (M16 254-500, Invitrogen) and K-SFM (Defined Keratinocyte SFM, 10744-019, Invitrogen) and 1% antibiotic-antimycotic solution (0-004-CI, Mediatech Inc). Adult cells were at passage 3 prior to in vitro analysis or passage 2 for xenografting experiments. PBMCs were isolated from whole blood using Ficoll-Paque (GE Healthcare) according to manufacturer's recommendations. PBMCs were incubated in RPMI with 10% FBS for 24 h prior to injection, magnetic bead sorting or FACS analysis.

Cytokine stimulation. Primary human adult psoriatic or adult normal control keratinocytes were growth factor starved for 24 hours then stimulated using 50 ng/ml EGF (PHG0311, Life Technologies).

MTT assays. 10000 third-passage adult keratinocytes per well, or 25000 for IC50 curves, were seeded on a collagen-coated 96 well plate, and incubated with 50/50 (Medium 154 and Keratinocyte—SFM medium, Invitrogen with Human Keratinocytes Growth Supplement, S-001-5, Invitrogen, and Medium 154 supplement M-154-6 500, Invitrogen) with 1% antibiotic-antimycotic (30-004-CI, Mediatech Inc) for 48 h. 10 ul MTT reagent (MTT proliferation assay, ATCC, Manassas, US) was added for 4 h, 100 ul detergent was added to each well for 4 hours, and absorbance read at 570 nm (Spectramax M5, Molecular Devices, US), normalized to cell-free control absorbance.

Small molecule inhibition. All small molecules were diluted into concentrated stock solutions (all except YM-244769 were made into 50 mM stock solutions) in DMSO, and stored in aliquots at −20 C. Aliquots were thawed, then diluted to a 1000× stock concentration, then added to media so that final DMSO concentration is 0.1%. All in vitro studies entailing small molecule inhibition were accessed over 24 hour incubation period.

RNA extraction and RT-qPCR. Quantitative reverse transcription PCR (RT-qPCR) was performed using the Roche LightCycler 480 with Maxima SYBR Green Master Mix (Fermentas) or SYBR Select Master Mix (Invitrogen). Samples were run in triplicate and normalized to Rn18s RNA. RNA was extracted from cell lysates; or mouse skin using a Qiagen RNA plus miniprep kit. RNA concentration was determined with spectrophotometric analysis; purity analyzed with 260:280 absorbance ratios. One ug of RNA was reverse-transcribed using iSCRIPT cDNA synthesis kit (Bio-Rad) or High Capacity RNA-to-cDNA Kit (Invitrogen). For some experiments, RNA clean up using “GeneJET RNA Cleanup and Concentration Micro Kit” (Fisher K0841/FERK0841) was used, utilizing 765 ng of RNA per sample, then directly made cDNA using the High capacity RNA-to-cDNA kit (Invitrogen).

FACS analysis. CD3+ cells were isolated using CD3 microbeads (Miltenyi, 130-050-101) according to manufacturer's recommendations. Th17 differentiation was performed by stimulation with Recombinant Human TGF-beta 1 Protein (R&D systems, 240-B-002), Recombinant Human IL-23 Protein (1290-IL-010, R&D systems), Recombinant Human IL-6 (550071, BD Bioscience) together with Purified NA/LE Mouse Anti-Human IFN-γ Clone B27 (BD Biosciences, 554698) and Purified NA/LE Rat Anti-Human IL-4 Clone MP4-25D2 (BD Biosciences, 554481), Purified NA/LE Mouse Anti-Human CD28 Clone CD28.2 (RUO) (BD Biosciences, 555725), Anti-hamster IgG (for coating) (Fisher, AI-9100). Intracellular staining was performed using Foxp /Transcription Factor Staining Buffer Set (eBioscience, 00-5523-00) and BD Cytofix/Cytoperm™ Fixation/Permeabilization Solution Kit with BD GolgiStop (BD Biosciences, 554715). For stimulation Leukocyte Activation Cocktail, with BD GolgiPlug™ was included (BD Biosciences. 550583). Antibodies for FACS can be found in supplementary material and methods.

BiolD IDT block encoding Rac1 (NM_006908.4) was ordered and cloned in-frame of HA-BirA with a rigid linker of 15 amino acids (EAAAK)n=3 in between BirA and Rac1. For sample preparation for LC-MS/MS, streptavidin biotin magnetic particles were washed with 500 uL 50 mM ammonium bicarbonate three times. Beads were re-suspended in 200 uL 50 mM ammonium bicarbonate with the addition of DTT to a final concentration of 5 mM, incubated on a heat block at 50° C. for 5 min followed by end over end rocking for 30 minutes at RT. Alkylation was performed by the addition of propionamide to a final concentration of 10 mM and head over head shaking for 30 minutes at RT. 250 ng of trypsin/LysC (Promega) was added to each sample and digested overnight at RT in the end over end shaker followed by the addition of formic acid to 1%. Peptides were removed and washed with 50 uL 0.1% formic acid water. The acidified peptide pools were C18 STAGE tip purified (NEST group) using microspin columns and dried in a speed vac. For LC-MS/MS, peptide pools were reconstituted and injected onto a C18 reversed phase analytical column, 10 cm in length (New Objective). The UPLC was a Waters NanoAcquity, operated at 600 nL/min using a linear gradient from 4% mobile phase B to 35% B. Mobile phase A consisted of 0.1% formic acid, water, Mobile phase B was 0.1% formic acid, water. The mass spectrometer was an Orbitrap Elite set to acquire data in a data dependent fashion selecting and fragmenting the 15 most intense precursor ions in the ion-trap where the exclusion window was set at 60 see and multiple charge states of the same ion were allowed.

MAPK phosphoarray 5×10⁶ primary human keratinocytes from non-lesional psoriasis or control skin were incubated with vehicle or benzamil (10 μM) for 24 hours, lysed and processed using the Antibody Array Assay Kit (KASO2, Full Moon biosystems) onto MAPK phosphoarrays (KASO2, Full Moon biosystems) according to manufacturer's instructions. Cy®3-Streptavidin was used as secondary antibody and arrays were scanned on a GenePix 4200 Auto Loader.

Proximity Ligation assay. Duolink In Situ Orange PLA kits were used (Sigma, DU092102). Briefly, Methanol fixed samples were washed, blocked with blocking solution for 30 min, decanted, and emerged in primary antibody mixture overnight. Samples were washed in wash buffer, and incubated with PLA probes for 60 min at 37° C. Samples were washed for 2×5 min, and incubated in ligation mix for 30 min at 37° C. Samples were washed 2×2 min, and incubated in amplification mix for 100 min at 37° C. Samples were washed in wash buffer 2×10 min, followed by 0.01× wash buffer for 1 min, prior to being mounted with Duolink Mounting Medium with DAPI and imaged.

eBioscience/Affymetrix Magnetic bead Kits Luminex assays were performed in the Human Immune Monitoring Center at Stanford University. Mouse 38 plex kits were purchased from eBioscience/Affymetrix and used according to the manufacturer's recommendations with modifications as described in supplementary material and methods.

Human xenografts. Xenografts comprised of primary psoriasis patient or primary control cells were generated as previously described with minor modifications. Typically, keratinocytes were grown in supplemented 50/50 medium-154 and defined keratinocyte SFM as described, and fibroblasts in DMEM (Mediatech Inc), with 10% FBS. 0.5×10⁶ fibroblasts were centrifuged (2×20 min, 1000 rpm) onto reticular side of a 10×10 mm2 11 devitalized dermis (New York Firefighters Biobank, NY, US), and cultured in DMEM with 10% FBS for 3 days. Skin equivalents were transferred to an annular dermal support (ADS) tissue culture insert. 35 mm plastic inserts were prepared with an 8 mm2 square central orifice resting on anchored 3 mm glass beads inside a 60 mm plastic tissue culture dish. Reticular side of skin equivalent was covered in matrigel (BD Biosciences), dried for 5 minutes and flipped onto 35 mm insert, covering an 8 mm2 orifice, exposing papillary side of skin equivalent to the air-fluid interphase. 1×10⁶ adult, first passage, non-lesional primary psoriatic or adult control keratinocytes were seeded in 100 ul 50/50 M-154/defined keratinocyte SFM media into orifice on papillary side, settled for 10 minutes, and 5 ml of KGM pipetted into lower chamber comprising of a 60 mm tissue culture dish. Media was changed in lower chamber daily for 7 days. On day 8, skin equivalents containing psoriatic keratinocytes and autologous fibroblasts or control keratinocytes and autologous fibroblasts were grafted onto 8 week old NOD/SCID male mice (Jackson Labs), sutured and bandaged. Bandages were removed on day 7 and the grafts were exposed. At this point, blood samples had been obtained from each subject, and PBMCs were isolated using Ficoll-paque plus per manufacturer's recommendations (GE Healthcare). 150 μl of RPMI with PBMCs (1×10⁶) were injected intradermally into psoriasis (n=5), or control xenografts (n=5) upon suture removal. The following day, grafts were topically treated with 100 μl each of either formulated benzamil, or vehicle control daily for 15 days, when all grafts were harvested.

Organotypic 3D skin equivalents. Organotypic 3D skin equivalents were generated as previously described with minor modifications. Media was changed in lower chamber daily for 7 days and was for stimulated conditions supplemented with IL17, IL22 and TNFα, or IL17, IL22, TNFα and benzamil (10 μM). Samples were harvested, embedded in OCT, and snap-frozen.

Statistical analysis. Microrarray data was analyzed using the affy package from Bioconductor on R statistical software package. Other statistical analysis was performed using Graphpad Prism 7.03, and statistical tests (unpaired t-tests with Welch correction, paired t-test, Mann-Whitney unpaired test, and linear regression analysis with goodness of fit) and sample sizes are outlined in each figure legend. Components of signature for goodness of fit by linear regression analysis were extrapolated from the benzamil rank aggregation algorithm All error bars denotes standard error of the mean. For IC50 calculations, MTT data absorbance values were calculated for percent change from the 0.1% DMSO vehicle condition. A non-linear fitted curve [log (inhibitor) vs response (three parameters)] in GraphPad Prism 7 was used to determine IC50 values. For mouse and xenograft experiments, ad hoc power calculations were based on prior phenotypic characterization of these models. Transgenic mice were randomized from same litters when possible, or based on similar phenotypic disease severity, and all experiments were run in triplicates or more. Sample size of xenografts mice studies were based on patient sample availability and a predicted graft take rate of 80%. For all comparisons, p values of less than 0.05 were considered statistically significant. All tests were two-tailed unless specified in figure legends.

Study approval. Mouse studies were approved by and conducted in accordance with Stanford University IACUC guidelines (assurance no. A3213-01, protocol 10364). Human studies were conducted according to Declaration of Helsinki principles, in agreement with approved human subject protocols of the IRB of Stanford University School of Medicine (protocol 30586). Informed consent was obtained from all human subjects.

Data and materials availability. All gene expression datasets are publically available and can be found in the NCBI GEO (GSE6710, GSE11903, GSE13355, GSE14905, and GSE30999). Rac1^{V12} mice were created in the Marinkovich lab.

Analysis of gene expression microarray data. Gene expression microarray data were analyzed using the affy package from Bioconductor on R statistical software package. First, quality assessment and control were performed and low-quality samples were removed from further analysis. Background correction and rank normalization was applied to the disease expression data using Robust Multiarray Average (RMA) package. For each dataset, the gene expression fold change between NLS and LS was calculated to generate a list of up-regulated and down-regulated genes in NLS compared to LS. We standardized ranked lists from all datasets to the Affy HG-U133A platform by filtering and/or appending the HG-U133A probesets to non-HG-U133A platforms. The gene lists, each consisting of 22,283 genes conforming to the Affy HG-U133A platform, were then sorted according to gene expression fold-change, from the most down-regulated in LS to most up-regulated in LS, to produce a ranked list of ‘normality’ (L) for each dataset.

LC-MS/MS data analysis. MS/MS data were analyzed using both Preview and Byonic v1.4 (ProteinMetrics). All data were first analyzed in Preview to provide recalibration criteria and then reformatted to .MGF before full analysis with Byonic. Analyses used Uniprot canonical .fasta files for human, concentrated with common contaminant proteins. Data were searched at 10 ppm mass tolerances for precursors, with 0.4 Da fragment mass tolerances assuming up to two missed cleavages and allowing for N-ragged tryptic digestion. These data were validated at a 1% false discovery rate using typical reverse-decoy techniques. The resulting identified peptide spectral matches and assigned proteins were then exported for further analysis using custom tools developed in MatLab (MathWorks) to provide visualization and statistical characterization. SAINT scores were generated using the online CRAPome tool with spectral counts from experimental samples and controls.

Processing of eBioscience/Affymetrix Magnetic bead Kits. Beads were added to a 96 well plate and washed in a Biotek ELx405 washer. Samples were added to the plate containing the mixed antibody-linked beads and incubated at RT for 1 h followed by overnight incubation at 4° C. with shaking. Cold and room temperature incubation steps were performed on an orbital shaker at 500-600 rpm. Following the overnight incubation plates were washed in a Biotek ELx405 washer and then biotinylated detection antibody added for 75 min at RT with shaking. Plate was washed as above and streptavidin-PE was added. After incubation for 30 min at RT wash was performed as above and reading buffer was added to the wells. Each sample was measured in duplicate. Plates were read using a Luminex 200 instrument with a lower bound of 50 beads per sample per cytokine. Custom assay Control beads by Radix Biosolutions were added to all wells. All samples were run in technical duplicates and fold changes were calculated between Rac1_{V12} vehicle-, Rac1_{V12} benzamil-treated and WT control mice.

In vitro luminex assay: Primary psoriasis patient keratinocytes at passage 3 were cultured to similar sub-confluent densities of about 45% in 12 well plates in 50:50 media, then treated with 10 uM benzamil hydrochloride hydrate (Sigma B2417) (n=3) or 0.1% DMSO vehicle (n=3). Media with 0.1% DMSO was added to wells containing no cells (n=3) and similarly incubated at 37 C 5% CO2. After 24 hour incubation, media supernatant was collected off the adherent keratinocytes, centrifuged at 10,000G for 10 min at 4 C, then snap frozen in liquid nitrogen and stored at −80 C. Samples were submitted to Stanford's Human Immune Monitoring Center to perform Human 62-plex Luminex assay.

### Gene Transfer

Lentiviral gene transfer. 293T cells were transfected with 8 ug of lentiviral expression construct, 6 ug of pCMVD8.91, and 2 ug of pUCMD.G. Transfections were done in 10-cm plates using Lipofectamine 2000 (Life Technologies). Viral supernatant was collected 72 h after transfection and concentrated using a Lenti-X concentrator (Clontech). Cells were transduced with lentivirus in presence of polybrene (5 μg/ml) overnight. After recovery, cells were selected with puromycin for 24 h.

Retroviral gene transfer. Phoenix cells were transfected with V12Rac1, V14 RhoA, N17 Rac1 or LacZ in 10-cm plates using Lipofectamine 2000 (Life Technologies). Cells were grown to 80% confluence, transferred to 32° C. incubation, and viral supernatant was collected after 24, 48 and 72 h. Cell cultures were incubated with polybrene for 10 min at 37° C., (5 ug/mL), media replaced by viral media with polybrene (5 ug/mL), centrifuged 1 hour at 1000 rpm, followed by incubation for 4 h at 37° C., prior to media change. After recovery, cells were selected with blasticidin for 48 h.

siRNA transduction and sequences. 1×10⁶ cells were electroporated with 1 nmol siRNA using Amaxa Human Keratinocyte Nucleofector Kit (Lonza VPD-1002), with control (D-001810-10-05, Thermo Scientific). SLC8A1 (L-007620-00-0005, Dharmacon) or SCNN1α (L-006504-00-0005, Dharmacon) siRNA.

Small molecule inhibitors. 10 uM benzamil hydrochloride hydrate (Sigma B2417), 10 uM amiloride hydrochloride (Tocris 890) 10 uM zoniporide dihydrochloride (Tocis 2727), 10 nM YM-244769 (Tocris 4544), 10 uM SEA 0400 2-[4-[(2,5-944 difluorophenyl)methoxy]phenoxy]-5-ethoxyaniline) (Tocris 6164), 10 uM triamterene (Sigma-Aldrich T4143).

Cytokine stimulation. Primary human adult psoriatic or adult normal control keratinocytes were stimulated with 50 ng/mL EGF (Life Technologies) or a cocktail of 25 ng/mL of IL-22 (Novus/Fisher), 100 ng/mL TNF(GIBCO/Invitrogen), 100 ng/mL IL17 (Thermo).

Intracellular pH assays and standard curves. Cells were also washed 3 times with 1×LCIS with the last wash of 100 uL kept on cells. For generation of pH standard curve, additional wells (n=6-8) were treated as described with an addition of 0.1% DMSO vehicle for 24 hours. After 3 1×LCIS washes, cells were incubated with 100 uL of Intracellular pH clamping buffers (pH 7.5, 6.5, 5.5, and 4.5) (ThermoFisher P35379) for 5 min at 37 C in 5% CO2. The 96-well plate was then read on a fluorescence microplate reader (Spectramax M5, Molecular Devices) with excitation/emission of 590/585 and 361/497. Intracellular pH standard curve was generated by either directly plotting pHrodo red ex/em or plotting pHrodo red ex/em divided by Hoechst ex/em.

Topical formulation of benzamil. A labrasol (88%) based formulation of benzamil was generated containing benzamil (1%, w/w), span-20 (1%) and cetyltrimethylammonium bromide (CTAB) (10%). Constituents were dissolved in ethyl alcohol, mixed well using vortex and sonication at 37° C. until a clear solution was obtained. Ethyl alcohol was evaporated in a Speedvac. The residue was suspended in Labrasol and homogenized to obtain the final formulation.

For a final formulation of benzamil (for use on human reconstructed skin), 52 ml water, 5.2 ml glycerin, 9.7 ml rice bran oil, 4 ml caprylic aid triglyceride, 6.2 g cetearyl alcohol, 8.6 g 50/50 mixture of cetearyl glucoside and cetearyl alcohol and 3.5 g cocoa butter was heated to 75° C. until dissolved, removed from heat, mixed using Cimarec 2 magnetic stir. At 45° C. 8 ml of 6 mg/ml benzamil in ethanol (0.485 mg/ml final), or 8 ml ethanol only was added together with 1.7 ml paraben and mixed. When cooled, 3 drops of wormwood oil was added, and mixed until formulated. 200 μl was topically applied per graft daily for 15 days. Effect of formulation on phenotype reversal was also validated on Rac1_{V12} transgenic mice (n=5) using 200 μl per tail per mouse per day.

Antibodies for FACS. The following antibodies were used: Brilliant Violet 785™ anti-human CD3 Antibody (317329, Biolegend), Brilliant Violet 570™ anti-human CD4 Antibody (317445, Biolegend), APC/Cy7 anti-human CD8a Antibody (300925, Biolegend), PE/Dazzle™ 594 anti-human HLA-DR Antibody (307653, Biolegend), PE Anti-human CD25 Antibody (302606, Biolegend), Alexa Fluor® 700 anti-human CD69 Antibody (310921, Biolegend), APC anti-human IL-17A Antibody (512333, BioLegend), Anti-Human/Mouse ROR gamma (t) PE (12-6988-80,ebioscience), , mouse anti-human Total Rac1 (from 16118, ThermoFisher). eBioscience

OneComp eBeads (Biolegend, 325611) were used for compensation, and Rat IgG2a K Isotype Control PE (Ebioscience, 12-4321-42) was used as isotype control.

Immunofluorescent staining. Tissue sections were embedded in OCT, snap frozen, and 7 μm sections cut on a cryostat (Leica), and sections or cells on coverslips were fixed for 10 min with cold methanol, washed with TBS, then blocked for one hour at room-temp (RT) with 10% normal goat, donkey or human serum, and incubated with primary antibody in PBS or TBS overnight. 12 hours later, washing was repeated 3 times 5 minutes, followed by incubation with secondary antibodies 1:400 together with Hoescht 1:5000 for 1 hour at RT washed and mounted with fluoromount (Southern Biotech). For mouse antibodies on mouse tissue, sections were treated with MOM IgG Blocking Kit (Vector laboratories), according to manufacturer's recommendations. Immunofluorescent slides were imaged using a Zeiss LSM 700 confocal microscope, and wholemount images using an Axioimager DIC/Fluorescence Scope (Zeiss, Oberkochen, Germany).

Antibodies for immunofluorescence staining. Phospho-STAT3 (tyr705, d3a7xp, #9145, Cell Signaling), phospho-nfKb p65 (ser536,93h1, #3033, Cell Signaling), total nf-Kb p65 (d14e12xp, #8242, Cell Signaling), CD3 (#ab5690-100, Abcam), CD49f Rat anti-Human, Unlabeled, Clone: GOH3, BD (Fisher Scientific, 555734), ki67 (ab15580, Abcam), NCK1 (Santa Cruz, sc-136232), Total Rac1 (ab155938, Abcam), NHE-1 (Santa Cruz, sc-136239 Sodium Calcium Exchanger 1/NCX1 Antibody (6H₂) (Novus, NB300-127), anti-epithelial sodium channel alpha (Abcam, ab65710), Anti-TGF alpha antibody (Abcam, ab9585) phospho-nfKb p65 (ser536,93h1, 4 #3033, Cell Signaling). The collagen polyclonal rabbit antibody used in the study has been previously described (Ortiz-Urda et al. The Journal of clinical investigation 111, 251-255 (2003).

Immunoblotting and immunoprecipitation. Cells or tissue was lysed in 1× cell lysis solution (Thermo Scientific) with 1% Halt proteinase phosphatase inhibitor (Thermo Scientific), rotated at 4° C. for 1 h and then centrifuged 15 min at 13200 rpm at 4° C. Lysates were quantified based on absorbance with a protein quantification assay (Pierce) using standard conditions. Lysates were denatured at 100° C. for 5 min with 1× NUPAGE sample loading buffer (Invitrogen), 1× NUPAGE sample reducing agent (Invitrogen), and 5% β mercaptoethanol. Subsequently, lysates were loaded on a 4-12% bis-tris gel with 1× MOPS running buffer and run for 90 min at 150 V. Gels were transferred onto nitrocellulose membranes (Amersham Protran Premium 0.45 NC) with 1× transfer buffer with 10% methanol for 2.5 h 25 V. Membranes were stained with Ponceau red, rinsed, blocked (5% milk or 5% or 3% BSA or Odyssey blocking buffer, Li—COR), washed and then incubated with primary antibody in 3% BSA or Odyssey blocking buffer overnight, washed and incubated with a HRP-tagged secondary antibody or an near IR-antibody for 1 hour at RT in 2% BSA, 5% milk, or Odyssey blocking buffer, washed and developed. Co-immunoprecipitation was performed using a co-immunoprecipitation kit (Pierce, 26149) according to manufacturer's recommendations. Briefly, 10 ug of antibody or IgG control was coupled to Aminolink Plus coupling resin, and 600 ug of protein was used per condition. For Rac1GTP pulldown, the ratio of active and total RAC1, respectively, was quantified using an Active RAC1 Pull-Down and Detection Kit (Thermo Fisher Scientific), according to the manufacturer's recommendations. Film was developed using a film developer and ECL/ECL prime, or using LI-COR technology (Odysseus TBS Blocking buffer, with IRDye® 800CW Donkey anti-Rabbit IgG (H+L) and IRDye® 680LT Donkey anti-Mouse IgG (H+L) secondary antibodies). Quantification of immunoblots was performed by densitometry in ImageJ or using Image Studio Lite (LI-COR Biosciences).

Antibodies for immunoblotting and co-immunoprecipitation. Total Rac1 (#1862341, Thermo Scientific), phospho-STAT3 (Tyr705, D3A7xp, #9145, Cell Signaling), STAT3a (D1A5xp, #8768, Cell Signaling), GAPDH (#sc-25778, Santa Cruz), pactin (#A5441, Sigma Aldrich), p44/42 MAPK (Erk1/2) (L34F12, #4696, Cell signaling), JNK1 (2C6, #3708, Cell Signaling), c-Raf (D5X6R, #12552, Cell Signaling), IRS-1(L3D12, #3194, Cell Signaling), Phospho-p44/42 MAPK, Erk1/2,Thr202/Tyr204 (P101 S, Cell signaling), Phospho-IRS-1,Ser636/639 (#2388, Cell Signaling), Phospho-SAPK/JNK, Thr183/Tyr185, (81E11, #4668, Cell Signaling), Phospho-c-Raf, Ser338, (56A6, #9427, Cell Signaling), IκBα Antibody (C-21): (Santa Cruz,sc-371). NCK1 (Santa Cruz, sc-136232, #2319 Cell Signaling).

TABLE 2
Psoriasis microarray datasets used in computational prediction
of drug targets and comparison of mRNA of benzamil targets.

| Dataset | Platform | Species | Assays |
|---|---|---|---|
| GSE6710 | HGU-133a | Homo sapiens | 10 NLS, 10 LS |
| GSE11903 | HGU-133 2.0 | Homo sapiens | 15 NLS, 14 LS |
| GSE13355 | HGU-133plus2 | Homo sapiens | 58 NLS, 58 LS, |
| | | | 64 CTLs |
| GSE14905 | HGU-133plus2 | Homo sapiens | 28 NLS, 33 LS |
| GSE30999 | HGU-133plus2 | Homo sapiens | 85 NLS, 85 LS |

TABLE 3

| | | | Adjusted | | Combined |
|---|---|---|---|---|---|
| Term | Overlap | P-value | P-value | Z-score | Score |
| IL-2 Receptor Beta Chain in T cell Activation_Homo | 49/49 | 7.31E−12 | 1.73E−09 | −1.56E+00 | 39.98 |
| sapiens_h_il2rbPathway | | | | | |
| MAPKinase Signaling Pathway_Homo | 53/56 | 1.83E−09 | 2.17E−07 | −1.46E+00 | 29.28 |
| sapiens_h_mapkPathway | | | | | |
| NFAT and Hypertrophy of the heart_Homo | 43/44 | 3.14E−09 | 2.48E−07 | −1.40E+00 | 27.49 |
| sapiens_h_nfatPathway | | | | | |
| Keratinocyte Differentiation_Homo | 50/53 | 7.50E−09 | 2.90E−07 | −1.35E+00 | 25.35 |
| sapiens_h_keratinocytePathway | | | | | |
| Rac 1 cell motility signaling pathway_Homo | 36/36 | 6.64E−09 | 2.90E−07 | −1.31E+00 | 24.63 |
| sapiens_h_rac1Pathlway | | | | | |
| HIV-1 Nef: negative effector of Fas and TNF_Homo | 50/53 | 7.50E−09 | 2.90E−07 | −1.22E+00 | 22.85 |
| sapiens_h_HivnefPathway | | | | | |
| T Cell Receptor Signaring Pathway_Homo | 51/55 | 2.71E−08 | 6.43E−07 | −1.29E+00 | 22.40 |
| sapiens_h_tcrPathway | | | | | |
| Actions of Nitric Oxide in the Heart_Homo | 41/42 | 8.57E−09 | 2.90E−07 | −1.19E+00 | 22.11 |
| sapiens_h_no1Pathway | | | | | |
| Mechanism of Gene Regulation by Peroxisome | 49/52 | 1.20E−08 | 3.55E−07 | −1.16E+00 | 21.15 |
| Proliferators via PPARa_Homo sapiens_h_pparaPathway | | | | | |
| BCR Signaling Pathway_Homo sapiens_h_bcrPathway | 31/31 | 9.10E−08 | 1.66E−06 | −1.23E+00 | 19.88 |
| ChREBP regulation by carbohydrates and cAMP_Homo | 39/40 | 2.33E−08 | 6.14E−07 | −9.83E−01 | 17.27 |
| sapiens_h_chrebpPathway | | | | | |
| Ion Channels and Their Functional Role in Vascular | 43/45 | 2.98E−08 | 6.43E−07 | −9.04E−01 | 15.67 |
| Endothelium_Homo sapiens_h_racePathway | | | | | |
| Fc Epsilon Receptor I Signaling in Mast Cells_Homo | 29/29 | 2.59E−07 | 4.09E−06 | −9.71E−01 | 14.72 |
| sapiens_h_fcer1Pathway | | | | | |
| Role of ERBB2 in Signal Transduction and Oncology_Homo | 28/28 | 4.37E−07 | 6.43E−06 | −9.79E−01 | 14.34 |
| sapiens_h_her2Pathway | | | | | |
| mCalpain and friends in Cell motility_Homo | 30/30 | 1.54E−07 | 2.60E−06 | −8.46E−01 | 13.28 |
| sapiens_h_mCalpainPathway | | | | | |
| Roles of Beta-arrestin-dependent Recruitment of Src Kinases in | 33/34 | 4.61E−07 | 6.43E−06 | −8.07E−01 | 11.77 |
| GPCR Signaling_Homo sapiens_h_bArrestin-srcPathway | | | | | |
| Rho cell motility Signaling pathway_Homo | 32/32 | 5.39E−08 | 1.06E−06 | −6.73E−01 | 11.26 |
| sapiens_h_rhoPathway | | | | | |
| Phospholipids as signalling intermediaries_Homo | 32/33 | 7.57E−07 | 8.54E−06 | −7.84E−01 | 11.04 |
| sapiens_h_edg1Pathway | | | | | |
| Bioactive Peptide Induced Signaling Pathway_Homo | 32/33 | 7.57E−07 | 8.54E−06 | −7.44E−01 | 10.48 |
| sapiens_h_biopeptidesPathway | | | | | |
| PDGF Signaling Pathway_Homo sapiens_h_pdgfPathway | 26/26 | 1.25E−06 | 1.18E−05 | −4.51E−01 | 6.13 |
| Integrin Signaling Pathway_Homo sapiens_h_integrinPathway | 32/33 | 7.57E−07 | 8.54E−06 | −3.67E−01 | 5.17 |
| Agrin in Postsynaptic Differentiation_Homo | 43/47 | 9.58E−07 | 1.03E−05 | −3.08E−01 | 4.26 |
| sapiens_h_agrPathway | | | | | |
| Signaling Pathway from G-Protein Families_Homo | 26/26 | 1.25E−06 | 1.18E−05 | −2.69E−01 | 3.66 |
| sapiens_h_gpcrPathway | | | | | |
| Control of Gene Expression by Vitamin D Receptor_Homo | 27/27 | 7.38E−07 | 8.54E−06 | −2.11E−01 | 2.98 |
| sapiens_h_vdrPathway | | | | | |

| In vivo | Route | IP | | | | |
|---|---|---|---|---|---|---|
| Vehicle | Dose (mg/kg) | Interval | Doses | Duration (days) | Effect | Assay |
| ddH20 | 10 | q2D | 20 | 20 | Full | In vivo |
| ddH20 | 2 | q2D | 20 | 40 | Full | In vivo |
| ddH20 | 1.4 | q2D | 20 | 40 | Full | In vivo |
| ddH20 | 0.7 | q2D | 20 | 40 | Partial | In vivo |

| In vivo | Route | Osmotic pump | | | | |
|---|---|---|---|---|---|---|
| Vehicle | Dose (mg/kg) | Interval | Doses | Duration (days) | Effect | Assay |
| Etoh, ddH20 | 0.6 mg/kg/day | constant | constant | 28 days | Full | In vivo |

| In vivo | Route | Topical | | | | |
|---|---|---|---|---|---|---|
| Vehicle | Dose(mg/ml) | Interval (days) | | | | |
| Vaseline | 2 | Daily | 20 | 20 | Partial | In vivo |
| ETOH | 2 | Daily | 20 | 20 | Full | In vivo |
| Labrasol based | 0.1-0.5 | Daily | 15 | 15 | Full | In vivo |
| Glycerin based | 0.485 | Daily | 15 | 15 | Full | In vivo |

| In vitro | Route | Tissue culture | | Duration(24 h | | |
|---|---|---|---|---|---|---|
| Vehicle | Dose (uM) | Interval (h) | | days) | Effect | Assay |
| ddH20 | 0.1 | 24 | 1 | 1 | Low | WB |
| ddH20 | 1 | 24 | 1 | 1 | Medium | WB |
| ddH20 | 10 | 24 | 1 | 1 | High | WB |
| ddH20 | 20 | 24 | 1 | 1 | High | WB |
| ddH20 | 50 | 24 | 1 | 1 | High | WB |
| ddH20 | 10 | 24 | 1 | 1 | High | Confocal IF cells |
| ddH20 | 0.05 | 24 | 1 | 1 | Low | MTT proliferation |
| ddH20 | 0.5 | 24 | 1 | 1 | High | MTT proliferation |
| ddH20 | 1 | 24 | 1 | 1 | High (IC50) | MTT proliferation |
| ddH20 | 2.5 | 24 | 1 | 1 | High | MTT proliferation |
| ddH20 | 5 | 24 | 1 | 1 | High | MTT proliferation |
| ddH20 | 10 | 24 | 1 | 1 | High | MTT proliferation |
| ddH20 | 10 | 24 | 1 | 1 | High | MTT proliferation |
| ddH20 | 50 | 24 | 1 | 1 | High | MTT proliferation |
| ddH20 | 50 | 24 | 1 | 1 | High | MTT proliferation |
| ddH20 | 100 | 24 | 1 | 1 | High | MTT proliferation |
| ddH20 | 10 | 24 | 7 | 7 | High | Organotypic skin |

### Example 2

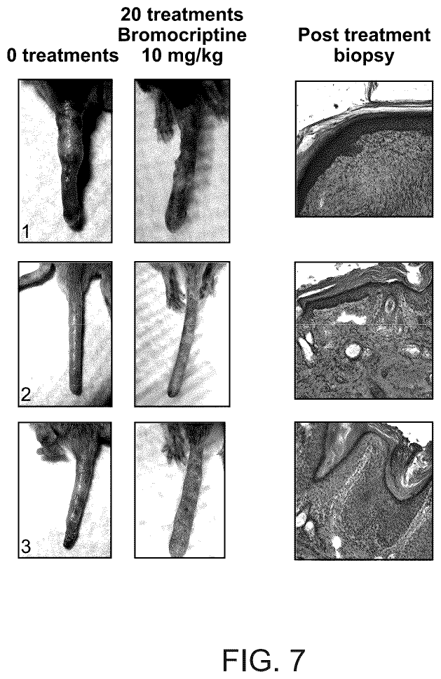

Treatment with bromocriptine. Adult Rac1 mice (n=3) with a phenotype mimicking human psoriasis were treated by intraperitoneal injection with bromocriptine (Sigma Aldrich, US) 10 mg/kg every other day for total of 20 treatments. Signs of improvement were evaluated by a competent evaluator (MCGW) and were noted to be improved after treatment, in terms of scaling, ulceration and erythema (FIG. 7A) and epidermal thickening (evaluated by H&E, FIG. 7B), compared to their baseline phenotype (Winge et al, JCI 2016).

### Example 3

### Effect of Candidate Agents on Relevant Cytokines

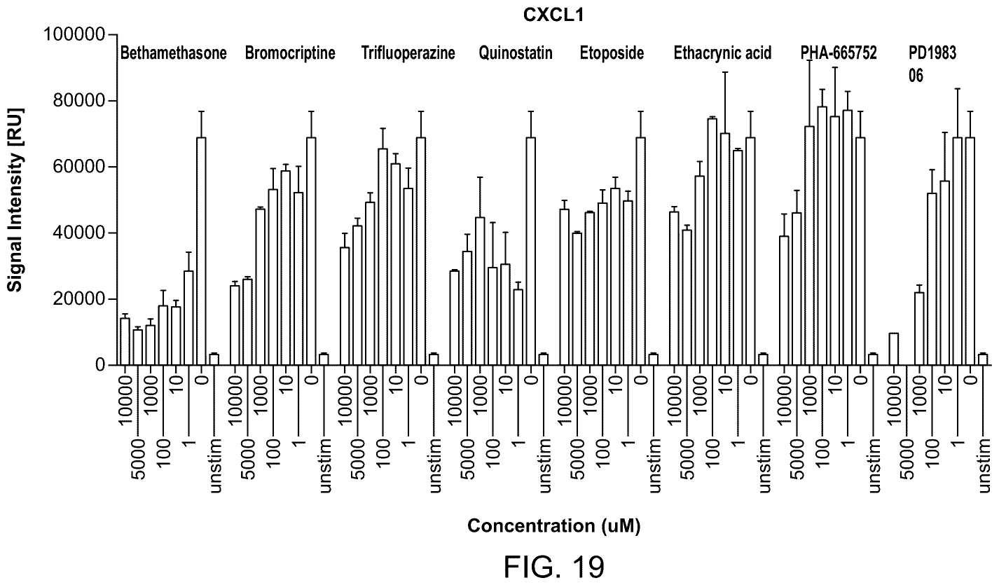

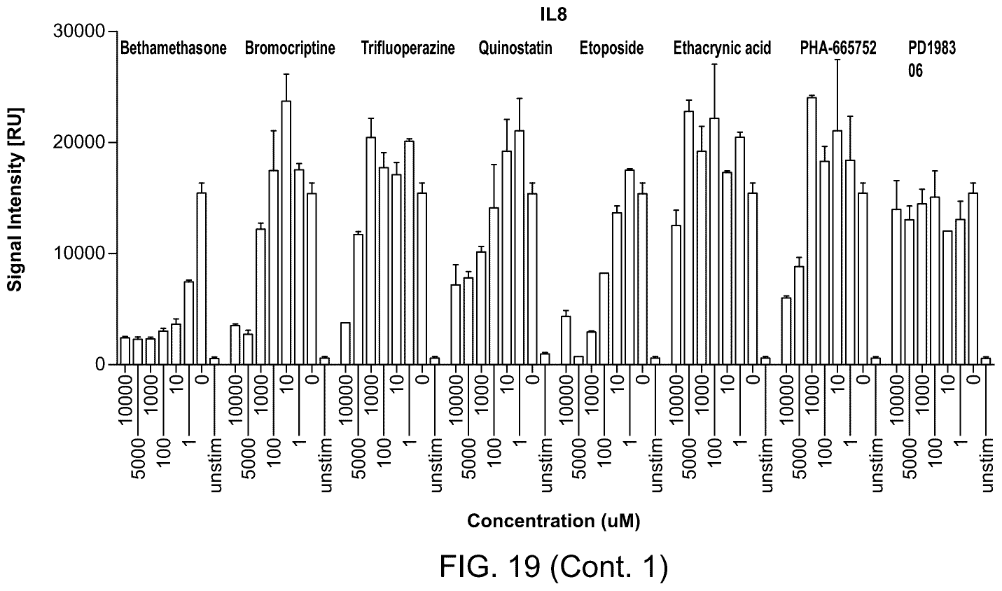

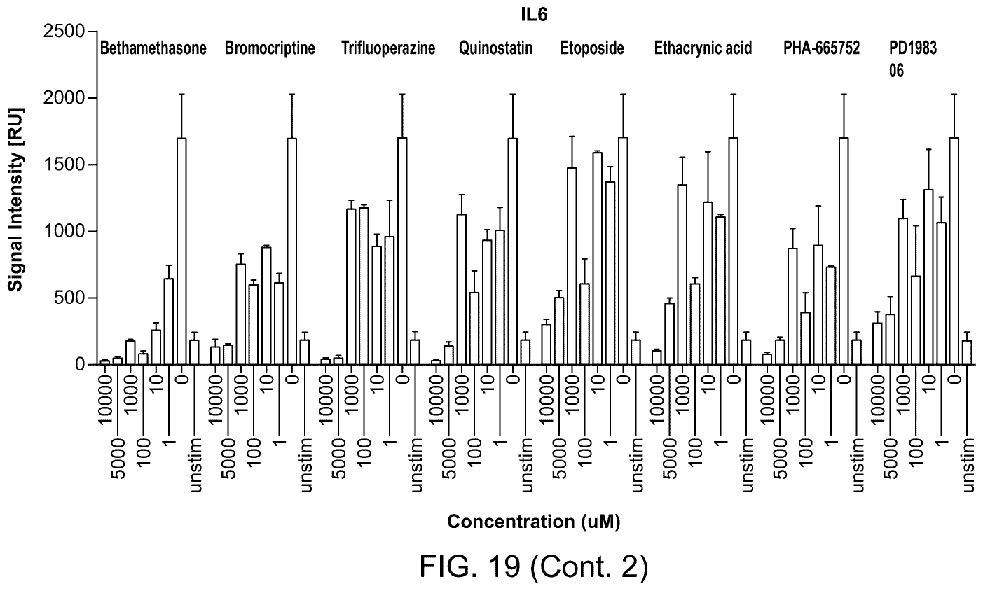

As shown in FIG. 19, there is a dose dependent decrease of CXCL1 by bromocriptine (from 1 nM) Trifluoperazine (from 1 nM), Quinostatin (from 1 nM), Etoposide (from 1 nM), Etacrynic acid (at 1, 5 and 10 uM), PHA-665752 (at 5 and 10 uM) and PD198306 (at 100 nM); and a dose-dependent decrease in IL-8 and IL-6. A decrease of IL-8 was found with Bromocriptine (at 1, 5 and 10 uM), Trifluoperazine (at 5 and 10 uM), Quinostatin (at 1, 5 and 10 uM), etoposide (at 10, 100 nM and 1, 5 and 10 uM), Etachrynic acid (at 10 uM). A decrease of IL6 by Bromocriptine (from 1 nM), Trifluoperazine, Quinostatin, Etoposide, Ethacrynic acid, PHA-665752 and PD198306 (all from 1 nM).

These data indicate that the tested compounds had psoriasis-relevant activity in the cytokine response of human keratinocytes.

### Methods:

Human Primary keratinocytes were purchased from Thermo Fisher. Keratinocytes were cultured in medium 154 supplemented with HKGS, split using TrypLE and neutralized using DMEM supplemented with 10% FBS. Cells were split to keep 20-80% confluency and not passaged above passage 5. Keratinocytes were detached using TrypLE Express, Trypsin Neutralizer solution was added and the detached cells transferred to a sterile conical tube. Cells were resuspended in 15 ml medium with HKGS supplemented (cat no S-001-K) but without hydrocortisone. The cells were pipetted up and down with a 10 ml pipette to ensure a homogeneous cell suspension.

Human primary keratinocytes detached as described above, were resuspended in PBS at 1×10⁶ cells/ml and filtered through a 35 μm cell strainer. Cells were stained with CellTrace Violet at 5 μM for 20 minutes at 37° C., 5% CO₂. After staining, DMEM supplemented with 10% FBS was added to stop staining and cells was incubated at 37° C., 5% CO₂ for 5 minutes. After incubation, cells were washed with PBS 1× and resuspended in Medium 154 supplemented with HGKS without hydrocortisone at 6×10⁵ cells/ml and 200 μl was added to wells in a 96-well flat bottom cell culture plate. Cells were incubated overnight at 37° C., 5% CO2/95% air.

Cells were incubated in duplicates at 6 concentrations stimulated with TNF-α at 10 ng/ml and IL-17 at 10 ng/ml diluted in Medium 154 supplemented with HGKS without hydrocortisone for 48 hours. Following incubation with drugs and cytokine activation for 48 hours, supernatants were transferred to a new plate and stored at −20° C. until cytokine analysis using Luminex technology.

Supernatants were analyzed for cytokines IL-6, IL-8, and CXCL1 using Luminex. All reagents, standard and samples were prepared according to manufacturer's instruction. Briefly, microparticles was added to plates and washed 2×. 50 μl of standard or sample was added to wells and samples incubated for 30 minutes at room temperature on a horizontal orbital shaker (800 rpm). Plates were washed 3× in washing buffer using a magnetic plate. Biotin-antibody was added and incubated in the dark for 30 minutes at room temperature on a horizontal orbital shaker (800 rpm). Plates were washed 3× in washing buffer using a magnetic plate. Streptavidin-PE was added to each well and incubated dark at room temperature on a horizontal orbital shaker (800 rpm) for 10 minutes. Plates were washed 3× in washing buffer using a magnetic plate. Microparticles were resuspended in 125 μl assay buffer and incubated for 30 seconds on a horizontal orbital shaker (800 rpm). Samples were analyzed on a Bio-Rad Luminex 200 analyzer. All experiments were performed at Redoxis AB, Lund, Sweden

Reagents: DMEM (VWR, L0102-500); Fetal Bovine Serum (Biowest, S181 BH); Human keratinocyte growth supplement (HKGS) kit (Thermo Fisher, S001K); Keratinocytes; Penicillin/Strepomycin (Biowhittaker, DE17-602E); PBS (Gibco, Life Technologies, 14190); Luminex 5-plex (Bio-Rad); Medium 152 (Thermo Fisher, M154500); Recombinant human TNF-α (Thermo Fisher, PHC3016); Recombinant human IL-17 (Thermo Fisher, PHC9171); mqH₂O (QPAK1, Millipore).

### REFERENCES

J. M. Crow, Psoriasis uncovered. Nature 492, S50-51 (2012).
W. H. Boehncke, M. P. Schon, Psoriasis. Lancet 386, 983-994 (2015).
J. E. Greb et al., Psoriasis. Nat Rev Dis Primers 2, 16082 (2016).
R. E. Kalb et al., Treatment of intertriginous psoriasis: from the Medical Board of the National Psoriasis Foundation. J Am Acad Dermatol 60, 120-124 (2009).
P. Di Meglio, F. Villanova, F. O. Nestle, Psoriasis. Cold Spring Harb Perspect Med 4, (2014).
C. L. Leonardi et al., Efficacy and safety of ustekinumab, a human interleukin-12/23 monoclonal antibody, in patients with psoriasis: 76-week results from a randomised, double-blind, placebo-controlled trial (PHOENIX 1). Lancet 371, 1665-1674 (2008).
K. A. Papp et al., Risankizumab versus Ustekinumab for Moderate-to-Severe Plaque Psoriasis. N Engl J Med 376, 1551-1560 (2017).
K. B. Gordon et al., Phase 3 Trials of Ixekizumab in Moderate-to-Severe Plaque Psoriasis. N Engl J Med 375, 345-356 (2016).
J. Lamb et al., The Connectivity Map: using gene-expression signatures to connect small molecules, genes, and disease. Science 313, 1929-1935 (2006).
J. T. Dudley et al., Computational repositioning of the anticonvulsant topiramate for inflammatory bowel disease. Sci Transl Med 3, 96ra76 (2011).
B. Chen et al., Reversal of cancer gene expression correlates with drug efficacy and reveals therapeutic targets. Nat Commun 8, 16022 (2017).
F. lorio, R. Tagliaferri, D. di Bernardo, Identifying network of drug mode of action by gene expression profiling. J Comput Biol 16, 241-251 (2009).
A. W. Cuthbert, J. M. Edwardson, Synthesis, properties and biological activity of tritiated N-benzylamidino-3,5-diamino-6-chloro-pyrazine carboxamide—a new ligand for epithelial sodium channels. J Pharm Pharmacol 31, 382-386 (1979).
M. Sirota et al., Discovery and preclinical validation of drug indications using compendia of public gene expression data. Sci Transl Med 3, 96ra77 (2011).
H. C. Rodgers, A. J. Knox, The effect of topical benzamil and amiloride on nasal potential difference in cystic fibrosis. Eur Respir J 14, 693-696 (1999).
M. C. Winge et al., RAC1 activation drives pathologic interactions between the epidermis and immune cells. J Clin Invest 126, 2661-2677 (2016).
M. C. Winge, M. P. Marinkovich, Epidermal activation of the small GTPase Rac1 in psoriasis pathogenesis. Small GTPases, 0 (2017).
J. M. Fuhriman et al., ITK and RLK Inhibitor PRN694 Improves Skin Disease in Two Mouse Models of Psoriasis. J Invest Dermatol 138, 864-871 (2018).
A. T. Kurdi et al., Tiam1/Rac1 complex controls Il17a transcription and autoimmunity. Nat Commun 7, 13048 (2016).
M. Koivusalo et al., Amiloride inhibits macropinocytosis by lowering submembranous pH and preventing Rac1 and Cdc42 signaling. J Cell Biol 188, 547-563 (2010).
M. J. Behne et al., NHE1 regulates the stratum corneum permeability barrier homeostasis. Microenvironment acidification assessed with fluorescence lifetime imaging. J Biol Chem 277, 47399-47406 (2002).
R. P. Charles et al., Postnatal requirement of the epithelial sodium channel for maintenance of epidermal barrier function. J Biol Chem 283, 2622-2630 (2008).
T. Dainichi, R. Matsumoto, A. Mostafa, K. Kabashima, Immune Control by TRAF6-Mediated Pathways of Epithelial Cells in the EIME (Epithelial Immune Microenvironment). Front Immunol 10, 1107 (2019).
A. Schaefer, N. R. Reinhard, P. L. Hordijk, Toward understanding RhoGTPase specificity: structure, function and local activation. Small GTPases 5, 6 (2014).
K. J. Roux, D. I. Kim, B. Burke, BiolD: a screen for protein-protein interactions. Curr Protoc Protein Sci 74, Unit 19 23 (2013).
D. Szklarczyk et al., STRING v10: protein-protein interaction networks, integrated over the tree of life. Nucleic Acids Res 43, D447-452 (2015).
A. Gutierrez-Uzquiza et al., Coordinated activation of the Rac-GAP beta2-chimaerin by an atypical proline-rich domain and diacylglycerol. Nat Commun 4, 1849 (2013).
Y. Baumer et al., Chronic skin inflammation accelerates macrophage cholesterol crystal formation and atherosclerosis. JCI Insight 3, (2018).
M. Shekarabi et al., Deleted in colorectal cancer binding netrin-1 mediates cell substrate adhesion and recruits Cdc42, Rac1, Paki, and N-WASP into an intracellular signaling complex that promotes growth cone expansion. J Neurosci 25, 3132-3141 (2005).
N. Xu et al., MicroRNA-31 is overexpressed in psoriasis and modulates inflammatory cytokine and chemokine production in keratinocytes via targeting serine/threonine kinase 40. J Immunol 190, 678-688 (2013).
J. C. Martin et al., Limited Presence of IL-22 Binding Protein, a Natural IL-22 Inhibitor, Strengthens Psoriatic Skin Inflammation. J Immunol 198, 3671-3678 (2017).
S. H. Ahn et al., IL-17A Contributes to the Pathogenesis of Endometriosis by Triggering Proinflammatory Cytokines and Angiogenic Growth Factors. J Immunol 195, 2591-2600 (2015).
S. M. Sa et al., The effects of IL-20 subfamily cytokines on reconstituted human epidermis suggest potential roles in cutaneous innate defense and pathogenic adaptive immunity in psoriasis. J Immunol 178, 2229-2240 (2007).
Y. Zheng et al., Interleukin-22, a T(H)17 cytokine, mediates IL-23-induced dermal inflammation and acanthosis. Nature 445, 648-651 (2007). 69
S. Lorscheid et al., Keratinocyte-derived IkappaBzeta drives psoriasis and associated systemic inflammation. JCI Insight 4, (2019).
H. L. Ha, H. Wang, E. Claudio, W. Tang, U. Siebenlist, IL-20-Receptor Signaling Delimits IL-17 Production in Psoriatic Inflammation. J Invest Dermatol 140, 143-151 e143 (2020).
M. A. Magalhaes et al., Cortactin phosphorylation regulates cell invasion through a pH-dependent pathway. J Cell Biol 195, 903-920 (2011).
T. Mauro et al., The ENaC channel is required for normal epidermal differentiation. J Invest Dermatol 118, 589-594 (2002).
J. R. Abreu et al., A Rac1 inhibitory peptide suppresses antibody production and paw swelling in the murine collagen-induced arthritis model of rheumatoid arthritis. Arthritis Res Ther 12, R2 (2010).
A. J. Hirsh et al., Evaluation of second generation amiloride analogs as therapy for cystic fibrosis lung disease. J Pharmacol Exp Ther 311, 929-938 (2004).
A. Kapus, G. L. Lukacs, E. J. Cragoe, Jr., E. Ligeti, A. Fonyo, Characterization of the mitochondrial Na+—H+ exchange. The effect of amiloride analogues. Biochim Biophys Acta 944, 383-390 (1988).
J. R. Casey, S. Grinstein, J. Orlowski, Sensors and regulators of intracellular pH. Nat Rev Mol Cell Biol 11, 50-61 (2010).
F. Li et al., GeneExpressionSignature: an R package for discovering functional connections using gene expression signatures. OMICS 17, 116-118 (2013).
A. Subramanian, H. Kuehn, J. Gould, P. Tamayo, J. P. Mesirov, GSEA-P: a desktop application for Gene Set Enrichment Analysis. Bioinformatics 23, 3251-3253 (2007).
D. Merico, R. Isserlin, G. D. Bader, Visualizing gene-set enrichment results using the Cytoscape plug-in enrichment map. Methods Mol Biol 781, 257-277 (2011).
M. J. Keiser, J. Hert, Off-target networks derived from ligand set similarity. Methods Mol Biol 575, 71 195-205 (2009).
M. J. Keiser et al., Relating protein pharmacology by ligand chemistry. Nat Biotechnol 25, 197-206 (2007).
N. E. Sanjana, O. Shalem, F. Zhang, Improved vectors and genome-wide libraries for CRISPR screening. Nat Methods 11, 783-784 (2014).
O. Shalem et al., Genome-scale CRISPR-Cas9 knockout screening in human cells. Science 343, 84-87 (2014).
V. Lopez-Pajares et al., A LncRNA-MAF:MAFB transcription factor network regulates epidermal differentiation. Dev Cell 32, 693-706 (2015).

1. A method of treating psoriasis in a subject, the method comprising:
administering to the subject an effective dose of a compound of Table 1 for duration and periodicity sufficient to reduce the symptoms of psoriasis.

2. The method of claim 1, wherein the anti-psoriasis agent is selected from quinostatin 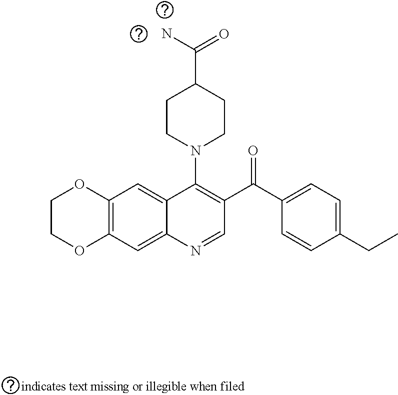 [SMILES: CCc1ccc(C(=O)c2cnc3cc4c(cc3c2N2CCC(C(N)=O)CC2)OCCO4)cc1]
or a derivative thereof; bromocriptine 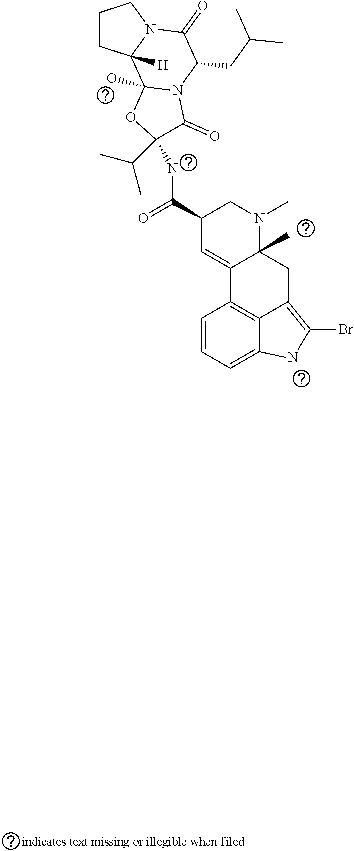
or a derivative thereof; trifluoperazine 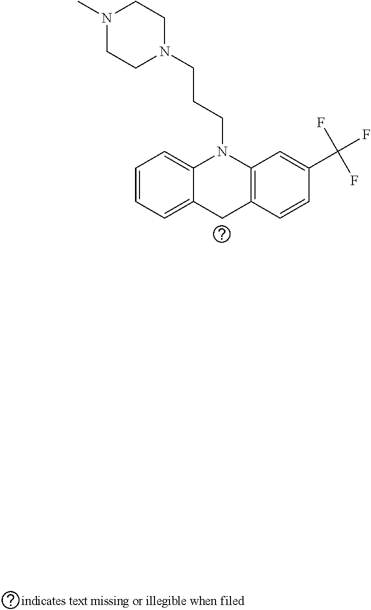 [SMILES: CN1CCN(CCCN2c3ccccc3Cc3ccc(C(F)(F)F)cc32)CC1]
or a derivative thereof; sirolimus 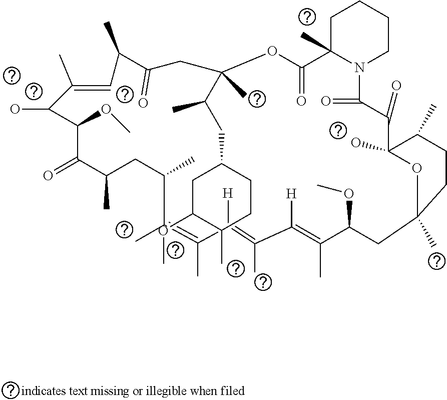
or a derivative thereof; 0297417-0002B 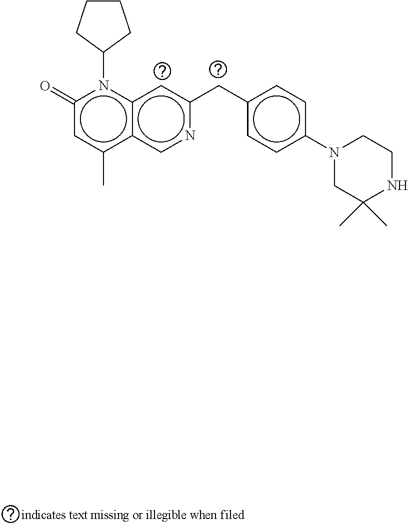
or a derivative thereof; 0198306-0000, PD-198306 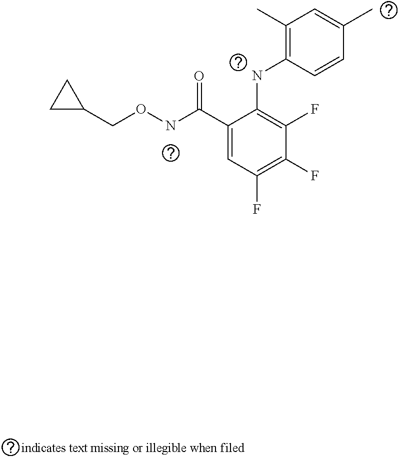 [SMILES: Cc1ccc(Nc2c(C(=O)NOCC3CC3)cc(F)c(F)c2F)c(C)c1]
or a derivative thereof; PHA-00665752 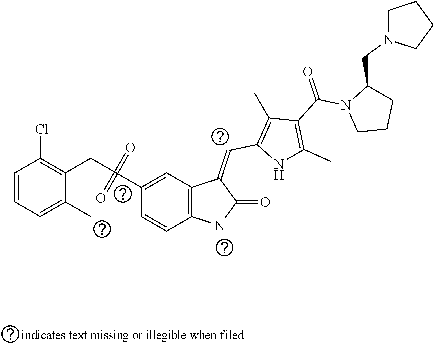
or a derivative thereof; PNU-0230031 [267429-39-0] 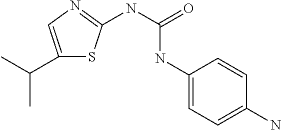 [SMILES: CC(C)c1cnc(NC(=O)Nc2ccc(N)cc2)s1]
or a derivative thereof; AG-012559 [369370-06-9] 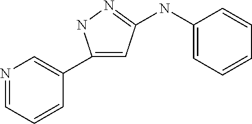 [SMILES: c1ccc(Nc2cc(-c3cccnc3)[nH]n2)cc1]
or a derivative thereof; etoposide 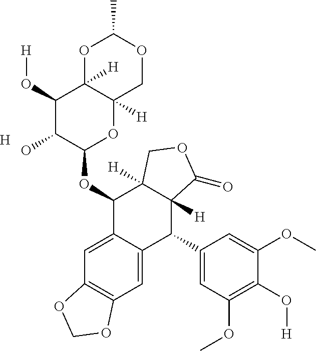
or a derivative thereof; or ethacrynic acid 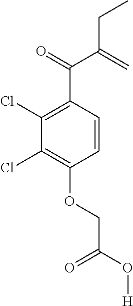 [SMILES: C=C(CC)C(=O)c1ccc(OCC(=O)O)c(Cl)c1Cl]
or a derivative thereof.

3-12. (canceled)

13. The method of claim 2, wherein the administration is systemic.

14. The method of claim 2, wherein the administration is topical.

15. The method of claim 2, wherein dose is administered to the subject upon a flare of psoriasis.

16. The method of claim 2, wherein the psoriasis is chronic psoriasis.

17. The method of claim 2, wherein the psoriasis is plaque psoriasis.

18. A composition in a unit dose formulation of an anti-psoriasis agent of Table 1, suitable for use in the methods of claim 2.

19. The composition of claim 18, wherein the anti-psoriasis agent is quinostatin 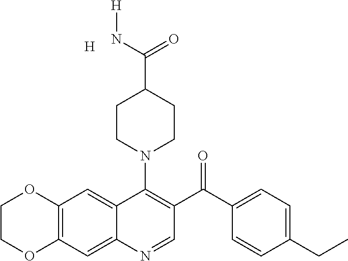
or a derivative thereof.

20. The composition of claim 18, wherein the anti-psoriasis agent is bromocriptine 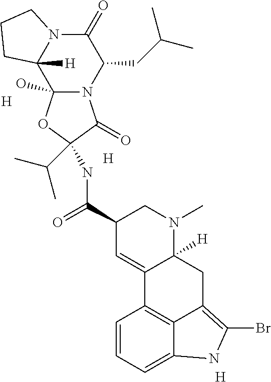
or a derivative thereof.

21. The composition of claim 18, wherein the anti-psoriasis agent is trifluoperazine 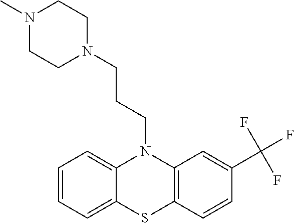 [SMILES: CN1CCN(CCCN2c3ccccc3Sc3ccc(C(F)(F)F)cc32)CC1]
or a derivative thereof.

22. The composition of claim 18, wherein the anti-psoriasis agent is sirolimus 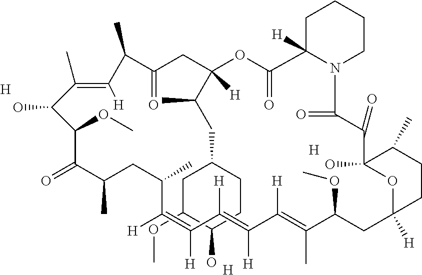
or a derivative thereof.

23. The composition of claim 18, wherein the anti-psoriasis agent is 0297417-0002B 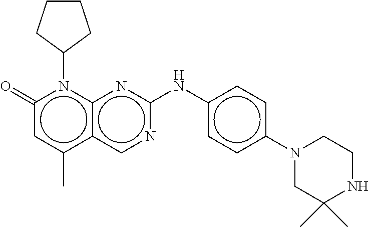
or a derivative thereof.

24. The composition of claim 18, wherein the anti-psoriasis agent is 0198306-0000, PD-198306 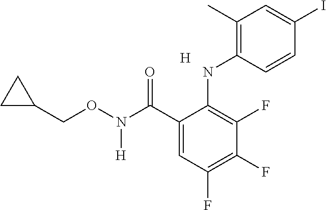
or a derivative thereof.

25. The composition of claim 18, wherein the anti-psoriasis agent is PHA-00665752 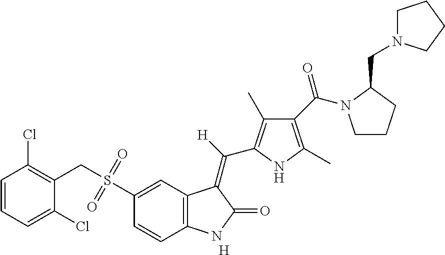
or a derivative thereof.

26. The composition of claim 18, wherein the anti-psoriasis agent is PNU-0230031 [267429-39-0] 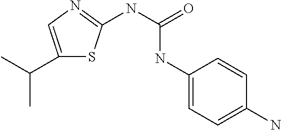 [SMILES: CC(C)c1cnc(NC(=O)Nc2ccc(N)cc2)s1]
or a derivative thereof.

27. The composition of claim 18, wherein the anti-psoriasis agent is AG-012559 [369370-06-9] 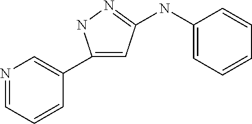 [SMILES: c1ccc(Nc2cc(-c3cccnc3)[nH]n2)cc1]
or a derivative thereof.

28. The composition of claim 18, wherein the anti-psoriasis agent is etoposide 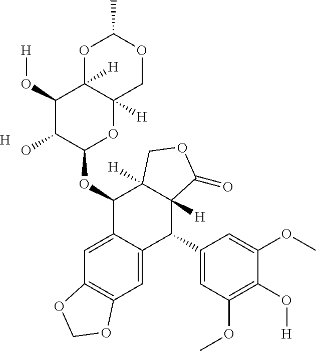
or a derivative thereof.

29. The composition of claim 18, wherein the anti-psoriasis agent is ethacrynic acid 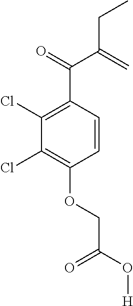 [SMILES: C=C(CC)C(=O)c1ccc(OCC(=O)O)c(Cl)c1Cl]
or a derivative thereof.

30. The composition of claim 18, wherein the anti-psoriasis agent is the sole active agent.

31. The composition of claim 18, further comprising a second therapeutic agent for treatment of psoriasis.

32-33. (canceled)
